SEQUENCE LISTING (1006 sequences)

SEQ ID NO: 1-500 - skipped (500 entries)

SEQ ID NO: 501 - protein, 5 aa, synthetic construct
  REGION 1..5: note = Synthetic peptide
TYWMH

SEQ ID NO: 502 - protein, 17 aa, synthetic construct
  REGION 1..17: note = Synthetic peptide
NIYPGTGGSNFDEKFKN

SEQ ID NO: 503 - protein, 8 aa, synthetic construct
  REGION 1..8: note = Synthetic peptide
WTTGTGAY

SEQ ID NO: 504 - protein, 7 aa, synthetic construct
  REGION 1..7: note = Synthetic peptide
GYTFTTY

SEQ ID NO: 505 - protein, 6 aa, synthetic construct
  REGION 1..6: note = Synthetic peptide
YPGTGG

SEQ ID NO: 506 - protein, 117 aa, synthetic construct
  REGION 1..117: note = Synthetic peptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, synthetic construct
  misc_feature 1..351: note = Synthetic polynucleotide
  source 1..351: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, synthetic construct
  REGION 1..443: note = Synthetic peptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, synthetic construct
  misc_feature 1..1329: note = Synthetic polynucleotide
  source 1..1329: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, synthetic construct
  REGION 1..17: note = Synthetic peptide
KSSQSLLDSGNQKNFLT

SEQ ID NO: 511 - protein, 7 aa, synthetic construct
  REGION 1..7: note = Synthetic peptide
WASTRES

SEQ ID NO: 512 - protein, 9 aa, synthetic construct
  REGION 1..9: note = Synthetic peptide
QNDYSYPYT

SEQ ID NO: 513 - protein, 13 aa, synthetic construct
  REGION 1..13: note = Synthetic peptide
SQSLLDSGNQKNF

SEQ ID NO: 514 - skipped

SEQ ID NO: 515 - protein, 6 aa, synthetic construct
  REGION 1..6: note = Synthetic peptide
DYSYPY

SEQ ID NO: 516 - protein, 113 aa, synthetic construct
  REGION 1..113: note = Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, synthetic construct
  misc_feature 1..339: note = Synthetic polynucleotide
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, synthetic construct
  REGION 1..220: note = Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, synthetic construct
  misc_feature 1..660: note = Synthetic polynucleotide
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, synthetic construct
  REGION 1..113: note = Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, synthetic construct
  misc_feature 1..339: note = Synthetic polynucleotide
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, synthetic construct
  REGION 1..220: note = Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, synthetic construct
  misc_feature 1..660: note = Synthetic polynucleotide
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, synthetic construct
  misc_feature 1..15: note = Synthetic oligonucleotide
  source 1..15: mol_type = other DNA
acctactggatgcac

SEQ ID NO: 525 - DNA, 51 bp, synthetic construct
  misc_feature 1..51: note = Synthetic oligonucleotide
  source 1..51: mol_type = other DNA
aatatctaccccggcaccggcggctctaacttcgacgagaagtttaagaat

SEQ ID NO: 526 - DNA, 24 bp, synthetic construct
  misc_feature 1..24: note = Synthetic oligonucleotide
  source 1..24: mol_type = other DNA
tggactaccggcacaggcgcctac

SEQ ID NO: 527 - DNA, 21 bp, synthetic construct
  misc_feature 1..21: note = Synthetic oligonucleotide
  source 1..21: mol_type = other DNA
ggctacaccttcactacctac

SEQ ID NO: 528 - DNA, 18 bp, synthetic construct
  misc_feature 1..18: note = Synthetic oligonucleotide
  source 1..18: mol_type = other DNA
taccccggcaccggcggc

SEQ ID NO: 529 - DNA, 51 bp, synthetic construct
  misc_feature 1..51: note = Synthetic oligonucleotide
  source 1..51: mol_type = other DNA
aaatctagtcagtcactgctggatagcggtaatcagaagaacttcctgacc

SEQ ID NO: 530 - DNA, 21 bp, synthetic construct
  misc_feature 1..21: note = Synthetic oligonucleotide
  source 1..21: mol_type = other DNA
tgggcctctactagagaatca

SEQ ID NO: 531 - DNA, 27 bp, synthetic construct
  misc_feature 1..27: note = Synthetic oligonucleotide
  source 1..27: mol_type = other DNA
cagaacgactatagctacccctacacc

SEQ ID NO: 532 - DNA, 39 bp, synthetic construct
  misc_feature 1..39: note = Synthetic oligonucleotide
  source 1..39: mol_type = other DNA
agtcagtcactgctggatagcggtaatcagaagaacttc

SEQ ID NO: 533 - skipped

SEQ ID NO: 534 - DNA, 18 bp, synthetic construct
  misc_feature 1..18: note = Synthetic oligonucleotide
  source 1..18: mol_type = other DNA
gactatagctacccctac

SEQ ID NO: 535 - protein, 440 aa, synthetic construct
  REGION 1..440: note = Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, synthetic construct
  REGION 1..214: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, synthetic construct
  REGION 1..447: note = Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, synthetic construct
  REGION 1..218: note = Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, synthetic construct
  REGION 1..447: note = Synthetic polypeptide
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, synthetic construct
  REGION 1..213: note = Synthetic polypeptide
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, synthetic construct
  REGION 1..10: note = Synthetic peptide
GYTFTTYWMH

SEQ ID NO: 542-600 - skipped (59 entries)

SEQ ID NO: 601 - protein, 5 aa, synthetic construct
  REGION 1..5: note = Synthetic peptide
SYWMY

SEQ ID NO: 602 - protein, 17 aa, synthetic construct
  REGION 1..17: note = Synthetic peptide
RIDPNSGSTKYNEKFKN

SEQ ID NO: 603 - protein, 11 aa, synthetic construct
  REGION 1..11: note = Synthetic peptide
DYRKGLYAMDY

SEQ ID NO: 604 - protein, 7 aa, synthetic construct
  REGION 1..7: note = Synthetic peptide
GYTFTSY

SEQ ID NO: 605 - protein, 6 aa, synthetic construct
  REGION 1..6: note = Synthetic peptide
DPNSGS

SEQ ID NO: 606 - protein, 120 aa, synthetic construct
  REGION 1..120: note = Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, synthetic construct
  misc_feature 1..360: note = Synthetic polynucleotide
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, synthetic construct
  REGION 1..446: note = Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, synthetic construct
  REGION 1..11: note = Synthetic peptide
KASQDVGTAVA

SEQ ID NO: 610 - protein, 7 aa, synthetic construct
  REGION 1..7: note = Synthetic peptide
WASTRHT

SEQ ID NO: 611 - protein, 9 aa, synthetic construct
  REGION 1..9: note = Synthetic peptide
QQYNSYPLT

SEQ ID NO: 612 - protein, 7 aa, synthetic construct
  REGION 1..7: note = Synthetic peptide
SQDVGTA

SEQ ID NO: 613 - skipped

SEQ ID NO: 614 - protein, 6 aa, synthetic construct
  REGION 1..6: note = Synthetic peptide
YNSYPL

SEQ ID NO: 615 - DNA, 1338 bp, synthetic construct
  misc_feature 1..1338: note = Synthetic polynucleotide
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, synthetic construct
  REGION 1..107: note = Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, synthetic construct
  misc_feature 1..321: note = Synthetic polynucleotide
  source 1..321: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, synthetic construct
  REGION 1..214: note = Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, synthetic construct
  misc_feature 1..642: note = Synthetic polynucleotide
  source 1..642: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, synthetic construct
  REGION 1..120: note = Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, synthetic construct
  misc_feature 1..360: note = Synthetic polynucleotide
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, synthetic construct
  REGION 1..446: note = Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, synthetic construct
  misc_feature 1..1338: note = Synthetic polynucleotide
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, synthetic construct
  REGION 1..107: note = Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, synthetic construct
  misc_feature 1..321: note = Synthetic polynucleotide
  source 1..321: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, synthetic construct
  REGION 1..214: note = Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, synthetic construct
  misc_feature 1..642: note = Synthetic polynucleotide
  source 1..642: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, synthetic construct
  misc_feature 1..15: note = Synthetic oligonucleotide
  source 1..15: mol_type = other DNA
agctactggatgtac

SEQ ID NO: 629 - DNA, 51 bp, synthetic construct
  misc_feature 1..51: note = Synthetic oligonucleotide
  source 1..51: mol_type = other DNA
agaatcgaccctaatagcggctctactaagtataacgagaagtttaagaat

SEQ ID NO: 630 - DNA, 33 bp, synthetic construct
  misc_feature 1..33: note = Synthetic oligonucleotide
  source 1..33: mol_type = other DNA
gactatagaaagggcctgtacgctatggactac

SEQ ID NO: 631 - DNA, 21 bp, synthetic construct
  misc_feature 1..21: note = Synthetic oligonucleotide
  source 1..21: mol_type = other DNA
ggctacaccttcactagctac

SEQ ID NO: 632 - DNA, 18 bp, synthetic construct
  misc_feature 1..18: note = Synthetic oligonucleotide
  source 1..18: mol_type = other DNA
gaccctaatagcggctct

SEQ ID NO: 633 - DNA, 33 bp, synthetic construct
  misc_feature 1..33: note = Synthetic oligonucleotide
  source 1..33: mol_type = other DNA
aaagcctctcaggacgtgggcaccgccgtggcc

SEQ ID NO: 634 - DNA, 21 bp, synthetic construct
  misc_feature 1..21: note = Synthetic oligonucleotide
  source 1..21: mol_type = other DNA
tgggcctctactagacacacc

SEQ ID NO: 635 - DNA, 27 bp, synthetic construct
  misc_feature 1..27: note = Synthetic oligonucleotide
  source 1..27: mol_type = other DNA
cagcagtataatagctaccccctgacc

SEQ ID NO: 636 - DNA, 21 bp, synthetic construct
  misc_feature 1..21: note = Synthetic oligonucleotide
  source 1..21: mol_type = other DNA
tctcaggacgtgggcaccgcc

SEQ ID NO: 637 - skipped

SEQ ID NO: 638 - DNA, 18 bp, synthetic construct
  misc_feature 1..18: note = Synthetic oligonucleotide
  source 1..18: mol_type = other DNA
tataatagctaccccctg

SEQ ID NO: 639 - protein, 448 aa, synthetic construct
  REGION 1..448: note = Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, synthetic construct
  REGION 1..214: note = Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, synthetic construct
  REGION 1..450: note = Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, synthetic construct
  REGION 1..216: note = Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, synthetic construct
  REGION 1..451: note = Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, synthetic construct
  REGION 1..215: note = Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, synthetic construct
  REGION 1..123: note = Synthetic polypeptide
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, synthetic construct
  REGION 1..106: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, synthetic construct
  REGION 1..10: note = Synthetic peptide
GYTFTSYWMY

SEQ ID NO: 648-700 - skipped (53 entries)

SEQ ID NO: 701 - protein, 5 aa, synthetic construct
  REGION 1..5: note = Synthetic peptide
NYGMN

SEQ ID NO: 702 - protein, 17 aa, synthetic construct
  REGION 1..17: note = Synthetic peptide
WINTDTGEPTYADDFKG

SEQ ID NO: 703 - protein, 16 aa, synthetic construct
  REGION 1..16: note = Synthetic peptide
NPPYYYGTNNAEAMDY

SEQ ID NO: 704 - protein, 7 aa, synthetic construct
  REGION 1..7: note = Synthetic peptide
GFTLTNY

SEQ ID NO: 705 - protein, 6 aa, synthetic construct
  REGION 1..6: note = Synthetic peptide
NTDTGE

SEQ ID NO: 706 - protein, 125 aa, synthetic construct
  REGION 1..125: note = Synthetic polypeptide
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, synthetic construct
  misc_feature 1..375: note = Synthetic polynucleotide
  source 1..375: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, synthetic construct
  misc_feature 1..375: note = Synthetic polynucleotide
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, synthetic construct
  REGION 1..451: note = Synthetic polypeptide
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, synthetic construct
  REGION 1..11: note = Synthetic peptide
SSSQDISNYLN

SEQ ID NO: 711 - protein, 7 aa, synthetic construct
  REGION 1..7: note = Synthetic peptide
YTSTLHL

SEQ ID NO: 712 - protein, 9 aa, synthetic construct
  REGION 1..9: note = Synthetic peptide
QQYYNLPWT

SEQ ID NO: 713 - protein, 7 aa, synthetic construct
  REGION 1..7: note = Synthetic peptide
SQDISNY

SEQ ID NO: 714 - skipped

SEQ ID NO: 715 - protein, 6 aa, synthetic construct
  REGION 1..6: note = Synthetic peptide
YYNLPW

SEQ ID NO: 716 - DNA, 1353 bp, synthetic construct
  misc_feature 1..1353: note = Synthetic polynucleotide
  source 1..1353: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, synthetic construct
  misc_feature 1..1353: note = Synthetic polynucleotide
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, synthetic construct
  REGION 1..107: note = Synthetic polypeptide
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, synthetic construct
  misc_feature 1..321: note = Synthetic polynucleotide
  source 1..321: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, synthetic construct
  misc_feature 1..321: note = Synthetic polynucleotide
  source 1..321: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, synthetic construct
  REGION 1..214: note = Synthetic polypeptide
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, synthetic construct
  misc_feature 1..642: note = Synthetic polynucleotide
  source 1..642: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, synthetic construct
  misc_feature 1..642: note = Synthetic polynucleotide
  source 1..642: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, synthetic construct
  REGION 1..125: note = Synthetic polypeptide
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, synthetic construct
  misc_feature 1..375: note = Synthetic polynucleotide
  source 1..375: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, synthetic construct
  misc_feature 1..375: note = Synthetic polypeptide
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, synthetic construct
  REGION 1..451: note = Synthetic polynucleotide
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, synthetic construct
  misc_feature 1..1353: note = Synthetic polynucleotide
  source 1..1353: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, synthetic construct
  misc_feature 1..1353: note = Synthetic polynucleotide
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, synthetic construct
  REGION 1..107: note = Synthetic polypeptide
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, synthetic construct
  misc_feature 1..321: note = Synthetic polynucleotide
  source 1..321: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, synthetic construct
  misc_feature 1..321: note = Synthetic polynucleotide
  source 1..321: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, synthetic construct
  REGION 1..214: note = Synthetic polypeptide
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, synthetic construct
  misc_feature 1..642: note = Synthetic polynucleotide
  source 1..642: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, synthetic construct
  misc_feature 1..642: note = Synthetic polynucleotide
  source 1..642: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, synthetic construct
  misc_feature 1..15: note = Synthetic oligonucleotide
  source 1..15: mol_type = other DNA
aattacgggatgaac

SEQ ID NO: 737 - DNA, 15 bp, synthetic construct
  misc_feature 1..15: note = Synthetic oligonucleotide
  source 1..15: mol_type = other DNA
aactacggcatgaac

SEQ ID NO: 738 - DNA, 51 bp, synthetic construct
  misc_feature 1..51: note = Synthetic oligonucleotide
  source 1..51: mol_type = other DNA
tggattaacaccgacaccggggagcctacctacgcggacgatttcaaggga

SEQ ID NO: 739 - DNA, 51 bp, synthetic construct
  misc_feature 1..51: note = Synthetic oligonucleotide
  source 1..51: mol_type = other DNA
tggatcaacaccgacaccggcgagcctacctacgccgacgacttcaagggc

SEQ ID NO: 740 - DNA, 48 bp, synthetic construct
  misc_feature 1..48: note = Synthetic oligonucleotide
  source 1..48: mol_type = other DNA
aacccgccctactactacggaaccaacaacgccgaagccatggactac

SEQ ID NO: 741 - DNA, 48 bp, synthetic construct
  misc_feature 1..48: note = Synthetic oligonucleotide
  source 1..48: mol_type = other DNA
aaccccccttactactacggcaccaacaacgccgaggccatggactat

SEQ ID NO: 742 - DNA, 21 bp, synthetic construct
  misc_feature 1..21: note = Synthetic oligonucleotide
  source 1..21: mol_type = other DNA
ggattcaccctcaccaattac

SEQ ID NO: 743 - DNA, 21 bp, synthetic construct
  misc_feature 1..21: note = Synthetic oligonucleotide
  source 1..21: mol_type = other DNA
ggcttcaccctgaccaactac

SEQ ID NO: 744 - DNA, 18 bp, synthetic construct
  misc_feature 1..18: note = Synthetic oligonucleotide
  source 1..18: mol_type = other DNA
aacaccgacaccggggag

SEQ ID NO: 745 - DNA, 18 bp, synthetic construct
  misc_feature 1..18: note = Synthetic oligonucleotide
  source 1..18: mol_type = other DNA
aacaccgacaccggcgag

SEQ ID NO: 746 - DNA, 33 bp, synthetic construct
  misc_feature 1..33: note = Synthetic oligonucleotide
  source 1..33: mol_type = other DNA
agctctagtcaggatatctctaactacctgaac

SEQ ID NO: 747 - DNA, 33 bp, synthetic construct
  misc_feature 1..33: note = Synthetic oligonucleotide
  source 1..33: mol_type = other DNA
tcctccagccaggacatctccaactacctgaac

SEQ ID NO: 748 - DNA, 21 bp, synthetic construct
  misc_feature 1..21: note = Synthetic oligonucleotide
  source 1..21: mol_type = other DNA
tacactagcaccctgcacctg

SEQ ID NO: 749 - DNA, 21 bp, synthetic construct
  misc_feature 1..21: note = Synthetic oligonucleotide
  source 1..21: mol_type = other DNA
tacacctccaccctgcacctg

SEQ ID NO: 750 - DNA, 27 bp, synthetic construct
  misc_feature 1..27: note = Synthetic oligonucleotide
  source 1..27: mol_type = other DNA
cagcagtactataacctgccctggacc

SEQ ID NO: 751 - DNA, 27 bp, synthetic construct
  misc_feature 1..27: note = Synthetic oligonucleotide
  source 1..27: mol_type = other DNA
cagcagtactacaacctgccctggacc

SEQ ID NO: 752 - DNA, 21 bp, synthetic construct
  misc_feature 1..21: note = Synthetic oligonucleotide
  source 1..21: mol_type = other DNA
agtcaggatatctctaactac

SEQ ID NO: 753 - DNA, 21 bp, synthetic construct
  misc_feature 1..21: note = Synthetic oligonucleotide
  source 1..21: mol_type = other DNA
agccaggacatctccaactac

SEQ ID NO: 754-755 - skipped (2 entries)

SEQ ID NO: 756 - DNA, 18 bp, synthetic construct
  misc_feature 1..18: note = Synthetic oligonucleotide
  source 1..18: mol_type = other DNA
tactataacctgccctgg

SEQ ID NO: 757 - DNA, 18 bp, synthetic construct
  misc_feature 1..18: note = Synthetic oligonucleotide
  source 1..18: mol_type = other DNA
tactacaacctgccctgg

SEQ ID NO: 758 - DNA, 15 bp, synthetic construct
  misc_feature 1..15: note = Synthetic oligonucleotide
  source 1..15: mol_type = other DNA
aactacgggatgaac

SEQ ID NO: 759 - DNA, 51 bp, synthetic construct
  misc_feature 1..51: note = Synthetic oligonucleotide
  source 1..51: mol_type = other DNA
tggattaacaccgacaccggcgagcctacctacgccgacgactttaagggc

SEQ ID NO: 760 - DNA, 48 bp, synthetic construct
  misc_feature 1..48: note = Synthetic oligonucleotide
  source 1..48: mol_type = other DNA
aaccccccctactactacggcactaacaacgccgaggctatggactac

SEQ ID NO: 761 - DNA, 21 bp, synthetic construct
  misc_feature 1..21: note = Synthetic oligonucleotide
  source 1..21: mol_type = other DNA
ggcttcaccctgactaactac

SEQ ID NO: 762 - protein, 447 aa, synthetic construct
  REGION 1..447: note = Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, synthetic construct
  REGION 1..214: note = Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, synthetic construct
  REGION 1..446: note = Synthetic polypeptide
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, synthetic construct
  REGION 1..220: note = Synthetic polypeptide
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 10 aa, synthetic construct
  REGION 1..10: note = Synthetic peptide
GFTLTNYGMN

SEQ ID NO: 767-800 - skipped (34 entries)

SEQ ID NO: 801 - protein, 5 aa, synthetic construct
  REGION 1..5: note = Synthetic peptide
SYNMH

SEQ ID NO: 802 - protein, 17 aa, synthetic construct
  REGION 1..17: note = Synthetic peptide
DIYPGNGDTSYNQKFKG

SEQ ID NO: 803 - protein, 9 aa, synthetic construct
  REGION 1..9: note = Synthetic peptide
VGGAFPMDY

SEQ ID NO: 804 - protein, 7 aa, synthetic construct
  REGION 1..7: note = Synthetic peptide
GYTFTSY

SEQ ID NO: 805 - protein, 6 aa, synthetic construct
  REGION 1..6: note = Synthetic peptide
YPGNGD

SEQ ID NO: 806 - protein, 118 aa, synthetic construct
  REGION 1..118: note = Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, synthetic construct
  misc_feature 1..354: note = Synthetic polynucleotide
  source 1..354: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, synthetic construct
  REGION 1..444: note = Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, synthetic construct
  misc_feature 1..1332: note = Synthetic polynucleotide
  source 1..1332: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, synthetic construct
  REGION 1..15: note = Synthetic peptide
RASESVEYYGTSLMQ

SEQ ID NO: 811 - protein, 7 aa, synthetic construct
  REGION 1..7: note = Synthetic peptide
AASNVES

SEQ ID NO: 812 - protein, 9 aa, synthetic construct
  REGION 1..9: note = Synthetic peptide
QQSRKDPST

SEQ ID NO: 813 - protein, 11 aa, synthetic construct
  REGION 1..11: note = Synthetic peptide
SESVEYYGTSL

SEQ ID NO: 814 - skipped

SEQ ID NO: 815 - protein, 6 aa, synthetic construct
  REGION 1..6: note = Synthetic peptide
SRKDPS

SEQ ID NO: 816 - protein, 111 aa, synthetic construct
  REGION 1..111: note = Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, synthetic construct
  misc_feature 1..333: note = Synthetic polynucleotide
  source 1..333: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, synthetic construct
  REGION 1..218: note = Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, synthetic construct
  misc_feature 1..654: note = Synthetic polynucleotide
  source 1..654: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, synthetic construct
  REGION 1..17: note = Synthetic peptide
DIYPGQGDTSYNQKFKG

SEQ ID NO: 821 - protein, 6 aa, synthetic construct
  REGION 1..6: note = Synthetic peptide
YPGQGD

SEQ ID NO: 822 - protein, 118 aa, synthetic construct
  REGION 1..118: note = Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, synthetic construct
  misc_feature 1..354: note = Synthetic polynucleotide
  source 1..354: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, synthetic construct
  REGION 1..444: note = Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, synthetic construct
  misc_feature 1..1332: note = Synthetic polynucleotide
  source 1..1332: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, synthetic construct
  REGION 1..111: note = Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, synthetic construct
  misc_feature 1..333: note = Synthetic polynucleotide
  source 1..333: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, synthetic construct
  REGION 1..218: note = Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, synthetic construct
  misc_feature 1..654: note = Synthetic polynucleotide
  source 1..654: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, synthetic construct
  REGION 1..114: note = Synthetic polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, synthetic construct
  REGION 1..108: note = Synthetic polypeptide
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, synthetic construct
  REGION 1..120: note = Synthetic polypeptide
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, synthetic construct
  REGION 1..113: note = Synthetic polypeptide
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, synthetic construct
  REGION 1..121: note = Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, synthetic construct
  REGION 1..107: note = Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, synthetic construct
  REGION 1..451: note = Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, synthetic construct
  REGION 1..214: note = Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, synthetic construct
  misc_feature 1..363: note = Synthetic polynucleotide
  source 1..363: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, synthetic construct
  misc_feature 1..321: note = Synthetic polynucleotide
  source 1..321: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, synthetic construct
  misc_feature 1..1353: note = Synthetic polynucleotide
  source 1..1353: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, synthetic construct
  misc_feature 1..642: note = Synthetic polynucleotide
  source 1..642: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, synthetic construct
  REGION 1..5: note = Synthetic peptide
SYGVD

SEQ ID NO: 910 - protein, 7 aa, synthetic construct
  REGION 1..7: note = Synthetic peptide
GFSLSSY

SEQ ID NO: 911 - protein, 16 aa, synthetic construct
  REGION 1..16: note = Synthetic peptide
VIWGGGGTYYASSLMG

SEQ ID NO: 912 - protein, 5 aa, synthetic construct
  REGION 1..5: note = Synthetic peptide
WGGGG

SEQ ID NO: 913 - protein, 13 aa, synthetic construct
  REGION 1..13: note = Synthetic peptide
HAYGHDGGFAMDY

SEQ ID NO: 914 - protein, 11 aa, synthetic construct
  REGION 1..11: note = Synthetic peptide
RASESVSSNVA

SEQ ID NO: 915 - protein, 7 aa, synthetic construct
  REGION 1..7: note = Synthetic peptide
SESVSSN

SEQ ID NO: 916 - protein, 7 aa, synthetic construct
  REGION 1..7: note = Synthetic peptide
GASNRAT

SEQ ID NO: 917 - skipped

SEQ ID NO: 918 - protein, 9 aa, synthetic construct
  REGION 1..9: note = Synthetic peptide
GQSYSYPFT

SEQ ID NO: 919 - protein, 6 aa, synthetic construct
  REGION 1..6: note = Synthetic peptide
SYSYPF

SEQ ID NO: 920 - protein, 124 aa, synthetic construct
  REGION 1..124: note = Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, synthetic construct
  REGION 1..107: note = Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1000 - skipped (79 entries)

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, synthetic construct
  REGION 1..114: note = Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, synthetic construct
  REGION 1..297: note = Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Homo sapiens
  MOD_RES 93: note = Glu or Lys
NWVNVISDLKKIEDLIQSMHIDATLYTESDVHPSCKVTAMKCFLLELQVISLESGDASIHDTVENLIILANNSLSSNGNVTESGCKECEELEXKNIKEFLQSFVHIVQMFINTS

SEQ ID NO: 1006 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

(19) United States
(12) Patent Application Publication
(10) Pub. No.: US 2025/0025583 A1
(43) Pub. Date: Jan. 23, 2025

(54) METHOD OF TREATMENT OF NEUROENDOCRINE TUMORS

(71) Applicant: Advanced Accelerator Applications SA, Rueil-Malmaison (FR)

(72) Inventors: Donato Barbato, Ivrea (IT); Clementina Brambati, Torino (IT); Stefano Buono, Torino (IT); Daniela Chicco, Bollengo (IT); Francesco de Palo, Ivrea (IT); Lorenza Fugazza, Ivrea (IT); Maribel Lopera-Sierra, Torino (IT); Maurizio Mariani, Ivrea (IT); Giovanni Tesoriere, Noicattaro (IT)

(21) Appl. No.: 18/597,037

(22) Filed: Mar. 6, 2024

Related U.S. Application Data
(63) Continuation of application No. 17/263,140, filed on Jan. 25, 2021, now abandoned.
Application No. 17/263,140 is a continuation-in-part of application No. 16/175,239, filed on Oct. 30, 2018, now Pat. No. 10,596,278.
Application No. 16/175,239 is a continuation of application No. 16/140,962, filed on Sep. 25, 2018, now abandoned.
Application No. 16/140,962 is a continuation-in-part of application No. 16/045,484, filed on Jul. 25, 2018, now abandoned.
Application No. PCT/IB2019/056315 is a continuation-in-part of application No. 16/175,261, filed on Oct. 30, 2018, now Pat. No. 10,596,276.
Application No. 16/175,261 is a continuation-in-part of application No. 16/140,962, filed on Sep. 25, 2018, now abandoned.
(60) Provisional application No. 62/736,036, filed on Sep. 25, 2018.

(30) Foreign Application Priority Data
Jul. 25, 2018 (WO) PCT/IB2018/055575
Sep. 25, 2018 (WO) PCT/IB2018/057415

(51) Int. Cl. A61K 51/08; A61K 47/12; A61K 47/18; A61K 47/22; A61K 51/12

(52) U.S. Cl. CPC A61K 51/083; A61K 47/12; A61K 47/18; A61K 47/22; A61K 51/121

(57) ABSTRACT

The present invention relates to methods of treating cancers that overexpress somatostatin receptors, e.g. neuroendocrine tumors (NET). In particular, the invention provides novel therapies based on the combination of a peptide receptor radionuclide therapeutic (PRRT) agent and immuno-oncology (I-O) therapeutic agents, wherein said I-O therapeutic agents are selected from the group consisting of LAG-3 inhibitors, TIM-3 inhibitors, GITR angonists, TGF-β inhibitors, IL15/IL-15RA complex, and selected PD-1 inhibitors.

### SEQUENCE LISTING

The instant application contains a Sequence Listing which has been submitted electronically in ASCII format and is hereby incorporated by reference in its entirety. Said ASCII copy, created on Feb. 27, 2024, is named PAT08249-US-CNT SL.html and is 5,175,451 bytes in size.

### FIELD OF THE INVENTION

The present invention relates to methods of treating cancers that overexpress somatostatin receptors, e.g. neuroendocrine tumors (NET). In particular, the invention provides novel therapies based on the combination of a peptide receptor radionuclide therapeutic (PRRT) agent and immuno-oncology (I-O) therapeutic agents, wherein said I-O therapeutic agents are selected from the group consisting of LAG-3 inhibitors, TIM-3 inhibitors, GITR angonists, TGF-β inhibitors, IL15/IL-15RA complex, and selected PD-1 inhibitors.

### BACKGROUND

Methods of treating NET tumors based on the combination of a peptide receptor radionuclide therapeutic (PRRT) agent and immuno-oncology (I-O) therapeutic agents have been described by Buono and Sierra in WO 2016/207732, the content of which is hereby incorporated by reference.

Peptide receptor radionuclide therapy (PRRT) provides an enhanced pro-immunogenic effect with respect to correcting the immunosuppressive networks of established tumors.

Immuno-oncology (I-O) therapeutic agents defeat the established tolerance toward the cancer and recover an effective tumor-specific immune response.

The tumor-cell internal radiation provided by PRRT causes damage to the tumor and the release of tumor antigens and thus making the tumor more visible to the immune system. I-O therapy provides immune checkpoint blockade and therefore improve the immune anti-tumor T-cell response. In this way the I-O therapy enhances the effect of the internal radiation by PRRT in a synergistic way.

As NET tumors over-express somatostatin receptors, a PRRT based on somatostatin receptor binding agents, such as octreotate, is an effective targeted approach to treat such tumors.

The radionuclide lutetium-177 (177Lu) releases high energy electrons upon its beta-minus decay and has been found to effectively damage the tumor cells' DNA causing tumor cell death. The radionuclide is attached to the somatostatin receptor binding agent via a metal chelating unit, e.g. the DOTA molecule, which is covalently bound to the receptor binding agent. As an example for such a somatostatin receptor binding agent linked to a chelating unit which forms a complex with the radionuclide is ¹⁷⁷Lu-DOTA⁰-Try³-octreotate, also referred to as ¹⁷⁷Lu-DOTA-TATE or lutetium (177Lu) oxodotreotide (INN) which has become available as Lutathera.

### SUMMARY OF THE INVENTION

WO 2016/207732 describes the general therapeutic concept of the combination of PRRT and I-O therapy and provides in particular combinations with I-O therapeutic agents that inhibit the PD-1/PD-L1 and CTLA-4 pathway. The present invention provides novel combinations comprising a peptide receptor radionuclide therapeutic (PRRT) agent and one or two immuno-oncology (I-O) therapeutic agent(s) for use in treating a somatostatin receptor over-expressing cancer in a subject, wherein said I-O therapeutic agent(s) is (are) selected from the group consisting of LAG-3 inhibitors, TIM-3 inhibitors, GITR angonists, TGF-β inhibitors, IL15/IL-15RA complexes, and a improved PD-1 inhibitors, wherein said PD-1 inhibitors are selected from the group consisting of Spartalizumab, Pembrolizumab, Pidilizumab, Durvalomab, Atezolizumab, Avelumab, MEDI0680, REGN2810, TSR-042, PF-06801591, BGB-A317, BGB-108, INCSHR1210, and AMP-224.

The present inventions provides such combinations in particular with the PPRT agent lutetium (¹⁷⁷Lu) oxodotreotide and in particular for treating NET tumors.

The combinations according to the present invention may comprise one or two further anti-cancer agent(s).

In the combinations according to the present invention, the LAG-3 inhibitor may be selected from LAG525, BMS-986016, or TSR-033.

In the combinations according to the present invention, the TIM-3 inhibitor may be MBG453 or TSR-022.

In the combinations according to the present invention, the GITR agonist may be selected from GWN323, BMS-986156, MK-4166, MK-1248, TRX518, INCAGN1876, AMG 228, or INBRX-110.

In the combinations according to the present invention, the TGF-β inhibitor may be XOMA 089 or fresolimumab.

In the combinations according to the present invention, the IL-15/IL-15RA complex may be selected from NIZ985, ATL-803 or CYP0150.

Further anti-cancer agents according to the present invention may be selected in particular from the group consisting of octreotide, lanreotide, vaproreotide, pasireotide, satoreotide, everolimus, temozolomide, telotristat, sunitinib, sulfatinib, ribociclib, entinostat, and pazopanib.

Neuroendocrine tumors (NET) which may be treated by the combinations in accordance with the present invention are selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, Non-Hodgkin lymphoma, Hodgkin lymphoma, Head & Neck tumor, urothelial carcinoma (bladder), Renal Cell Carcinoma, Hepatocellular Carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, and esthesioneuroblastoma.

Further NET tumors which may be treated by the combinations according to the present invention may be selected from the group consisting of functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

In the combinations according to the present invention, the PRRT agent lutetium (¹⁷⁷Lu) oxodotreotide may be formulated as pharmaceutical aqueous solution comprising:
(a) a complex formed by
(ai) the radionuclide 177Lu (Lutetium-177) in a concentration that it provides a volumetric radioactivity of from 250 to 500 MBq/mL, and
(aii) the DOTA linked somatostatin receptor binding peptide [DOTA⁰, D-Phe¹, Tyr³]octreotate;
   (b) the stabilizers against radiolytic degradation (bi) gentisic acid in a concentration of from 0.5 to 1 mg/mL and (bii) ascorbic acid in a concentration of from 2.0 to 5.0 mg/mL;
   (c) diethylentriaminepentaacetic acid (DTPA) or a salt thereof in a concentration of from 0.01 to 0.10 mg/mL; and
   (d) an acetate buffer composed of:
(di) acetic acid in a concentration of from 0.3 to 0.7 mg/mL; and
(dii) sodium acetate in a concentration from 0.4 to 0.9 mg/mL;
preferably said acetate buffer provides for a pH of from 4.5 to 6.0, preferably from 5.0 to 5.5.

This pharmaceutical aqueous solution may be preferably prepared in such a way that gentisic acid is present during the complex formation of components (ai) and (aii) and ascorbic acid added after the complex formation of components (ai) and (aii).

The pharmaceutical aqueous solutions formulated and prepared in this way have the advantage of being highly-concentrated leading to a low infusion volume, being of high tolerability due the mild pH and absence of any ethanol, being stable with respect to chemical and radiochemical purity (≥95%) for up to 72 h when stored at room temperature (25° C.) which allows to provide this PRRT agent as ready-to-use drug product.

The present invention provides the following embodiments:

1. A combination comprising a peptide receptor radionuclide therapeutic (PRRT) agent and one or two immuno-oncology (I-O) therapeutic agent(s) for use in treating a somatostatin receptor over-expressing cancer in a subject, wherein said I-O therapeutic agent(s) is (are) selected from the group consisting of LAG-3 inhibitors, TIM-3 inhibitors, GITR angonists, TGF-β inhibitors, IL15/IL-15RA complexes, and PD-1 inhibitors, wherein said PD-1 inhibitors are selected from the group consisting of Spartalizumab, Pembrolizumab, Pidilizumab, Durvalomab, Atezolizumab, Avelumab, MEDI0680, REGN2810, TSR-042, PF-06801591, BGB-A317, BGB-108, INCSHR1210, and AMP-224.

2. A method of treating a somatostatin receptor over-expressing cancer in a subject, comprising administering to the subject a combination of a peptide receptor radionuclide therapeutic (PRRT) agent and one or two immuno-oncology (I-O) therapeutic agent(s), wherein said 1-0 therapeutic agent(s) is(are) selected from the group consisting of an LAG-3 inhibitor, a TIM-3 inhibitor, a GITR angonists, a TGF-β inhibitor, an IL15/IL-15RA complex, and a PD-1 inhibitor, wherein said PD-1 inhibitor is selected from the group consisting of Spartalizumab, Pembrolizumab, Pidilizumab, Durvalomab, Atezolizumab, Avelumab, MEDI0680, REGN2810, TSR-042, PF-06801591, BGB-A317, BGB-108, INCSHR1210, and AMP-224.

3. The combination for use of embodiment 1, or the method of embodiment 2, wherein the PRRT agent comprises the radionuclide Lutetium-177 (¹⁷⁷Lu) and a somatostatin receptor binding molecule linked to a chelating agent.

4. The combination for use or the method of embodiment 3, wherein the somatostatin receptor binding molecule is selected from the group consisting of octreotide, octreotate, lanreotide, vapreotide, pasireotide, and satoreotide.

5. The combination for use or the method of embodiment 4, wherein the chelating agent is 1,4,7,10-Tetraazacyclododecane-1,4,7,10-tetraacetic acid (DOTA).

6. The combination for use or the method of embodiment 3, wherein the somatostatin receptor binding molecule linked to the chelating agent is selected from the group consisting of DOTA-OC: [DOTA0, D-Phe1]octreotide, DOTA-TOC: [DOTA⁰, D-Phe¹, Tyr³]octreotide (i.e. edotreotide), DOTA-NOC: [DOTA⁰, D-Phe¹,1-Nal³]octreotide, DOTA-TATE: [DOTA⁰, D-Phe¹, Tyr³]octreotate (i.e. oxodotreotide), DOTA-LAN: [DOTA⁰, D-β-Nal¹]lanreotide, DOTA-VAP: [DOTA⁰, D-Phe¹, Tyr³]vapreotide, satoreotide trizoxetan, and satoreotide tetraxetan.

7. The combination for use of embodiment 1, or the method of embodiment 2, wherein the PRRT agent is lutetium (¹⁷⁷Lu) oxodotreotide (i.e. ¹⁷⁷Lu[DOTA⁰, D-Phe¹, Tyr³]octreotate).

8. The combination for use or the method of any one of embodiments 3-7, wherein the PRRT agent, e.g. lutetium (¹⁷⁷Lu) oxodotreotide or ¹⁷⁷Lu edotreotide, is formulated as a pharmaceutical aqueous solution comprising:
(a) a complex formed by
(ai) the radionuclide 177Lu (Lutetium-177) in a concentration that it provides a volumetric radioactivity of from 250 to 500 MBq/mL, and
(aii) the DOTA linked somatostatin receptor binding peptide, e.g. oxodotreotide or edotreotide;
   (b) the stabilizers against radiolytic degradation (bi) gentisic acid in a concentration of from 0.5 to 1 mg/mL and (bii) ascorbic acid in a concentration of from 2.0 to 5.0 mg/mL;
   (c) diethylentriaminepentaacetic acid (DTPA) or a salt thereof in a concentration of from 0.01 to 0.10 mg/mL; and
   (d) an acetate buffer composed of:
(di) acetic acid in a concentration of from 0.3 to 0.7 mg/mL; and
(dii) sodium acetate in a concentration from 0.4 to 0.9 mg/mL;
preferably said acetate buffer provides for a pH of from 4.5 to 6.0, preferably from 5.0 to 5.5.

9. The combination for use or the method of embodiment 8, wherein gentisic acid is present during the complex formation of components (ai) and (aii) and ascorbic acid added after the complex formation of components (ai) and (aii).

10. The combination for use of any one of embodiments 1, 3 to 9, or the method of any one of embodiments 2 to 9, wherein the LAG-3 inhibitor is chosen from LAG525, BMS-986016, or TSR-033.

11. The combination for use of any one of embodiments 1, 3 to 10, or the method of any one of embodiments 2 to 10, wherein the TIM-3 inhibitor is MBG453 or TSR-022.

12. The combination for use of any one of embodiments 1, 3 to 11, or the method of any one of embodiments 2 to 11, wherein the GITR agonist is chosen from GWN323, BMS-986156, MK-4166, MK-1248, TRX518, INCAGN1876, AMG 228, or INBRX-110.

13. The combination for use of any one of embodiments 1, 3 to 12, or the method of any one of embodiments 2 to 12, wherein the TGF-β inhibitor is XOMA 089 or fresolimumab.

14. The combination for use of any one of embodiments 1, 3 to 13, or the method of any one of embodiments 2 to 13, wherein the IL-15/IL-15RA complex is chosen from NIZ985, ATL-803 or CYP0150.

15. The combination for use of any one of embodiments 1, 3 to 14, or the method of any one of embodiments 2 to 14, comprising one or two further anti-cancer agent(s).

16. The combination for use of or the method of embodiment 15, wherein the further anti-cancer agent(s) is (are) selected from the group consisting of octreotide, lanreotide, vaproreotide, pasireotide, satoreotide, everolimus, temozolomide, telotristat, sunitinib, sulfatinib, ribociclib, entinostat, and pazopanib.

17. The combination for use of any one of embodiments 1, 3 to 16, or the method of any one of embodiments 2 to 13, wherein the somatostatin receptor over-expressing cancer is a neuroendocrine tumor (NET).

18. The combination for use of or the method of embodiment 17, wherein the neuroendocrine tumor (NET) is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, Non-Hodgkin lymphoma, Hodgkin lymphoma, Head & Neck tumor, urothelial carcinoma (bladder), Renal Cell Carcinoma, Hepatocellular Carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, and esthesioneuroblastoma.

19. The combination for use of or the method of embodiment 17, wherein the neuroendocrine tumor (NET) is selected from the group consisting of functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

The combinations described herein can provide a beneficial anti-cancer effect, e.g., an enhanced anti-cancer effect, reduced toxicity, and/or reduced side effects. For example, a first therapeutic agent, e.g., any of the therapeutic agents disclosed herein, and a second therapeutic agent, e.g., the one or more additional therapeutic agents, or all, can be administered at a lower dosage than would be required to achieve the same therapeutic effect compared to a monotherapy dose. Thus, compositions and methods for treating proliferative disorders, including cancer, using the aforesaid combination therapies are disclosed.

In some embodiments, a method of treating a subject, e.g., a subject having a cancer described herein, with a combination described herein, comprises administration of a combination as part of a therapeutic regimen. In an embodiment, a therapeutic regimen comprises one or more, e.g., two, three, or four combinations described herein. In some embodiments, the therapeutic regimen is administered to the subject in at least one phase, and optionally two phases, e.g., a first phase and a second phase. In some embodiments, the first phase comprises a dose escalation phase. In some embodiments, the first phase comprises one or more dose escalation phases, e.g., a first, second, or third dose escalation phase. In some embodiments, the dose escalation phase comprises administration of a combination comprising two, three, four, or more therapeutic agents, e.g., as described herein. In some embodiments, the second phase comprises a dose expansion phase. In some embodiments, the dose expansion phase comprises administration of a combination comprising two, three, four, or more therapeutic agents, e.g., as described herein. In some embodiments, the dose expansion phase comprises the same two, three, four, or more therapeutic agents as the dose escalation phase.

In some embodiments, the first dose escalation phase comprises administration of a combination comprising two therapeutic agents, e.g., two therapeutic agents described herein, wherein a maximum tolerated dose (MTD) or recommended dose for expansion (RDE) for one or both of the therapeutic agents of is determined. In some embodiments, prior to the first dose escalation phase, the subject was administered with one of the therapeutic agents administered in the first dose escalation phase as a single agent.

In some embodiments, the second dose escalation phase comprises administration of a combination comprising three therapeutic agents, e.g., three therapeutic agents described herein, wherein a maximum tolerated dose (MTD) or recommended dose for expansion (RDE) for one, two, or all of the therapeutic agents is determined. In some embodiments, the second dose escalation phase starts after the first dose escalation phase ends. In some embodiments, the second dose escalation phase comprises administration of one or more of the therapeutic agents administered in the first dose escalation phase. In some embodiments, the second dose escalation phase is performed without performing the first dose escalation phase.

In some embodiments, the third dose escalation phase comprises administration of a combination comprising four therapeutic agents, e.g., four therapeutic agents described herein, wherein a maximum tolerated dose (MTD) or recommended dose for expansion (RDE) of one, two, three, or all of the therapeutic agents is determined. In some embodiments, the third dose escalation phase starts after the first or second dose escalation phase ends. In some embodiments, the third dose escalation phase comprises administration of one or more (e.g., all) of therapeutic agents administered in the second dose escalation phase. In some embodiments, the third dose escalation phase comprises administration of one or more of the therapeutic agents administered in the first dose escalation phase. In some embodiments, the third dose escalation phase is performed without performing the first, second, or both dose escalation phases.

In some embodiments, the dose expansion phase starts after the first, second or third dose escalation phase ends. In some embodiments, the dose expansion phase comprises administration of a combination administered in the dose escalation phase, e.g., the first, second, or third dose escalation phase. In an embodiment, a biopsy is obtained from a subject in the dose expansion phase. In an embodiment, the subject is treated for a NET tumor, e.g., SCLC.

Without wishing to be bound by theory, it is believed that in some embodiments, a therapeutic regimen comprising a dose escalation phase and a dose expansion phase allows for entry of new agents or regiments for combination, rapid generation of combinations, and/or assessment of safety and activity of tolerable combinations.

Additional features or embodiments of the methods, compositions, dosage formulations, and kits described herein include one or more of the following.

### DETAILED DESCRIPTION

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, e.g., the anti-PD-1 antibody molecule, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., an activity of a given molecule, e.g., an inhibitory molecule, of at least 5%, 10%, 20%, 30%, 40% or more is included by this term. Thus, inhibition need not be 100%.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

As used herein, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The compositions and methods of the present invention encompass polypeptides and nucleic acids having the sequences specified, or sequences substantially identical or similar thereto, e.g., sequences at least 85%, 90%, 95%, 96%, 97%, 98%, 99% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Antibody Molecules

In one embodiment, a combination described herein comprises a therapeutic agent which is an antibody molecule.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

An antibody molecules may be conjugated to another molecular entity, typically a label or a therapeutic (e.g., a cytotoxic or cytostatic) agent or moiety. Radioactive isotopes can be used in diagnostic or therapeutic applications. Radioactive isotopes that can be coupled to the anti-PSMA antibodies include, but are not limited to α-, β-, or γ-emitters, or β- and γ-emitters. Such radioactive isotopes include, but are not limited to iodine (¹³¹I or ¹²⁵I) yttrium (⁹⁰Y), lutetium (⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹² Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. The conjugated antibody is radiolabeled with a radioisotope, e.g., 11Indium, 90Yttrium and 177Lutetium, to thereby produce a labeled antibody molecule.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Multispecific Antibody Molecules

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

### Immuno-Oncology Therapeutic Agents

### Selected PD-1 Inhibitors

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

In certain embodiments, a combination described herein comprises a PD-1 inhibitor. In some embodiments, the PD-1 inhibitor is chosen from PDR001 (Novartis), Pembrolizumab (Merck & Co), Pidilizumab (CureTech), Durvalomab, Atezolizumab, Avelumab, MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In some embodiments, the anti-PD-1 antibody molecule is Spartalizumab (PDR001).

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 1
Amino acid and nucleotide sequences of
exemplary anti-PD-1 antibody molecules

| | | |
|---|---|---|
| BAP049-Clone-B HC | | |
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRSE |
| | | DTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |
| BAP049-Clone-B LC | | |
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTWYQQKP |
| | | GKAPKLLIYWASTRESGVPSRFSGSGSGTDFTFTISSLQPEDIATY |
| | | YCQNDYSYPYTFGQGTKVEIK |
| SEQ ID NO: 517 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAG |
| SEQ ID NO: 518 | Light | EIVLTQSPATLSLSPGERATLSCKSSQSLLDSGNQKNFLTWYQQKP |
| | chain | GKAPKLLIYWASTRESGVPSRFSGSGSGTDFTFTISSLQPEDIATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 519 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAGCGTACGGT |
| | | GGCCGCTCCCAGCGTGTTCATCTTCCCCCCCAGCGACGAGCAGC |
| | | TGAAGAGCGGCACCGCCAGCGTGGTGTGCCTGCTGAACAACTT |
| | | CTACCCCCGGGAGGCCAAGGTGCAGTGGAAGGTGGACAACGCC |
| | | CTGCAGAGCGGCAACAGCCAGGAGAGCGTCACCGAGCAGGAC |
| | | AGCAAGGACTCCACCTACAGCCTGAGCAGCACCCTGACCCTGA |
| | | GCAAGGCCGACTACGAGAAGCATAAGGTGTACGCCTGCGAGGT |
| | | GACCCACCAGGGCCTGTCCAGCCCCGTGACCAAGAGCTTCAAC |
| | | AGGGGCGAGTGC |
| BAP049-Clone-E HC | | |
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRS |
| | | EDTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCP |
| | | PCPAPEFLGGPSVFLFPPKPKDTLMISRTPEVTCVVVDVSQEDPE |
| | | VQFNWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |
| BAP049-Clone-E LC | | |
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWYQQKP |
| | | GQAPRLLIYWASTRESGVPSRFSGSGSGTDFTFTISSLEAEDAATY |
| | | YCQNDYSYPYTFGQGTKVEIK |
| SEQ ID NO: 521 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAG |
| SEQ ID NO: 522 | Light | EIVLTQSPATLSLSPGERATLSCKSSQSLLDSGNQKNFLTWYQQKP |
| | chain | GQAPRLLIYWASTRESGVPSRFSGSGSGTDFTFTISSLEAEDAATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 523 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAGCGTACGGTG |
| | | GCCGCTCCCAGCGTGTTCATCTTCCCCCCCAGCGACGAGCAGCT |
| | | GAAGAGCGGCACCGCCAGCGTGGTGTGCCTGCTGAACAACTTC |
| | | TACCCCCGGGAGGCCAAGGTGCAGTGGAAGGTGGACAACGCCC |
| | | TGCAGAGCGGCAACAGCCAGGAGAGCGTCACCGAGCAGGACA |
| | | GCAAGGACTCCACCTACAGCCTGAGCAGCACCCTGACCCTGAG |
| | | CAAGGCCGACTACGAGAAGCATAAGGTGTACGCCTGCGAGGTG |
| | | ACCCACCAGGGCCTGTCCAGCCCCGTGACCAAGAGCTTCAACA |
| | | GGGGCGAGTGC |
| BAP049-Clone-B HC | | |
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat) | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat) | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

In some embodiments, the PD-1 inhibitor is administered at a dose of about 200 mg to about 500 mg (e.g., about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g., about 300 mg) once every 3 weeks. In yet other embodiments, the PD-1 inhibitor is administered at a dose of about 300 mg to about 500 mg (e.g., about 400 mg) once every 4 weeks.

In some embodiments, the combination comprises a PD-1 inhibitor, e.g., PDR001, and a TGF-β inhibitor, e.g., NIS793. In some embodiments, this combination is administered to a subject in a therapeutically effective amount to treat, e.g., a pancreatic cancer.

In some embodiments, the combination comprises a PD-1 inhibitor, e.g., PDR001, and a TLR7 agonist, e.g., LHC165. In some embodiments, this combination is administered to a subject in a therapeutically effective amount to treat, e.g., a pancreatic cancer. In some embodiments, the TLR7 agonist, e.g., LHC165 is administered via intra-tumoral injection.

In some embodiments, the combination comprises a PD-1 inhibitor, e.g., PDR001, and an adenosine receptor antagonist, e.g., PBF509 (NIR178). In some embodiments, this combination is administered to a subject in a therapeutically effective amount to treat, e.g., a pancreatic cancer.

In some embodiments, the combination comprises a PD-1 inhibitor, e.g., PDR001, and an inhibitor of Porcupine, e.g., WNT974. In some embodiments, this combination is administered to a subject in a therapeutically effective amount to treat, e.g., a pancreatic cancer.

In some embodiments, the combination comprises a PD-1 inhibitor, e.g., PDR001, and an A2aR antagonist, e.g., PBF509 (NIR178). In some embodiments, this combination is administered to a subject in a therapeutically effective amount to treat, e.g., a CRC or gastric cancer. Without wishing to be bound by theory, it is believed that a combination comprising a PD-1 inhibitor, e.g., PDR001, and an A2aR antagonist, e.g., PBF509 (NIR178), can result in increased efficacy of the anti-PD-1 inhibitor. In some embodiments, the combination of a PD-1 inhibitor, e.g., PDR001, and an A2aR antagonist, e.g., PBF509 (NIR178), results in regression of a CRC tumor.

In some embodiments, the combination comprises a PD-1 inhibitor, e.g., PDR001, and a PD-L1 inhibitor, e.g., FAZ053. In some embodiments, the combination is administered to a subject in a therapeutically effective amount to treat, e.g., a breast cancer, e.g., a triple negative breast cancer.

### Other Exemplary PD-1 Inhibitors

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 2.

In one embodiment, the anti-PD-1 antibody molecule is Durvalomab.

In one embodiment, the anti-PD-1 antibody molecule is Atezolizumab.

In one embodiment, the anti-PD-1 antibody molecule is Avelumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 2
Amino acid sequences of other exemplary anti-PD-1 antibody molecules

| | | |
|---|---|---|
| Pembrolizumab | | |
| SEQ ID NO: 537 | Heavy | QVQLVQSGVEVKKPGASVKVSCKASGYTFTNYYMYWVRQAPGQGLE |
| | chain | WMGGINPSNGGTNFNEKFKNRVTLTTDSSTTTAYMELKSLQFDDTA |
| | | VYYCARRDYRFDMGFDYWGQGTTVTVSSASTKGPSVFPLAPCSRST |
| | | SESTAALGCLVKDYFPEPVTVSWNSGALTSGVHTFPAVLQSSGLYS |
| | | LSSVVTVPSSSLGTKTYTCNVDHKPSNTKVDKRVESKYGPPCPPCP |
| | | APEFLGGPSVFLFPPKPKDTLMISRTPEVTCVVVDVSQEDPEVQFN |
| | | WYVDGVEVHNAKTKPREEQFNSTYRVVSVLTVLHQDWLNGKEYKCK |
| | | VSNKGLPSSIEKTISKAKGQPREPQVYTLPPSQEEMTKNQVSLTCL |
| | | VKGFYPSDIAVEWESNGQPENNYKTTPPVLDSDGSFFLYSRLTVDK |
| | | SRWQEGNVFSCSVMHEALHNHYTQKSLSLSLGK |
| SEQ ID NO: 538 | Light | EIVLTQSPATLSLSPGERATLSCRASKGVSTSGYSYLHWYQQKPGQA |
| | chain | PRLLIYLASYLESGVPARFSGSGSGTDFTLTISSLEPEDFAVYYCQH |
| | | SRDLPLTFGGGTKVEIKRTVAAPSVFIFPPSDEQLKSGTASVVCLLN |
| | | NFYPREAKVQWKVDNALQSGNSQESVTEQDSKDSTYSLSSTLTLSKA |
| | | DYEKHKVYACEVTHQGLSSPVTKSFNRGEC |
| Pidilizumab | | |
| SEQ ID NO: 539 | Heavy | QVQLVQSGSELKKPGASVKISCKASGYTFTNYGMNWVRQAPGQGLQ |
| | chain | WMGWINTDSGESTYAEEFKGRFVFSLDTSVNTAYLQITSLTAEDTGM |
| | | YFCVRVGYDALDYWGQGTLVTVSSASTKGPSVFPLAPSSKSTSGGTA |
| | | ALGCLVKDYFPEPVTVSWNSGALTSGVHTFPAVLQSSGLYSLSSVVT |
| | | VPSSSLGTQTYICNVNHKPSNTKVDKRVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGK |
| SEQ ID NO: 540 | Light | EIVLTQSPSSLSASVGDRVTITCSARSSVSYMHWFQQKPGKAPKLWIY |
| | chain | RTSNLASGVPSRFSGSGSGTSYCLTINSLQPEDFATYYCQQRSSFPLT |
| | | FGGGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVY |
| | | ACEVTHQGLSSPVTKSFNRGEC |

### Additional Combination Therapies

In an embodiment, the combination comprises a PD-1 inhibitor (e.g., PDR001), and an mTOR inhibitor, e.g., RAD001 (also known as everolimus). In some embodiments, the combination comprises PDR001 and an mTOR inhibitor, e.g., RAD001. In some embodiments, the combination comprises PDR001 and RAD001. In some embodiments, the mTOR inhibitor, e.g., RAD001, is administered once weekly at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once weekly at a dose of 10 mg. In some embodiments, the mTOR inhibitor, e.g., RAD001, is administered once weekly at a dose of 5 mg. In some embodiments, the mTOR inhibitor, e.g., RAD001, is administered once daily at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once daily at a dose of 0.5 mg. In some embodiments, this combination is administered to a subject in a therapeutically effective amount to treat a cancer, e.g., a cancer described herein, e.g., a colorectal cancer.

### LAG-3 Inhibitors

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP050-Clone I or BAP050-Clone J disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 5.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 5
Amino acid and nucleotide sequences of exemplary
anti-LAG-3 antibody molecules

| | | |
|---|---|---|
| BAP050-Clone I HC | | |
| SEQ ID NO: 701 (Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO:706 | VH | QVQLVQSGAEVKKPGASVKVSCKASGFTLTNYGMNWVRQ |
| | | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSS |
| SEQ ID NO: 707 | DNA VH | CAAGTGCAGCTGGTGCAGTCGGGAGCCGAAGTGAAGAAG |
| | | CCTGGAGCCTCGGTGAAGGTGTCGTGCAAGGCATCCGGA |
| | | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGC |
| SEQ ID NO: 708 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 709 | Heavy | QVQLVQSGAEVKKPGASVKVSCKASGFTLTNYGMNWVRQ |
| | chain | ARGQRLEWIGWINTDTGEPTYADDFKGRFVFSLDTSVSTAY |
| | | LQISSLKAEDTAVYYCARNPPYYYGTNNAEAMDYWGQGTT |
| | | VTVSSASTKGPSVFPLAPCSRSTSESTAALGCLVKDYFPEPV |
| | | TVSWNSGALTSGVHTFPAVLQSSGLYSLSSVVTVPSSSLGTK |
| | | TYTCNVDHKPSNTKVDKRVESKYGPPCPPCPAPEFLGGPSV |
| | | FLFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKNQV |
| | | SLTCLVKGFYPSDIAVEWESNGQPENNYKTTPPVLDSDGSFF |
| | | LYSRLTVDKSRWQEGNVFSCSVMHEALHNHYTQKSLSLSL |
| | | G |
| SEQ ID NO: 716 | DNA | CAAGTGCAGCTGGTGCAGTCGGGAGCCGAAGTGAAGAAG |
| | heavy | CCTGGAGCCTCGGTGAAGGTGTCGTGCAAGGCATCCGGA |
| | chain | TTCACCCTCACCAATTACGGGATGAACTGGGTCAGACAG |
| | | GCCCGGGGTCAACGGCTGGAGTGGATCGGATGGATTAAC |
| | | ACCGACACCGGGGAGCCTACCTACGCGGACGATTTCAAG |
| | | GGACGGTTCGTGTTCTCCCTCGACACCTCCGTGTCCACCG |
| | | CCTACCTCCAAATCTCCTCACTGAAAGCGGAGGACACCG |
| | | CCGTGTACTATTGCGCGAGGAACCCGCCCTACTACTACGG |
| | | AACCAACAACGCCGAAGCCATGGACTACTGGGGCCAGGG |
| | | CACCACTGTGACTGTGTCCAGCGCGTCCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 717 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCAGGGGCCAGCGGCTGGAATGGATCGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |
| BAP050-Clone I LC | | |
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL | DIQMTQSPSSLSASVGDRVTITCSSSQDISNYLNWYLQKPGQ |
| | | SPQLLIYYTSTLHLGVPSRFSGSGSGTEFTLTISSLQPDDFA |
| | | TYYCQQYYNLPWTFGQGTKVEIK |
| SEQ ID NO: 719 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTA |
| | | AG |
| SEQ ID NO: 720 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAG |
| SEQ ID NO: 721 | Light | DIQMTQSPSSLSASVGDRVTITCSSSQDISNYLNWYLQKPGQS |
| | chain | PQLLIYYTSTLHLGVPSRFSGSGSGTEFTLTISSLQPDDFATY |
| | | YCQQYYNLPWTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| SEQ ID NO: 722 | DNA light | GATATTCAGATGACTCAGTCACCTAGTAGCCTGAGCGCTA |
| | chain | GTGTGGGCGATAGAGTGACTATCACCTGTAGCTCTAGTCA |
| | | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTA |
| | | AGCGTACGGTGGCCGCTCCCAGCGTGTTCATCTTCCCCCC |
| | | CAGCGACGAGCAGCTGAAGAGCGGCACCGCCAGCGTGGT |
| | | GTGCCTGCTGAACAACTTCTACCCCCGGGAGGCCAAGGT |
| | | GCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 723 | DNA light | GACATCCAGATGACCCAGTCCCCCTCCAGCCTGTCTGCTT |
| | chain | CCGTGGGCGACAGAGTGACCATCACCTGTTCCTCCAGCC |
| | | AGGACATCTCCAACTACCTGAACTGGTATCTGCAGAAGC |
| | | CCGGCCAGTCCCCTCAGCTGCTGATCTACTACACCTCCAC |
| | | CCTGCACCTGGGCGTGCCCTCCAGATTTTCCGGCTCTGGC |
| | | TCTGGCACCGAGTTTACCCTGACCATCAGCTCCCTGCAGC |
| | | CCGACGACTTCGCCACCTACTACTGCCAGCAGTACTACAA |
| | | CCTGCCCTGGACCTTCGGCCAGGGCACCAAGGTGGAAAT |
| | | CAAGCGTACGGTGGCCGCTCCCAGCGTGTTCATCTTCCCC |
| | | CCAAGCGACGAGCAGCTGAAGAGCGGCACCGCCAGCGTG |
| | | GTGTGTCTGCTGAACAACTTCTACCCCAGGGAGGCCAAG |
| | | GTGCAGTGGAAGGTGGACAACGCCCTGCAGAGCGGCAAC |
| | | AGCCAGGAGAGCGTCACCGAGCAGGACAGCAAGGACTC |
| | | CACCTACAGCCTGAGCAGCACCCTGACCCTGAGCAAGGC |
| | | CGACTACGAGAAGCACAAGGTGTACGCCTGTGAGGTGAC |
| | | CCACCAGGGCCTGTCCAGCCCCGTGACCAAGAGCTTCAA |
| | | CAGGGGCGAGTGC |
| BAP050-Clone J HC | | |
| SEQ ID NO: 701 (Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQ |
| | | APGQGLEWMGWINTDTGEPTYADDFKGRFVFSLDTSVSTA |
| | | YLQISSLKAEDTAVYYCARNPPYYYGTNNAEAMDYWGQG |
| | | TTVTVSS |
| SEQ ID NO: 725 | DNA VH | CAGGTGCAGCTGGTGCAGTCAGGCGCCGAAGTGAAGAAA |
| | | CCCGGCGCTAGTGTGAAAGTCAGCTGTAAAGCTAGTGGC |
| | | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGC |
| SEQ ID NO: 726 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGCT |
| | | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCT |
| SEQ ID NO: 727 | Heavy | QVQLVQSGAEVKKPGASVKVSCKASGFTLTNYGMNWVRQ |
| | chain | APGQGLEWMGWINTDTGEPTYADDFKGRFVFSLDTSVSTA |
| | | YLQISSLKAEDTAVYYCARNPPYYYGTNNAEAMDYWGQGT |
| | | TVTVSSASTKGPSVFPLAPCSRSTSESTAALGCLVKDYFPEP |
| | | VTVSWNSGALTSGVHTFPAVLQSSGLYSLSSVVTVPSSSLGT |
| | | KTYTCNVDHKPSNTKVDKRVESKYGPPCPPCPAPEFLGGPS |
| | | VFLFPPKPKDTLMISRTPEVTCVVVDVSQEDPEVQFNWYVD |
| | | GVEVHNAKTKPREEQFNSTYRVVSVLTVLHQDWLNGKEYK |
| | | CKVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSLS |
| | | LG |
| SEQ ID NO: 728 | DNA | CAGGTGCAGCTGGTGCAGTCAGGCGCCGAAGTGAAGAAA |
| | heavy | CCCGGCGCTAGTGTGAAAGTCAGCTGTAAAGCTAGTGGC |
| | chain | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACCG |
| | | CCTACCTGCAGATCTCTAGCCTGAAGGCCGAGGACACCG |
| | | CCGTCTACTACTGCGCTAGAAACCCCCCCTACTACTACGG |
| | | CACTAACAACGCCGAGGCTATGGACTACTGGGGTCAAGG |
| | | CACTACCGTGACCGTGTCTAGCGCTAGCACTAAGGGCCC |
| | | GTCCGTGTTCCCCCTGGCACCTTGTAGCCGGAGCACTAGC |
| | | GAATCCACCGCTGCCCTCGGCTGCCTGGTCAAGGATTACT |
| | | TCCCGGAGCCCGTGACCGTGTCCTGGAACAGCGGAGCCC |
| | | TGACCTCCGGAGTGCACACCTTCCCCGCTGTGCTGCAGAG |
| | | CTCCGGGCTGTACTCGCTGTCGTCGGTGGTCACGGTGCCT |
| | | TCATCTAGCCTGGGTACCAAGACCTACACTTGCAACGTGG |
| | | ACCACAAGCCTTCCAACACTAAGGTGGACAAGCGCGTCG |
| | | AATCGAAGTACGGCCCACCGTGCCCGCCTTGTCCCGCGCC |
| | | GGAGTTCCTCGGCGGTCCCTCGGTCTTTCTGTTCCCACCG |
| | | AAGCCCAAGGACACTTTGATGATTTCCCGCACCCCTGAA |
| | | GTGACATGCGTGGTCGTGGACGTGTCACAGGAAGATCCG |
| | | GAGGTGCAGTTCAATTGGTACGTGGATGGCGTCGAGGTG |
| | | CACAACGCCAAAACCAAGCCGAGGGAGGAGCAGTTCAA |
| | | CTCCACTTACCGCGTCGTGTCCGTGCTGACGGTGCTGCAT |
| | | CAGGACTGGCTGAACGGGAAGGAGTACAAGTGCAAAGT |
| | | GTCCAACAAGGGACTTCCTAGCTCAATCGAAAAGACCAT |
| | | CTCGAAAGCCAAGGGACAGCCCCGGGAACCCCAAGTGTA |
| | | TACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGA |
| | | TGCATGAAGCCCTGCACAACCACTACACTCAGAAGTCCC |
| | | TGTCCCTCTCCCTGGGA |
| SEQ ID NO: 729 | DNA | CAGGTGCAGCTGGTGCAGTCTGGCGCCGAAGTGAAGAAA |
| | heavy | CCTGGCGCCTCCGTGAAGGTGTCCTGCAAGGCCTCTGGCT |
| | chain | TCACCCTGACCAACTACGGCATGAACTGGGTGCGACAGG |
| | | CCCCTGGACAGGGCCTGGAATGGATGGGCTGGATCAACA |
| | | CCGACACCGGCGAGCCTACCTACGCCGACGACTTCAAGG |
| | | GCAGATTCGTGTTCTCCCTGGACACCTCCGTGTCCACCGC |
| | | CTACCTGCAGATCTCCAGCCTGAAGGCCGAGGATACCGC |
| | | CGTGTACTACTGCGCCCGGAACCCCCCTTACTACTACGGC |
| | | ACCAACAACGCCGAGGCCATGGACTATTGGGGCCAGGGC |
| | | ACCACCGTGACCGTGTCCTCTGCTTCTACCAAGGGGCCCA |
| | | GCGTGTTCCCCCTGGCCCCCTGCTCCAGAAGCACCAGCGA |
| | | GAGCACAGCCGCCCTGGGCTGCCTGGTGAAGGACTACTT |
| | | CCCCGAGCCCGTGACCGTGTCCTGGAACAGCGGAGCCCT |
| | | GACCAGCGGCGTGCACACCTTCCCCGCCGTGCTGCAGAG |
| | | CAGCGGCCTGTACAGCCTGAGCAGCGTGGTGACCGTGCC |
| | | CAGCAGCAGCCTGGGCACCAAGACCTACACCTGTAACGT |
| | | GGACCACAAGCCCAGCAACACCAAGGTGGACAAGAGGG |
| | | TGGAGAGCAAGTACGGCCCACCCTGCCCCCCCTGCCCAG |
| | | CCCCCGAGTTCCTGGGCGGACCCAGCGTGTTCCTGTTCCC |
| | | CCCCAAGCCCAAGGACACCCTGATGATCAGCAGAACCCC |
| | | CGAGGTGACCTGTGTGGTGGTGGACGTGTCCCAGGAGGA |
| | | CCCCGAGGTCCAGTTCAACTGGTACGTGGACGGCGTGGA |
| | | GGTGCACAACGCCAAGACCAAGCCCAGAGAGGAGCAGTT |
| | | TAACAGCACCTACCGGGTGGTGTCCGTGCTGACCGTGCTG |
| | | CACCAGGACTGGCTGAACGGCAAAGAGTACAAGTGTAAG |
| | | GTCTCCAACAAGGGCCTGCCAAGCAGCATCGAAAAGACC |
| | | ATCAGCAAGGCCAAGGGCCAGCCTAGAGAGCCCCAGGTC |
| | | TACACCCTGCCACCCAGCCAAGAGGAGATGACCAAGAAC |
| | | CAGGTGTCCCTGACCTGTCTGGTGAAGGGCTTCTACCCAA |
| | | GCGACATCGCCGTGGAGTGGGAGAGCAACGGCCAGCCCG |
| | | AGAACAACTACAAGACCACCCCCCCAGTGCTGGACAGCG |
| | | ACGGCAGCTTCTTCCTGTACAGCAGGCTGACCGTGGACA |
| | | AGTCCAGATGGCAGGAGGGCAACGTCTTTAGCTGCTCCG |
| | | TGATGCACGAGGCCCTGCACAACCACTACACCCAGAAGA |
| | | GCCTGAGCCTGTCCCTGGGC |
| BAP050-Clone J LC | | |
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKPGK |
| | | APKLLIYYTSTLHLGIPPRFSGSGYGTDFTLTINNIESEDAAY |
| | | YFCQQYYNLPWTFGQGTKVEIK |
| SEQ ID NO: 731 | DNA VL | GATATTCAGATGACTCAGTCACCTAGTAGCCTGAGCGCTA |
| | | GTGTGGGCGATAGAGTGACTATCACCTGTAGCTCTAGTCA |
| | | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAG |
| SEQ ID NO: 732 | DNA VL | GACATCCAGATGACCCAGTCCCCCTCCAGCCTGTCTGCTT |
| | | CCGTGGGCGACAGAGTGACCATCACCTGTTCCTCCAGCC |
| | | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAG |
| SEQ ID NO: 733 | Light | DIQMTQSPSSLSASVGDRVTITCSSSQDISNYLNWYQQKPGK |
| | chain | APKLLIYYTSTLHLGIPPRFSGSGYGTDFTLTINNIESEDAAY |
| | | YFCQQYYNLPWTFGQGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| SEQ ID NO: 734 | DNA light | GATATTCAGATGACTCAGTCACCTAGTAGCCTGAGCGCTA |
| | chain | GTGTGGGCGATAGAGTGACTATCACCTGTAGCTCTAGTCA |
| | | GGATATCTCTAACTACCTGAACTGGTATCAGCAGAAGCC |
| | | CGGTAAAGCCCCTAAGCTGCTGATCTACTACACTAGCACC |
| | | CTGCACCTGGGAATCCCCCCTAGGTTTAGCGGTAGCGGCT |
| | | ACGGCACCGACTTCACCCTGACTATTAACAATATCGAGTC |
| | | AGAGGACGCCGCCTACTACTTCTGTCAGCAGTACTATAAC |
| | | CTGCCCTGGACCTTCGGTCAAGGCACTAAGGTCGAGATT |
| | | AAGCGTACGGTGGCCGCTCCCAGCGTGTTCATCTTCCCCC |
| | | CCAGCGACGAGCAGCTGAAGAGCGGCACCGCCAGCGTGG |
| | | TGTGCCTGCTGAACAACTTCTACCCCCGGGAGGCCAAGG |
| | | TGCAGTGGAAGGTGGACAACGCCCTGCAGAGCGGCAACA |
| | | GCCAGGAGAGCGTCACCGAGCAGGACAGCAAGGACTCC |
| | | ACCTACAGCCTGAGCAGCACCCTGACCCTGAGCAAGGCC |
| | | GACTACGAGAAGCATAAGGTGTACGCCTGCGAGGTGACC |
| | | CACCAGGGCCTGTCCAGCCCCGTGACCAAGAGCTTCAAC |
| | | AGGGGCGAGTGC |
| SEQ ID NO: 735 | DNA light | GACATCCAGATGACCCAGTCCCCCTCCAGCCTGTCTGCTT |
| | chain | CCGTGGGCGACAGAGTGACCATCACCTGTTCCTCCAGCC |
| | | AGGACATCTCCAACTACCTGAACTGGTATCAGCAGAAGC |
| | | CCGGCAAGGCCCCCAAGCTGCTGATCTACTACACCTCCAC |
| | | CCTGCACCTGGGCATCCCCCCTAGATTCTCCGGCTCTGGC |
| | | TACGGCACCGACTTCACCCTGACCATCAACAACATCGAG |
| | | TCCGAGGACGCCGCCTACTACTTCTGCCAGCAGTACTACA |
| | | ACCTGCCCTGGACCTTCGGCCAGGGCACCAAGGTGGAAA |
| | | TCAAGCGTACGGTGGCCGCTCCCAGCGTGTTCATCTTCCC |
| | | CCCAAGCGACGAGCAGCTGAAGAGCGGCACCGCCAGCGT |
| | | GGTGTGTCTGCTGAACAACTTCTACCCCAGGGAGGCCAA |
| | | GGTGCAGTGGAAGGTGGACAACGCCCTGCAGAGCGGCAA |
| | | CAGCCAGGAGAGCGTCACCGAGCAGGACAGCAAGGACT |
| | | CCACCTACAGCCTGAGCAGCACCCTGACCCTGAGCAAGG |
| | | CCGACTACGAGAAGCACAAGGTGTACGCCTGTGAGGTGA |
| | | CCCACCAGGGCCTGTCCAGCCCCGTGACCAAGAGCTTCA |
| | | ACAGGGGCGAGTGC |
| BAP050-Clone I HC | | |
| SEQ ID NO: 736 (Kabat) | HCDR1 | AATTACGGGATGAAC |
| SEQ ID NO: 737 (Kabat) | HCDR1 | AACTACGGCATGAAC |
| SEQ ID NO: 738 (Kabat) | HCDR2 | TGGATTAACACCGACACCGGGGAGCCTACCTACGCGGAC |
| | | GATTTCAAGGGA |
| SEQ ID NO: 739 (Kabat) | HCDR2 | TGGATCAACACCGACACCGGCGAGCCTACCTACGCCGAC |
| | | GACTTCAAGGGC |
| SEQ ID NO: 740 (Kabat) | HCDR3 | AACCCGCCCTACTACTACGGAACCAACAACGCCGAAGCC |
| | | ATGGACTAC |
| SEQ ID NO: 741 (Kabat) | HCDR3 | AACCCCCCTTACTACTACGGCACCAACAACGCCGAGGCC |
| | | ATGGACTAT |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGCC |
| (Chothia) | | ATGGACTAC |
| SEQ ID NO: 741 | HCDR3 | AACCCCCCTTACTACTACGGCACCAACAACGCCGAGGCC |
| (Chothia) | | ATGGACTAT |
| BAP050-Clone I LC | | |
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQ ID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone J HC | | |
| SEQ ID NO: 758 (Kabat) | HCDR1 | AACTACGGGATGAAC |
| SEQ ID NO: 737 (Kabat) | HCDR1 | AACTACGGCATGAAC |
| SEQ ID NO: 759 (Kabat) | HCDR2 | TGGATTAACACCGACACCGGCGAGCCTACCTACGCCGAC |
| | | GACTTTAAGGGC |
| SEQ ID NO: 739 (Kabat) | HCDR2 | TGGATCAACACCGACACCGGCGAGCCTACCTACGCCGAC |
| | | GACTTCAAGGGC |
| SEQ ID NO: 760 (Kabat) | HCDR3 | AACCCCCCCTACTACTACGGCACTAACAACGCCGAGGCT |
| | | ATGGACTAC |
| SEQ ID NO: 741 (Kabat) | HCDR3 | AACCCCCCTTACTACTACGGCACCAACAACGCCGAGGCC |
| | | ATGGACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGCT |
| (Chothia) | | ATGGACTAC |
| SEQ ID NO: 741 | HCDR3 | AACCCCCCTTACTACTACGGCACCAACAACGCCGAGGCC |
| (Chothia) | | ATGGACTAT |
| BAP050-Clone J LC | | |
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQ ID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

In some embodiments, the LAG-3 inhibitor (e.g., an anti-LAG-3 antibody molecule described herein) is administered at a dose of about 300-1000 mg, e.g., about 300 mg to about 500 mg, about 400 mg to about 800 mg, or about 700 mg to about 900 mg. In embodiments, the LAG-3 inhibitor is administered once every week, once every two weeks, once every three weeks, once every four weeks, once every five weeks or once every six weeks. In embodiments, the LAG-3 inhibitor is administered once every 3 weeks. In embodiments, the LAG-3 inhibitor is administered once every 4 weeks. In other embodiments, the LAG-3 inhibitor is administered at a dose of about 300 mg to about 500 mg (e.g., about 400 mg) once every 3 weeks. In yet other embodiments, the PD-1 inhibitor is administered at a dose of about 700 mg to about 900 mg (e.g., about 800 mg) once every 4 weeks. In yet other embodiments, the LAG-3 inhibitor is administered at a dose of about 400 mg to about 800 mg (e.g., about 600 mg) once every 4 weeks.

In some embodiments, a composition comprises a LAG-3 inhibitor, e.g., a LAG-3 inhibitor described herein, and a PD-1 inhibitor, e.g., a PD-1 inhibitor described herein. In some embodiments, the combination of a LAG-3 inhibitor and a PD-1 inhibitor is administered in a therapeutically effective amount to a subject with a solid tumor, e.g., a breast cancer, e.g., a triple negative breast cancer. Without wishing to be bound by theory, it is believed that a combination comprising a LAG-3 inhibitor and a PD-1 inhibitor has increased activity compared to administration of a PD-1 inhibitor alone.

In some embodiments, a composition comprises a LAG-3 inhibitor, e.g., a LAG-3 inhibitor described herein, a GITR agonist, e.g., a GITR agonist described herein, and a PD-1 inhibitor, e.g., a PD-1 inhibitor described herein. In some embodiments, the combination of a LAG-3 inhibitor, a GITR agonist, and a PD-1 inhibitor is administered in a therapeutically effective amount to a subject with a solid tumor, e.g., a breast cancer, e.g., a triple negative breast cancer. In some embodiments, a combination comprising a LAG-3 inhibitor, a GITR agonist, and a PD-1 inhibitor can result in increased IL-2 production.

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 6.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 6. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 6
Amino acid sequences of other exemplary anti-LAG-3 antibody molecules

| | | |
|---|---|---|
| BMS-986016 | | |
| SEQ ID NO: 762 | Heavy chain | QVQLQQWGAGLLKPSETLSLTCAVYGGSFSDYYWNWIRQPPGK |
| | | GLEWIGEINHRGSTNSNPSLKSRVTLSLDTSKNQFSLKLRSVTAA |
| | | DTAVYYCAFGYSDYEYNWFDPWGQGTLVTVSSASTKGPSVFPL |
| | | APCSRSTSESTAALGCLVKDYFPEPVTVSWNSGALTSGVHTFPA |
| | | VLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTV |
| | | LHQDWLNGKEYKCKVSNKGLPSSIEKTISKAKGQPREPQVYTLP |
| | | PSQEEMTKNQVSLTCLVKGFYPSDIAVEWESNGQPENNYKTTPP |
| | | VLDSDGSFFLYSRLTVDKSRWQEGNVFSCSVMHEALHNHYTQK |
| | | SLSLSLGK |
| SEQ ID NO: 763 | Light chain | EIVLTQSPATLSLSPGERATLSCRASQSISSYLAWYQQKPGQAPR |
| | | LLIYDASNRATGIPARFSGSGSGTDFTLTISSLEPEDFAVYYCQQR |
| | | SNWPLTFGQGTNLEIKRTVAAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC |
| IMP731 | | |
| SEQ ID NO: 764 | Heavy chain | QVQLKESGPGLVAPSQSLSITCTVSGFSLTAYGVNWVRQPPGKG |
| | | LEWLGMIWDDGSTDYNSALKSRLSISKDNSKSQVFLKMNSLQT |
| | | DDTARYYCAREGDVAFDYWGQGTTLTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| SEQ ID NO: 765 | Light chain | DIVMTQSPSSLAVSVGQKVTMSCKSSQSLLNGSNQKNYLAWYQ |
| | | QKPGQSPKLLVYFASTRDSGVPDRFIGSGSGTDFTLTISSVQAED |
| | | LADYFCLQHFGTPPTFGGGTKLEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |

### TIM-3 Inhibitors

In certain embodiments, a combination described herein comprises a TIM-3 inhibitor.

Without wishing to be bound by theory, it is believed that TIM-3 correlates with tumor myeloid signature in The Cancer Genome Atlas (TCGA) database and the most abundant TIM-3 on normal peripheral blood mononuclear cells (PBMCs) is on myeloid cells. TIM-3 is expressed on multiple myeloid subsets in human PBMCs, including, but not limited to, monocytes, macrophages and dendritic cells.

Tumor purity estimates are negatively correlated with TIM-3 expression in a number of TCGA tumor samples (including, e.g., adrenocortical carcinoma (ACC), bladder urothelial carcinoma (BLCA), breast invasive carcinoma (BRCA), cervical squamous cell carcinoma and endocervical adenocarcinoma (CESC), colon adenocarcinoma (COAD), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), brain low grade glioma (LGG), liver hepatocellular carcinoma (LIHC), lung adenocarcinoma (LUAD), lung squamous cell carcinoma (LUSC), ovarian serous cystadenocarcinoma (OV), prostate adenocarcinoma (PRAD), rectum adeiiocarcinoma (READ), skin cutaneous melanoma (SKCM), thyroid carcinoma (THCA), uterine corpus endometrial carcinoma (UCEC), and uterine carcinosarcoma (UCS)), suggesting TIM-3 expression in tumor samples is from tumor infiltrates.

In certain embodiments, the combination is used to treat a kidney cancer (e.g., a kidney renal clear cell carcinoma (KIRC) or a kidney renal papillary cell carcinoma (KIRP)). In other embodiments, the combination is used to treat a brain tumor (e.g., a brain low grade glioma (LGG) or a glioblastoma multiforme (GBM)). In some embodiments, the combination is used to treat a mesothelioma (MESO). In some embodiments, the combination is used to treat a sarcoma (SARC), a lung adenocarcinoma (LUAD), a pancreatic adenocarcinoma (PAAD), or a lung squamous cell carcinoma (LUSC).

Without wishing to be bound by theory, it is believed that in some embodiments, by clustering indications by immune signatures, cancers that can be effectively treated by a combination described herein can be identified, e.g., by determining the fraction of patients in each indication above 75^{th} percentile across TCGA.

In some embodiments, a T cell gene signature comprises expression of one or more (e.g., all) of: CD2, CD247, CD3D, CD3E, CD3G, CD8A, CD8B, CXCR6, GZMK, PYHIN1, SH2D1A, SIRPG or TRAT1.

In some embodiments, a Myeloid gene signature comprises expression of one or more (e.g., all) of SIGLEC1, MSR1, LILRB4, ITGAM or CD163.

In some embodiments, a TIM-3 gene signature comprises expression of one or more (e.g., all) of HAVCR2, ADGRG1, PIK3AP1, CCL3, CCL4, PRF1, CD8A, NKG7, or KLRK1.

Without wishing to be bound by theory, it is believed that in some embodiments, a TIM-3 inhibitor, e.g., MBG453, synergizes with a PD-1 inhibitor, e.g., PDR001, in a mixed lymphocyte reaction (MLR) assay. In some embodiments, inhibition of PD-L1 and TIM-3 results in tumor reduction and survival in mouse models of cancer. In some embodiments, inhibition of PD-L1 and LAG-3 results in tumor reduction and survival in mouse models of cancer.

In some embodiments, the combination is used to treat a cancer having high levels of expression of TIM-3 and one or more of myeloid signature genes (e.g., one or more genes expressed in macrophages). In some embodiments, the cancer having high levels of expression of TIM-3 and myeloid signature genes is chosen from a sarcoma (SARC), a mesothelioma (MESO), a brain tumor (e.g., a glioblastoma (GBM), or a kidney cancer (e.g., a kidney renal papillary cell carcinoma (KIRP)). In other embodiments, the combination is used to treat a cancer having high levels of expression of TIM-3 and one or more of T cell signature genes (e.g., one or more genes expressed in dendritic cells and/or T cells). In some embodiments, the cancer having high levels of expression of TIM-3 and T cell signature genes is chosen from a kidney cancer (e.g., a kidney renal clear cell carcinoma (KIRC)), a lung cancer (e.g., a lung adenocarcinoma (LUAD)), a pancreatic adenocarcinoma (PAAD), or a testicular cancer (e.g., a testicular germ cell tumor (TGCT)).

Without wishing to be bound by theory, it is believed that in some embodiments, by clustering indications by immune signatures, cancers that can be effectively treated by a combination targeting two, three, or more targets described herein can be identified, e.g., by determining the fraction of patients above 75^{th} percentile in both or all of the targets.

In some embodiments, the combination comprises a TIM-3 inhibitor (e.g., a TIM-3 inhibitor described herein) and a PD-1 inhibitor (e.g., a PD-1 inhibitor described herein), e.g., to treat cancer chosen from a kidney cancer (e.g., a kidney renal papillary cell carcinoma (KIRC) or a kidney renal papillary cell carcinoma (KIRP)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a pancreatic cancer (e.g., a pancreatic adenocarcinoma (PAAD)), a cervical cancer (e.g., cervical squamous cell carcinoma and endocervical adenocarcinoma (CESC)), a head and neck cancer (e.g., a head and neck squamous cell carcinoma (HNSC)), a bladder cancer (e.g., bladder urothelial carcinoma (BLCA), a stomach cancer (e.g., stomach adenocarcinoma (STAD)), a skin cancer (e.g., skin cutaneous melanoma (SKCM)), a breast cancer (e.g., breast invasive carcinoma (BRCA)), or a cholangiocarcinoma (CHOL).

In some embodiments, the combination comprises a TIM-3 inhibitor (e.g., a TIM-3 inhibitor described herein) and a LAG-3 inhibitor (e.g., a LAG-3 inhibitor described herein), e.g., to treat cancer chosen from a kidney cancer (e.g., a kidney renal papillary cell carcinoma (KIRC)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a cervical cancer (e.g., cervical squamous cell carcinoma and endocervical adenocarcinoma (CESC)), an ovarian cancer (OV), a head and neck cancer (e.g., a head and neck squamous cell carcinoma (HNSC)), a stomach cancer (e.g., stomach adenocarcinoma (STAD)), a bladder cancer (e.g., bladder urothelial carcinoma (BLCA), a breast cancer (e.g., breast invasive carcinoma (BRCA)), or a skin cancer (e.g., skin cutaneous melanoma (SKCM)).

In some embodiments, the combination comprises a TIM-3 inhibitor (e.g., a TIM-3 inhibitor described herein), a PD-1 inhibitor (e.g., a PD-1 inhibitor described herein), and a LAG-3 inhibitor (e.g., a LAG-3 inhibitor described herein), e.g., to treat a cancer chosen from a kidney cancer (e.g., a kidney renal papillary cell carcinoma (KIRC)), a lung cancer (e.g., a lung adenocarcinoma (LUAD) or a lung squamous cell carcinoma (LUSC)), a mesothelioma (MESO), a testicular cancer (e.g., a testicular germ cell tumor (TGCT)), a sarcoma (SARC), a cervical cancer (e.g., cervical squamous cell carcinoma and endocervical adenocarcinoma (CESC)), a head and neck cancer (e.g., a head and neck squamous cell carcinoma (HNSC)), a stomach cancer (e.g., stomach adenocarcinoma (STAD)), an ovarian cancer (OV), a bladder cancer (e.g., bladder urothelial carcinoma (BLCA), a breast cancer (e.g., breast invasive carcinoma (BRCA)), or a skin cancer (e.g., skin cutaneous melanoma (SKCM)).

In some embodiments, the combination comprises a TIM-3 inhibitor (e.g., a TIM-3 inhibitor described herein), a PD-1 inhibitor (e.g., a PD-1 inhibitor described herein), and a c-MET inhibitor (e.g., a c-MET inhibitor described herein), e.g., to treat a cancer chosen from a kidney cancer (e.g., a kidney renal papillary cell carcinoma (KIRC)), a lung cancer (e.g., a lung adenocarcinoma (LUAD), or a mesothelioma (MESO).

In some embodiments, the TIM-3 inhibitor is MBG453 (Novartis) or TSR-022 (Tesaro). In some embodiments, the TIM-3 inhibitor is MBG453.

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of ABTIM3-hum11 or ABTIM3-hum03 disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 7
Amino acid and nucleotide sequences of exemplary
anti-TIM-3 antibody molecules

| | | |
|---|---|---|
| ABTIM3-hum11 | | |
| SEQ ID NO: 801 (Kabat) | HCDR1 | SYNMH |
| SEQ ID NO: 802 (Kabat) | HCDR2 | DIYPGNGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 805 (Chothia) | HCDR2 | YPGNGD |
| SEQ ID NO: 803 (Chothia) | HCDR3 | VGGAFPMDY |
| SEQ ID NO: 806 | VH | QVQLVQSGAEVKKPGSSVKVSCKASGYTFTSYNMHWV |
| | | RQAPGQGLEWMGDIYPGNGDTSYNQKFKGRVTITADKS |
| | | TSTVYMELSSLRSEDTAVYYCARVGGAFPMDYWGQGTT |
| | | VTVSS |
| SEQ ID NO: 807 | DNA VH | CAGGTGCAGCTGGTGCAGTCAGGCGCCGAAGTGAAGA |
| | | AACCCGGCTCTAGCGTGAAAGTTTCTTGTAAAGCTAGT |
| | | GGCTACACCTTCACTAGCTATAATATGCACTGGGTTCG |
| | | CCAGGCCCCAGGGCAAGGCCTCGAGTGGATGGGCGAT |
| | | ATCTACCCCGGGAACGGCGACACTAGTTATAATCAGA |
| | | AGTTTAAGGGTAGAGTCACTATCACCGCCGATAAGTCT |
| | | ACTAGCACCGTCTATATGGAACTGAGTTCCCTGAGGTC |
| | | TGAGGACACCGCCGTCTACTACTGCGCTAGAGTGGGC |
| | | GGAGCCTTCCCTATGGACTACTGGGGTCAAGGCACTA |
| | | CCGTGACCGTGTCTAGC |
| SEQ ID NO: 808 | Heavy | QVQLVQSGAEVKKPGSSVKVSCKASGYTFTSYNMHWV |
| | chain | RQAPGQGLEWMGDIYPGNGDTSYNQKFKGRVTITADKS |
| | | TSTVYMELSSLRSEDTAVYYCARVGGAFPMDYWGQGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLG |
| SEQ ID NO: 809 | DNA | CAGGTGCAGCTGGTGCAGTCAGGCGCCGAAGTGAAGA |
| | heavy | AACCCGGCTCTAGCGTGAAAGTTTCTTGTAAAGCTAGT |
| | chain | GGCTACACCTTCACTAGCTATAATATGCACTGGGTTCG |
| | | CCAGGCCCCAGGGCAAGGCCTCGAGTGGATGGGCGAT |
| | | ATCTACCCCGGGAACGGCGACACTAGTTATAATCAGA |
| | | AGTTTAAGGGTAGAGTCACTATCACCGCCGATAAGTCT |
| | | ACTAGCACCGTCTATATGGAACTGAGTTCCCTGAGGTC |
| | | TGAGGACACCGCCGTCTACTACTGCGCTAGAGTGGGC |
| | | GGAGCCTTCCCTATGGACTACTGGGGTCAAGGCACTA |
| | | CCGTGACCGTGTCTAGCGCTAGCACTAAGGGCCCGTCC |
| | | GTGTTCCCCCTGGCACCTTGTAGCCGGAGCACTAGCGA |
| | | ATCCACCGCTGCCCTCGGCTGCCTGGTCAAGGATTACT |
| | | TCCCGGAGCCCGTGACCGTGTCCTGGAACAGCGGAGC |
| | | CCTGACCTCCGGAGTGCACACCTTCCCCGCTGTGCTGC |
| | | AGAGCTCCGGGCTGTACTCGCTGTCGTCGGTGGTCACG |
| | | GTGCCTTCATCTAGCCTGGGTACCAAGACCTACACTTG |
| | | CAACGTGGACCACAAGCCTTCCAACACTAAGGTGGAC |
| | | AAGCGCGTCGAATCGAAGTACGGCCCACCGTGCCCGC |
| | | CTTGTCCCGCGCCGGAGTTCCTCGGCGGTCCCTCGGTC |
| | | TTTCTGTTCCCACCGAAGCCCAAGGACACTTTGATGAT |
| | | TTCCCGCACCCCTGAAGTGACATGCGTGGTCGTGGACG |
| | | TGTCACAGGAAGATCCGGAGGTGCAGTTCAATTGGTA |
| | | CGTGGATGGCGTCGAGGTGCACAACGCCAAAACCAAG |
| | | CCGAGGGAGGAGCAGTTCAACTCCACTTACCGCGTCG |
| | | TGTCCGTGCTGACGGTGCTGCATCAGGACTGGCTGAAC |
| | | GGGAAGGAGTACAAGTGCAAAGTGTCCAACAAGGGA |
| | | CTTCCTAGCTCAATCGAAAAGACCATCTCGAAAGCCA |
| | | AGGGACAGCCCCGGGAACCCCAAGTGTATACCCTGCC |
| | | ACCGAGCCAGGAAGAAATGACTAAGAACCAAGTCTCA |
| | | TTGACTTGCCTTGTGAAGGGCTTCTACCCATCGGATAT |
| | | CGCCGTGGAATGGGAGTCCAACGGCCAGCCGGAAAAC |
| | | AACTACAAGACCACCCCTCCGGTGCTGGACTCAGACG |
| | | GATCCTTCTTCCTCTACTCGCGGCTGACCGTGGATAAG |
| | | AGCAGATGGCAGGAGGGAAATGTGTTCAGCTGTTCTG |
| | | TGATGCATGAAGCCCTGCACAACCACTACACTCAGAA |
| | | GTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 816 | VL | AIQLTQSPSSLSASVGDRVTITCRASESVEYYGTSLMQWY |
| | | QQKPGKAPKLLIYAASNVESGVPSRFSGSGSGTDFTLTISS |
| | | LQPEDFATYFCQQSRKDPSTFGGGTKVEIK |
| SEQ ID NO: 817 | DNA VL | GCTATTCAGCTGACTCAGTCACCTAGTAGCCTGAGCGC |
| | | TAGTGTGGGCGATAGAGTGACTATCACCTGTAGAGCT |
| | | AGTGAATCAGTCGAGTACTACGGCACTAGCCTGATGC |
| | | AGTGGTATCAGCAGAAGCCCGGGAAAGCCCCTAAGCT |
| | | GCTGATCTACGCCGCCTCTAACGTGGAATCAGGCGTGC |
| | | CCTCTAGGTTTAGCGGTAGCGGTAGTGGCACCGACTTC |
| | | ACCCTGACTATCTCTAGCCTGCAGCCCGAGGACTTCGC |
| | | TACCTACTTCTGTCAGCAGTCTAGGAAGGACCCTAGCA |
| | | CCTTCGGCGGAGGCACTAAGGTCGAGATTAAG |
| SEQ ID NO: 818 | Light | AIQLTQSPSSLSASVGDRVTITCRASESVEYYGTSLMQWY |
| | chain | QQKPGKAPKLLIYAASNVESGVPSRFSGSGSGTDFTLTISS |
| | | LQPEDFATYFCQQSRKDPSTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| SEQ ID NO: 819 | DNA light | GCTATTCAGCTGACTCAGTCACCTAGTAGCCTGAGCGC |
| | chain | TAGTGTGGGCGATAGAGTGACTATCACCTGTAGAGCT |
| | | AGTGAATCAGTCGAGTACTACGGCACTAGCCTGATGC |
| | | AGTGGTATCAGCAGAAGCCCGGGAAAGCCCCTAAGCT |
| | | GCTGATCTACGCCGCCTCTAACGTGGAATCAGGCGTGC |
| | | CCTCTAGGTTTAGCGGTAGCGGTAGTGGCACCGACTTC |
| | | ACCCTGACTATCTCTAGCCTGCAGCCCGAGGACTTCGC |
| | | TACCTACTTCTGTCAGCAGTCTAGGAAGGACCCTAGCA |
| | | CCTTCGGCGGAGGCACTAAGGTCGAGATTAAGCGTAC |
| | | GGTGGCCGCTCCCAGCGTGTTCATCTTCCCCCCCAGCG |
| | | ACGAGCAGCTGAAGAGCGGCACCGCCAGCGTGGTGTG |
| | | CCTGCTGAACAACTTCTACCCCCGGGAGGCCAAGGTG |
| | | CAGTGGAAGGTGGACAACGCCCTGCAGAGCGGCAACA |
| | | GCCAGGAGAGCGTCACCGAGCAGGACAGCAAGGACTC |
| | | CACCTACAGCCTGAGCAGCACCCTGACCCTGAGCAAG |
| | | GCCGACTACGAGAAGCATAAGGTGTACGCCTGCGAGG |
| | | TGACCCACCAGGGCCTGTCCAGCCCCGTGACCAAGAG |
| | | CTTCAACAGGGGCGAGTGC |
| ABTIM3-hum03 | | |
| SEQ ID NO: 801 (Kabat) | HCDR1 | SYNMH |
| SEQ ID NO: 820 (Kabat) | HCDR2 | DIYPGQGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 821 (Chothia) | HCDR2 | YPGQGD |
| SEQ ID NO: 803 (Chothia) | HCDR3 | VGGAFPMDY |
| SEQ ID NO: 822 | VH | QVQLVQSGAEVKKPGASVKVSCKASGYTFTSYNMHWV |
| | | RQAPGQGLEWIGDIYPGQGDTSYNQKFKGRATMTADKS |
| | | TSTVYMELSSLRSEDTAVYYCARVGGAFPMDYWGQGTL |
| | | VTVSS |
| SEQ ID NO: 823 | DNA VH | CAGGTGCAGCTGGTGCAGTCAGGCGCCGAAGTGAAGA |
| | | AACCCGGCGCTAGTGTGAAAGTTAGCTGTAAAGCTAG |
| | | TGGCTATACTTTCACTTCTTATAATATGCACTGGGTCC |
| | | GCCAGGCCCCAGGTCAAGGCCTCGAGTGGATCGGCGA |
| | | TATCTACCCCGGTCAAGGCGACACTTCCTATAATCAGA |
| | | AGTTTAAGGGTAGAGCTACTATGACCGCCGATAAGTC |
| | | TACTTCTACCGTCTATATGGAACTGAGTTCCCTGAGGT |
| | | CTGAGGACACCGCCGTCTACTACTGCGCTAGAGTGGG |
| | | CGGAGCCTTCCCAATGGACTACTGGGGTCAAGGCACC |
| | | CTGGTCACCGTGTCTAGC |
| SEQ ID NO: 824 | Heavy | QVQLVQSGAEVKKPGASVKVSCKASGYTFTSYNMHWV |
| | chain | RQAPGQGLEWIGDIYPGQGDTSYNQKFKGRATMTADKS |
| | | TSTVYMELSSLRSEDTAVYYCARVGGAFPMDYWGQGTL |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLG |
| SEQ ID NO: 825 | DNA | CAGGTGCAGCTGGTGCAGTCAGGCGCCGAAGTGAAGA |
| | heavy | AACCCGGCGCTAGTGTGAAAGTTAGCTGTAAAGCTAG |
| | chain | TGGCTATACTTTCACTTCTTATAATATGCACTGGGTCC |
| | | GCCAGGCCCCAGGTCAAGGCCTCGAGTGGATCGGCGA |
| | | TATCTACCCCGGTCAAGGCGACACTTCCTATAATCAGA |
| | | AGTTTAAGGGTAGAGCTACTATGACCGCCGATAAGTC |
| | | TACTTCTACCGTCTATATGGAACTGAGTTCCCTGAGGT |
| | | CTGAGGACACCGCCGTCTACTACTGCGCTAGAGTGGG |
| | | CGGAGCCTTCCCAATGGACTACTGGGGTCAAGGCACC |
| | | CTGGTCACCGTGTCTAGCGCTAGCACTAAGGGCCCGTC |
| | | CGTGTTCCCCCTGGCACCTTGTAGCCGGAGCACTAGCG |
| | | AATCCACCGCTGCCCTCGGCTGCCTGGTCAAGGATTAC |
| | | TTCCCGGAGCCCGTGACCGTGTCCTGGAACAGCGGAG |
| | | CCCTGACCTCCGGAGTGCACACCTTCCCCGCTGTGCTG |
| | | CAGAGCTCCGGGCTGTACTCGCTGTCGTCGGTGGTCAC |
| | | GGTGCCTTCATCTAGCCTGGGTACCAAGACCTACACTT |
| | | GCAACGTGGACCACAAGCCTTCCAACACTAAGGTGGA |
| | | CAAGCGCGTCGAATCGAAGTACGGCCCACCGTGCCCG |
| | | CCTTGTCCCGCGCCGGAGTTCCTCGGCGGTCCCTCGGT |
| | | CTTTCTGTTCCCACCGAAGCCCAAGGACACTTTGATGA |
| | | TTTCCCGCACCCCTGAAGTGACATGCGTGGTCGTGGAC |
| | | GTGTCACAGGAAGATCCGGAGGTGCAGTTCAATTGGT |
| | | ACGTGGATGGCGTCGAGGTGCACAACGCCAAAACCAA |
| | | GCCGAGGGAGGAGCAGTTCAACTCCACTTACCGCGTC |
| | | GTGTCCGTGCTGACGGTGCTGCATCAGGACTGGCTGA |
| | | ACGGGAAGGAGTACAAGTGCAAAGTGTCCAACAAGG |
| | | GACTTCCTAGCTCAATCGAAAAGACCATCTCGAAAGC |
| | | CAAGGGACAGCCCCGGGAACCCCAAGTGTATACCCTG |
| | | CCACCGAGCCAGGAAGAAATGACTAAGAACCAAGTCT |
| | | CATTGACTTGCCTTGTGAAGGGCTTCTACCCATCGGAT |
| | | ATCGCCGTGGAATGGGAGTCCAACGGCCAGCCGGAAA |
| | | ACAACTACAAGACCACCCCTCCGGTGCTGGACTCAGA |
| | | CGGATCCTTCTTCCTCTACTCGCGGCTGACCGTGGATA |
| | | AGAGCAGATGGCAGGAGGGAAATGTGTTCAGCTGTTC |
| | | TGTGATGCATGAAGCCCTGCACAACCACTACACTCAG |
| | | AAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 826 | VL | DIVLTQSPDSLAVSLGERATINCRASESVEYYGTSLMQW |
| | | YQQKPGQPPKLLIYAASNVESGVPDRFSGSGSGTDFTLTI |
| | | SSLQAEDVAVYYCQQSRKDPSTFGGGTKVEIK |
| SEQ ID NO: 827 | DNA VL | GATATCGTCCTGACTCAGTCACCCGATAGCCTGGCCGT |
| | | CAGCCTGGGCGAGCGGGCTACTATTAACTGTAGAGCT |
| | | AGTGAATCAGTCGAGTACTACGGCACTAGCCTGATGC |
| | | AGTGGTATCAGCAGAAGCCCGGTCAACCCCCTAAGCT |
| | | GCTGATCTACGCCGCCTCTAACGTGGAATCAGGCGTGC |
| | | CCGATAGGTTTAGCGGTAGCGGTAGTGGCACCGACTT |
| | | CACCCTGACTATTAGTAGCCTGCAGGCCGAGGACGTG |
| | | GCCGTCTACTACTGTCAGCAGTCTAGGAAGGACCCTA |
| | | GCACCTTCGGCGGAGGCACTAAGGTCGAGATTAAG |
| SEQ ID NO: 828 | Light | DIVLTQSPDSLAVSLGERATINCRASESVEYYGTSLMQW |
| | chain | YQQKPGQPPKLLIYAASNVESGVPDRFSGSGSGTDFTLTI |
| | | SSLQAEDVAVYYCQQSRKDPSTFGGGTKVEIKRTVAAPS |
| | | VFIFPPSDEQLKSGTASVVCLLNNFYPREAKVQWKVDNA |
| | | LQSGNSQESVTEQDSKDSTYSLSSTLTLSKADYEKHKVY |
| | | ACEVTHQGLSSPVTKSFNRGEC |
| SEQ ID NO: 829 | DNA light | GATATCGTCCTGACTCAGTCACCCGATAGCCTGGCCGT |
| | chain | CAGCCTGGGCGAGCGGGCTACTATTAACTGTAGAGCT |
| | | AGTGAATCAGTCGAGTACTACGGCACTAGCCTGATGC |
| | | AGTGGTATCAGCAGAAGCCCGGTCAACCCCCTAAGCT |
| | | GCTGATCTACGCCGCCTCTAACGTGGAATCAGGCGTGC |
| | | CCGATAGGTTTAGCGGTAGCGGTAGTGGCACCGACTT |
| | | CACCCTGACTATTAGTAGCCTGCAGGCCGAGGACGTG |
| | | GCCGTCTACTACTGTCAGCAGTCTAGGAAGGACCCTA |
| | | GCACCTTCGGCGGAGGCACTAAGGTCGAGATTAAGCG |
| | | TACGGTGGCCGCTCCCAGCGTGTTCATCTTCCCCCCCA |
| | | GCGACGAGCAGCTGAAGAGCGGCACCGCCAGCGTGGT |
| | | GTGCCTGCTGAACAACTTCTACCCCCGGGAGGCCAAG |
| | | GTGCAGTGGAAGGTGGACAACGCCCTGCAGAGCGGCA |
| | | ACAGCCAGGAGAGCGTCACCGAGCAGGACAGCAAGG |
| | | ACTCCACCTACAGCCTGAGCAGCACCCTGACCCTGAG |
| | | CAAGGCCGACTACGAGAAGCATAAGGTGTACGCCTGC |
| | | GAGGTGACCCACCAGGGCCTGTCCAGCCCCGTGACCA |
| | | AGAGCTTCAACAGGGGCGAGTGC |

In some embodiments, the TIM-3 inhibitor is administered at a dose of about 50 mg to about 100 mg, about 200 mg to about 250 mg, about 500 mg to about 1000 mg, or about 1000 mg to about 1500 mg. In embodiments, the TIM-3 inhibitor is administered once every 4 weeks. In other embodiments, the TIM-3 inhibitor is administered at a dose of about 50 mg to about 100 mg once every four weeks. In other embodiments, the TIM-3 inhibitor is administered at a dose of about 200 mg to about 250 mg once every four weeks. In other embodiments, the TIM-3 inhibitor is administered at a dose of about 500 mg to about 1000 mg once every four weeks. In other embodiments, the TIM-3 inhibitor is administered at a dose of about 1000 mg to about 1500 mg once every four weeks.

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 8. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 8
Amino acid sequences of other exemplary anti-TIM-3 antibody molecules

| | | |
|---|---|---|
| APE5137 | | |
| SEQ ID NO: 830 | VH | EVQLLESGGGLVQPGGSLRLSCAAASGFTFSSYDMSWVRQAPGKGL |
| | | DWVSTISGGGTYTYYQDSVKGRFTISRDNSKNTLYLQMNSLRAEDTA |
| | | VYYCASMDYWGQGTTVTVSSA |
| SEQ ID NO: 831 | VL | DIQMTQSPSSLSASVGDRVTITCRASQSIRRYLNWYHQKPGKAPKLLI |
| | | YGASTLQSGVPSRFSGSGSGTDFTLTISSLQPEDFAVYYCQQSHSAPLT |
| | | FGGGTKVEIKR |
| APE5121 | | |
| SEQ ID NO: 832 | VH | EVQVLESGGGLVQPGGSLRLYCVASGFTFSGSYAMSWVRQAPGKGL |
| | | EWVSAISGSGGSTYYADSVKGRFTISRDNSKNTLYLQMNSLRAEDTA |
| | | VYYCAKKYYVGPADYWGQGTLVTVSSG |
| SEQ ID NO: 833 | VL | DIVMTQSPDSLAVSLGERATINCKSSQSVLYSSNNKNYLAWYQHKPG |
| | | QPPKLLIYWASTRESGVPDRFSGSGSGTDFTLTISSLQAEDVAVYYCQ |
| | | QYYSSPLTFGGGTKIEVK |

### GITR Agonists

Glucocorticoid-induced TNFR-related protein (GITR) is a member of the Tumor Necrosis Factor Superfamily (TNFRSF). GITR expression is detected constitutively on murine and human CD4+CD25+ regulatory T cells which can be further increased upon activation. In contrast, effector CD4+CD25− T cells and CD8+CD25− T cells express low to undetectable levels of GITR, which is rapidly upregulated following T cell receptor activation. Expression of GITR has also been detected on activated NK cells, dendritic cells, and macrophages. Signal transduction pathway downstream of GITR has been shown to involve MAPK and the canonical NFκB pathways. Various TRAF family members have been implicated as signaling intermediates downstream of GITR (Nocentini et al. (2005) Eur. J Immunol. 35:1016-1022).

Cellular activation through GITR is believed to serve several functions depending on the cell type and microenvironment including, but not limited to, costimulation to augment proliferation and effector function, inhibition of suppression by regulatory T cells, and protection from activation-induced cell death (Shevach and Stephens (2006) Nat. Rev. Immunol. 6:613-618). An agonistic monoclonal antibody against mouse GITR effectively induced tumor-specific immunity and eradicated established tumors in a mouse syngeneic tumor model (Ko et al. (2005) J. Exp. Med. 202:885-891).

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of MAB7 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 9.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 9
Amino acid and nucleotide sequences of exemplary anti-GITR antibody molecule

| | | |
|---|---|---|
| MAB7 | | |
| SEQ ID NO: 901 | VH | EVQLVESGGGLVQSGGSLRLSCAASGFSLSSYGVDWVRQ |
| | | APGKGLEWVGVIWGGGGTYYASSLMGRFTISRDNSKNTL |
| | | YLQMNSLRAEDTAVYYCARHAYGHDGGFAMDYWGQGT |
| | | LVTVSS |
| SEQ ID NO: 902 | VL | EIVMTQSPATLSVSPGERATLSCRASESVSSNVAWYQQRP |
| | | GQAPRLLIYGASNRATGIPARFSGSGSGTDFTLTISRLEPED |
| | | FAVYYCGQSYSYPFTFGQGTKLEIK |
| SEQ ID NO: 903 | Heavy | EVOLVESGGGLVQSGGSLRLSCAASGFSLSSYGVDWVRQ |
| | Chain | APGKGLEWVGVIWGGGGTYYASSLMGRFTISRDNSKNTL |
| | | YLQMNSLRAEDTAVYYCARHAYGHDGGFAMDYWGQGT |
| | | LVTVSSASTKGPSVFPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKRVEPKSCDKTHTCPPCPAPE |
| | | LLGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPAPIEKTISKAKGQPREPQVYTLP |
| | | PSREEMTKNQVSLTCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| SEQ ID NO: 904 | Light | EIVMTQSPATLSVSPGERATLSCRASESVSSNVAWYQQRP |
| | Chain | GQAPRLLIYGASNRATGIPARFSGSGSGTDFTLTISRLEPED |
| | | FAVYYCGQSYSYPFTFGQGTKLEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 905 | DNA VH | GAGGTGCAGCTGGTGGAATCTGGCGGCGGACTGGTGCA |
| | | GTCCGGCGGCTCTCTGAGACTGTCTTGCGCTGCCTCCGG |
| | | CTTCTCCCTGTCCTCTTACGGCGTGGACTGGGTGCGACA |
| | | GGCCCCTGGCAAGGGCCTGGAATGGGTGGGAGTGATCT |
| | | GGGGCGGAGGCGGCACCTACTACGCCTCTTCCCTGATG |
| | | GGCCGGTTCACCATCTCCCGGGACAACTCCAAGAACAC |
| | | CCTGTACCTGCAGATGAACTCCCTGCGGGCCGAGGACA |
| | | CCGCCGTGTACTACTGCGCCAGACACGCCTACGGCCAC |
| | | GACGGCGGCTTCGCCATGGATTATTGGGGCCAGGGCAC |
| | | CCTGGTGACAGTGTCCTCC |
| SEQ ID NO: 906 | DNA VL | GAGATCGTGATGACCCAGTCCCCCGCCACCCTGTCTGT |
| | | GTCTCCCGGCGAGAGAGCCACCCTGAGCTGCAGAGCCT |
| | | CCGAGTCCGTGTCCTCCAACGTGGCCTGGTATCAGCAG |
| | | AGACCTGGTCAGGCCCCTCGGCTGCTGATCTACGGCGC |
| | | CTCTAACCGGGCCACCGGCATCCCTGCCAGATTCTCCG |
| | | GCTCCGGCAGCGGCACCGACTTCACCCTGACCATCTCC |
| | | CGGCTGGAACCCGAGGACTTCGCCGTGTACTACTGCGG |
| | | CCAGTCCTACTCATACCCCTTCACCTTCGGCCAGGGCAC |
| | | CAAGCTGGAAATCAAG |
| SEQ ID NO: 907 | DNA | GAGGTGCAGCTGGTGGAATCTGGCGGCGGACTGGTGCA |
| | Heavy | GTCCGGCGGCTCTCTGAGACTGTCTTGCGCTGCCTCCGG |
| | Chain | CTTCTCCCTGTCCTCTTACGGCGTGGACTGGGTGCGACA |
| | | GGCCCCTGGCAAGGGCCTGGAATGGGTGGGAGTGATCT |
| | | GGGGCGGAGGCGGCACCTACTACGCCTCTTCCCTGATG |
| | | GGCCGGTTCACCATCTCCCGGGACAACTCCAAGAACAC |
| | | CCTGTACCTGCAGATGAACTCCCTGCGGGCCGAGGACA |
| | | CCGCCGTGTACTACTGCGCCAGACACGCCTACGGCCAC |
| | | GACGGCGGCTTCGCCATGGATTATTGGGGCCAGGGCAC |
| | | CCTGGTGACAGTGTCCTCCGCTAGCACCAAGGGCCCAA |
| | | GTGTGTTTCCCCTGGCCCCCAGCAGCAAGTCTACTTCCG |
| | | GCGGAACTGCTGCCCTGGGTTGCCTGGTGAAGGACTAC |
| | | TTCCCCGAGCCCGTGACAGTGTCCTGGAACTCTGGGGC |
| | | TCTGACTTCCGGCGTGCACACCTTCCCCGCCGTGCTGCA |
| | | GAGCAGCGGCCTGTACAGCCTGAGCAGCGTGGTGACAG |
| | | TGCCCTCCAGCTCTCTGGGAACCCAGACCTATATCTGCA |
| | | ACGTGAACCACAAGCCCAGCAACACCAAGGTGGACAA |
| | | GAGAGTGGAGCCCAAGAGCTGCGACAAGACCCACACC |
| | | TGCCCCCCCTGCCCAGCTCCAGAACTGCTGGGAGGGCC |
| | | TTCCGTGTTCCTGTTCCCCCCCAAGCCCAAGGACACCCT |
| | | GATGATCAGCAGGACCCCCGAGGTGACCTGCGTGGTGG |
| | | TGGACGTGTCCCACGAGGACCCAGAGGTGAAGTTCAAC |
| | | TGGTACGTGGACGGCGTGGAGGTGCACAACGCCAAGA |
| | | CCAAGCCCAGAGAGGAGCAGTACAACAGCACCTACAG |
| | | GGTGGTGTCCGTGCTGACCGTGCTGCACCAGGACTGGC |
| | | TGAACGGCAAAGAATACAAGTGCAAAGTCTCCAACAA |
| | | GGCCCTGCCAGCCCCAATCGAAAAGACAATCAGCAAG |
| | | GCCAAGGGCCAGCCACGGGAGCCCCAGGTGTACACCCT |
| | | GCCCCCCAGCCGGGAGGAGATGACCAAGAACCAGGTG |
| | | TCCCTGACCTGTCTGGTGAAGGGCTTCTACCCCAGCGAT |
| | | ATCGCCGTGGAGTGGGAGAGCAACGGCCAGCCCGAGA |
| | | ACAACTACAAGACCACCCCCCCAGTGCTGGACAGCGAC |
| | | GGCAGCTTCTTCCTGTACAGCAAGCTGACCGTGGACAA |
| | | GTCCAGGTGGCAGCAGGGCAACGTGTTCAGCTGCAGCG |
| | | TGATGCACGAGGCCCTGCACAACCACTACACCCAGAAG |
| | | TCCCTGAGCCTGAGCCCCGGCAAG |
| SEQ ID NO: 908 | DNA | GAGATCGTGATGACCCAGTCCCCCGCCACCCTGTCTGT |
| | Light | GTCTCCCGGCGAGAGAGCCACCCTGAGCTGCAGAGCCT |
| | Chain | CCGAGTCCGTGTCCTCCAACGTGGCCTGGTATCAGCAG |
| | | AGACCTGGTCAGGCCCCTCGGCTGCTGATCTACGGCGC |
| | | CTCTAACCGGGCCACCGGCATCCCTGCCAGATTCTCCG |
| | | GCTCCGGCAGCGGCACCGACTTCACCCTGACCATCTCC |
| | | CGGCTGGAACCCGAGGACTTCGCCGTGTACTACTGCGG |
| | | CCAGTCCTACTCATACCCCTTCACCTTCGGCCAGGGCAC |
| | | CAAGCTGGAAATCAAGCGTACGGTGGCCGCTCCCAGCG |
| | | TGTTCATCTTCCCCCCCAGCGACGAGCAGCTGAAGAGC |
| | | GGCACCGCCAGCGTGGTGTGCCTGCTGAACAACTTCTA |
| | | CCCCCGGGAGGCCAAGGTGCAGTGGAAGGTGGACAAC |
| | | GCCCTGCAGAGCGGCAACAGCCAGGAGAGCGTCACCG |
| | | AGCAGGACAGCAAGGACTCCACCTACAGCCTGAGCAG |
| | | CACCCTGACCCTGAGCAAGGCCGACTACGAGAAGCATA |
| | | AGGTGTACGCCTGCGAGGTGACCCACCAGGGCCTGTCC |
| | | AGCCCCGTGACCAAGAGCTTCAACAGGGGCGAGTGC |
| SEQ ID NO: 909 (KABAT) | HCDR1 | SYGVD |
| SEQ ID NO: 910 (CHOTHIA) | HCDR1 | GFSLSSY |
| SEQ ID NO: 911 (KABAT) | HCDR2 | VIWGGGGTYYASSLMG |
| SEQ ID NO: 912 (CHOTHIA) | HCDR2 | WGGGG |
| SEQ ID NO: 913 (KABAT) | HCDR3 | HAYGHDGGFAMDY |
| SEQ ID NO: 913 (CHOTHIA) | HCDR3 | HAYGHDGGFAMDY |
| SEQ ID NO: 914 (KABAT) | LCDR1 | RASESVSSNVA |
| SEQ ID NO: 915 (CHOTHIA) | LCDR1 | SESVSSN |
| SEQ ID NO: 916 (KABAT) | LCDR2 | GASNRAT |
| SEQ ID NO: 917 (CHOTHIA) | LCDR2 | GAS |
| SEQ ID NO: 918 (KABAT) | LCDR3 | GQSYSYPFT |
| SEQ ID NO: 919 (CHOTHIA) | LCDR3 | SYSYPF |

In some embodiments, the GITR agonist is administered at a dose of about 2 mg to about 600 mg (e.g., about 5 mg to about 500 mg). In some embodiments, the GITR agonist is administered once every week. In other embodiments, the GITR agonist is administered once every three weeks. In other embodiments, the GITR agonist is administered once every six weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every week.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every three weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every six weeks.

In some embodiments, three doses of the GITR agonist are administered over a period of three weeks followed by a nine-week pause. In some embodiments, four doses of the GITR agonist are administered over a period of twelve weeks followed by a nine-week pause. In some embodiments, four doses of the GITR agonists are administered over a period of twenty-one or twenty-four weeks followed by a nine-week pause.

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 10.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et a. Cancer Res 2016; 76 (14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

In one embodiment, the anti-GITR antibody molecule is an anti-GITR antibody molecule disclosed in WO 2013/039954, herein incorporated by reference in its entirety. In an embodiment, the anti-GITR antibody molecule is an anti-GITR antibody molecule disclosed in US 2014/0072566, herein incorporated by reference in its entirety.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 10
Amino acid sequence of other exemplary
anti-GITR antibody molecules

| | | |
|---|---|---|
| BMS-986156 | | |
| SEQ ID | VH | QVQLVESGGGVVQPGRSLRLSCAASGFTFSSY |
| MO: 920 | | GMHWVRQAPGKGLEWVAVIWYEGSNKYYADSV |
| | | KGRFTISRDNSKNTLYLQMNSLRAEDTAVYYC |
| | | ARGGSMVRGDYYYGMDVWGQGTTVTVSS |
| SEQ ID | VL | AIQLTQSPSSLSASVGDRVTITCRASQGISSA |
| NO: 921 | | LAWYQQKPGKAPKLLIYDASSLESGVPSRFSG |
| | | SGSGTDFTLTISSLQPEDFATYYCQQFNSYPY |
| | | TFGQGTKLEIK |

### TGF-β Inhibitors

In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signaling pathways are involved in cellular responses to TGF-β. Activation of the TGF-β/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of TGF-β signaling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination comprising a TGF-β inhibitor described herein is used to treat a cancer in a late stage, a metastatic cancer, or an advanced cancer.

Preclinical evidence indicates that TGF-β plays an important role in immune regulation (Wojtowicz-Praga-Invest New Drugs. 2003; 21(1):21-32; Yang et al. Trends Immunol. 2010; 31(6):220-7). TGF-β can down-regulate the host-immune response via several mechanisms, e.g., shift of the T-helper balance toward Th2 immune phenotype; inhibition of anti-tumoral Th1 type response and M1-type macrophages; suppression of cytotoxic CD8+T lymphocytes (CTL), NK lymphocytes and dendritic cell functions, generation of CD4+CD25+T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7 (5-6):423-35; Achyut et al Gastroenterology. 2011; 141(4):1167-78).

In some embodiments, the TGF-β inhibitor is used in combination with a PD-1 inhibitor, and one or more (e.g., two, three, four, or all) of LAG-3 inhibitor, a GITR agonist, a c-MET inhibitor, an IDO inhibitor, or an A2aR antagonist. In some embodiments, the combination is used to treat a pancreatic cancer, a colorectal cancer, a gastric cancer, or a melanoma (e.g., a refractory melanoma). In some embodiments, the TGF-β inhibitor is chosen from fresolimumab or XOMA 089.

### Exemplary TGF-βInhibitors

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has
the amino acid sequence of:
(SEQ ID NO: 240)
QVQLVQSGAEVKKPGSSVKVSCKASGGTFSSYAISWVRQAPGQGLEWMG
GIIPIFGTANYAQKFQGRVTITADESTSTAYMELSSLRSEDTAVYYCAR
GLWEVRALPSVYWGQGTLVTVSS
(disclosed as SEQ ID NO: 6 in WO 2012/167143).
The light chain variable region of XOMA 089 has
the amino acid sequence of:
(SEQ ID NO: 241)
SYELTQPPSVSVAPGQTARITCGANDIGSKSVHWYQQKAGQAPVLVVSE
DIIRPSGIPERISGSNSGNTATLTISRVEAGDEADYYCQVWDRDSDQYV
FGTGTKVTVLG
(disclosed as SEQ ID NO: 8 in WO 2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

Without wishing to be bound by theory, it is believed that in some embodiments, resistance to PD-1 immunotherapy is associated with the presence of a transcriptional signature which includes, e.g., genes connected to TGF-βsignaling and TGF-β-dependent processes, e.g., wound healing or angiogenesis (Hugo et al. Cell. 2016; 165(1):35-44). In some embodiments, TGF-βblockade extends the therapeutic window of a therapy that inhibits the PD-1/PD-L1 axis. TGF-β inhibitors can affect the clinical benefits of PD-1 immunotherapy, e.g., by modulating tumor microenvironment, e.g., vasculogenesis, fibrosis, or factors that affect the recruitment of effector T cells (Yang et al. Trends Immunol. 2010; 31(6):220-7; Wakefield and Hill Nat Rev Cancer. 2013; 13(5):328-41; Truty and Urrutia Pancreatology. 2007; 7(5-6):423-35).

Without wishing to be bound by theory, it is also believed that in some embodiments, a number of elements of the anti-tumor immunity cycle express both PD-1 and TGF-β receptors, and PD-1 and TGF-β receptors are likely to propagate non-redundant cellular signals. For example, in mouse models of autochthonous prostate cancer, the use of either a dominant-negative form of TGFBRII, or abrogation of TGF-β production in T cells delays tumor growth (Donkor et al. Immunity. 2011; 35(1):123-34; Diener et al. Lab Invest. 2009; 89(2):142-51). Studies in the transgenic adenocarcinoma of the mouse prostate (TRAMP) mice showed that blocking TGF-β signaling in adoptively transferred T cells increases their persistence and antitumor activity (Chou et al. J Immunol. 2012; 189(8):3936-46). The antitumor activity of the transferred T cells may decrease overtime, partially due to PD-1 upregulation in tumor-infiltrating lymphocytes, supporting a combination of PD-1 and TGF-β inhibition as described herein. The use of neutralizing antibodies against either PD-1 or TGF-β can also affect Tregs, given their high expression levels of PD-1 and their responsiveness to TGF-β stimulation (Riella et al. Am J Transplant. 2012; 12(10):2575-87), supporting a combination of PD-1 and TGF-β inhibition to treat cancer, e.g., by enhancing the modulation of Tregs differentiation and function.

Without wishing to be bound by theory, it is believed that cancers can use TGF-β to escape immune surveillance to facilitate tumor growth and metastatic progression. For example, in certain advanced cancers, high levels of TGF-β are associated with tumor aggressiveness and poor prognosis, and TGF-β pathway can promote one or more of cancer cell motility, invasion, EMT, or a stem cell phenotype. Immune regulation mediated by cancer cells and leukocyte populations (e.g., through a variety of cell-expressed or secreted molecules, e.g., IL-10 or TGF-β) may limit the response to checkpoint inhibitors as monotherapy in certain patients. In certain embodiments, a combined inhibition of TGF-β with a checkpoint inhibitor (e.g., an inhibitor of PD-1 described herein) is used to treat a cancer that does not respond, or responds poorly, to a checkpoint inhibitor (e.g., anti-PD-1) monotherapy, e.g., a pancreatic cancer or a colorectal cancer (e.g., a microsatellite stable colorectal cancer (MSS-CRC)). In other embodiments, a combined inhibition of TGF-β with a checkpoint inhibitor (e.g., an inhibitor of PD-1 described herein) is used to treat a cancer that shows a high level of effector T cell infiltration, e.g., a lung cancer (e.g., a non-small cell lung cancer), a breast cancer (e.g., a triple negative breast cancer), a liver cancer (e.g., a hepatocellular carcinoma), a prostate cancer, or a renal cancer (e.g., a clear cell renal cell carcinoma). In some embodiments, the combination of a TGF-β inhibitor and an inhibitor of PD-1 results in a synergistic effect.

In one embodiment, the TGF-β inhibitor (e.g., XOMA 089) is administered at a dose between 0.1 mg/kg and 20 mg/kg, e.g., between 0.1 mg/kg and 15 mg/kg, between 0.1 mg/kg and 12 mg/kg, between 0.3 mg/kg and 6 mg/kg, between 1 mg/kg and 3 mg/kg, between 0.1 mg/kg and 1 mg/kg, between 0.1 mg/kg and 0.5 mg/kg, between 0.1 mg/kg and 0.3 mg/kg, between 0.3 mg/kg and 3 mg/kg, between 0.3 mg/kg and 1 mg/kg, between 3 mg/kg and 6 mg/kg, or between 6 mg/kg and 12 mg/kg, e.g., at a dose of about 0.1 mg/kg, 0.3 mg/kg, 0.5 mg/kg, 1 mg/kg, 3 mg/kg, 6 mg/kg, 12 mg/kg, or 15 mg/kg, e.g., once every week, once every two weeks, once every three weeks, once every four weeks, or once every six weeks.

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks. For example, the TGF-β inhibitor (e.g., XOMA 089) can be administered at a dose between 0.1 mg/kg and 1 mg/kg (e.g., between 0.1 mg/kg and 1 mg/kg, e.g., 0.3 mg/kg), e.g., once every three weeks. In one embodiment, the TGF-β inhibitor (e.g., XOMA 089) is administered intravenously.

In some embodiments, the TGF-β inhibitor is administered in combination with a PD-1 inhibitor (e.g., an anti-PD-1 antibody molecule).

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks, e.g., intravenously, and the PD-1 inhibitor (e.g., the anti-PD-1 antibody molecule) is administered at a dose between 50 mg and 500 mg (e.g., between 100 mg and 400 mg, e.g., at a dose of about 100 mg, 200 mg, 300 mg, or 400 mg), e.g., once every 3 weeks or once every 4 weeks, e.g., by intravenous infusion. In some embodiments, the PD-1 inhibitor (e.g., the anti-PD-1 antibody molecule) is administered at a dose between 100 mg and 300 mg (e.g., at a dose of about 100 mg, 200 mg, or 300 mg), e.g., once every 3 weeks, e.g., by intravenous infusion.

In some embodiments, the TGF-β inhibitor (e.g., XOMA 089) is administered at a dose of about 0.1 mg/kg or 0.3 mg/kg, e.g., once every 3 weeks, e.g., by intravenous infusion, and the PD-1 inhibitor (e.g., the anti-PD-1 antibody molecule) is administered at a dose of about 100 mg, e.g., once every 3 weeks, e.g., by intravenous infusion. In some embodiments, the TGF-β inhibitor (e.g., XOMA 089) is administered at a dose of about 0.3 mg/kg, e.g., once every 3 weeks, e.g., by intravenous infusion, and the PD-1 inhibitor (e.g., the anti-PD-1 antibody molecule) is administered at a dose of about 100 mg or 300 mg, e.g., once every 3 weeks, e.g., by intravenous infusion. In some embodiments, the TGF-β inhibitor (e.g., XOMA 089) is administered at a dose of about 1 mg/kg, 3 mg/kg, 6 mg/kg, 12 mg/kg, or 15 mg/kg, e.g., once every 3 weeks, e.g., by intravenous infusion, and the PD-1 inhibitor (e.g., the anti-PD-1 antibody molecule) is administered at a dose of about 300 mg, e.g., once every 3 weeks, e.g., by intravenous infusion.

In some embodiments, the TGF-β inhibitor (e.g., XOMA 089) is administered at a dose between 0.1 mg and 0.2 mg (e.g., about 0.1 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 0.5 mg and 2 mg (e.g., about 1 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 2 mg and 5 mg (e.g., about 3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 5 mg and 10 mg (e.g., about 6 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 15 mg (e.g., about 12 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 20 mg (e.g., about 15 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered before the PD-1 inhibitor (e.g., the anti-PD-1 antibody molecule) is administered. In other embodiments, the TGF-β inhibitor (e.g., XOMA 089) is administered after the PD-1 inhibitor (e.g., the anti-PD-1 antibody molecule) is administered. In certain embodiments, the TGF-β inhibitor (e.g., XOMA 089) and the PD-1 inhibitor (e.g., the anti-PD-1 antibody molecule), are administered separately with at least a 30-minute (e.g., at least 1, 1.5, or 2 hours) break between the two administrations.

In some embodiments, the combination comprises a PD-1 inhibitor (e.g., a PD-1 inhibitor described herein), a TGF-β inhibitor (e.g., a TGF-β inhibitor described herein) and one or more of a MEK inhibitor (e.g., a MEK inhibitor described herein), an IL-10 inhibitor (e.g., a IL-1b inhibitor described herein) or an A2aR antagonist (e.g., an A2aR antagonist described herein). Without wishing to be bound by theory, it is believe that in some embodiments TGFβ facilitates immunosuppression by Treg subsets in CRC and pancreatic cancer. In some embodiments, the combination comprising a PD-1 inhibitor, a TGF-β inhibitor, and one or more of a MEK inhibitor, an IL-1b inhibitor or an A2aR antagonist is administered in a therapeutically effective amount to a subject, e.g., with CRC or pancreatic cancer.

In some embodiments, a combination comprising a PD-1 inhibitor (e.g., a PD-1 inhibitor described herein), and a TGF-β inhibitor (e.g., a TGF-β inhibitor described herein) shows improved efficacy in controlling tumor growth in a murine MC38 CRC model compared to either single agent alone. Without wishing to be bound by theory, it is believed that in some embodiments a TGF-β inhibitor in combination with a PD-1 inhibitor improves, e.g., increases, the efficacy of the PD-1 inhibitor. In some embodiments, a combination comprising a PD-1 inhibitor (e.g., a PD-1 inhibitor described herein), and a TGF-β inhibitor (e.g., a TGF-β inhibitor described herein) administered to a subject with, e.g., a CRC, can result in an improved, e.g., increased, efficacy of the PD-1 inhibitor.

### Other Exemplary TGF-βInhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino
acid sequence of:
(SEQ ID NO: 238)
QVQLVQSGAEVKKPGSSVKVSCKASGYTFSSNVISWVRQAPGQGLEWMG
GVIPIVDIANYAQRFKGRVTITADESTSTTYMELSSLRSEDTAVYYCAS
TLGLVLDAMDYWGQGTLVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK.
The light chain of fresolimumab has the amino
acid sequence of:
(SEQ ID NO: 239)
ETVLTQSPGTLSLSPGERATLSCRASQSLGSSYLAWYQQKPGQAPRLLI
YGASSRAPGIPDRFSGSGSGTDFTLTISRLEPEDFAVYYCQQYADSPIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### IL-15/IL-15Ra Complexes

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. Without wishing to be bound by theory, it is believed that in some embodiments, IL-15 potentiates, e.g., enhances, Natural Killer cells to eliminate, e.g., kill, pancreatic cancer cells. In an embodiment, response, e.g., therapeutic response, to a combination described herein, e.g., a combination comprising an IL-15/IL15Ra complex, in, e.g., an animal model of colorectal cancer is associated with Natural Killer cell infiltration.

### Exemplary IL-15 IL-15Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 11 and the soluble form of human IL-15Ra comprises an amino acid sequence of SEQ ID NO: 1002 in Table 11, as described in WO 2014/066527, incorporated by reference in its entirety. The molecules described herein can be made by vectors, host cells, and methods described in WO 2007/084342, incorporated by reference in its entirety.

TABLE 11
Amino acid and nucleotide sequences exemplary
IL-15/IL-15Ra complexes
NIZ985

| | | |
|---|---|---|
| SEQ ID NO: | Human IL-15 | NWVNVISDLKKIEDLIQSMHIDATLYTE |
| 1001 | | SDVHPSCKVTAMKCFLLELQVISLESGD |
| | | ASIHDTVENLIILANNSLSSNGNVTESG |
| | | CKECEELEEKNIKEFLQSFVHIVQMFIN |
| | | TS |
| SEQ ID NO: | Human | ITCPPPMSVEHADIWVKSYSLYSRERYI |
| 1002 | Soluble IL- | CNSGFKRKAGTSSLTECVLNKATNVAHW |
| | 15Ra | TTPSLKCIRDPALVHQRPAPPSTVTTAG |
| | | VTPQPESLSPSGKEPAASSPSSNNTAAT |
| | | TAAIVPGSQLMPSKSPSTGTTEISSHES |
| | | SHGTPSQTTAKNWELTASASHQPPGVYP |
| | | QG |

Without wishing to be bound by theory, it is believed that in microsatellite stable CRCs with low T cell infiltration, IL-15 may promote, e.g., increase, T cell priming (e.g., as described in Lou, K. J. SciBX 7(16); 10.1038/SCIBX.2014.449). In some embodiments, the combination comprises a PD-1 inhibitor (e.g., a PD-1 inhibitor described herein), an IL-15/IL15RA complex (e.g., an IL-15/IL15RA complex described herein) and one or more of a MEK inhibitor (e.g., a MEK inhibitor described herein), an IL-1b inhibitor (e.g., a IL-1b inhibitor described herein) or an A2aR antagonist (e.g., an A2aR antagonist described herein). In some embodiments, the combination promotes, e.g., increases T cell priming. Without wishing to be bound by theory, it is further believed that IL-15 may induce NK cell infiltration. In some embodiments, response to a PD-1 inhibitor, an IL-15/IL-15RA complex and one or more of a MEK inhibitor, an IL-1b inhibitor, or an A2Ar antagonist can result in NK cell infiltration.

### Other Exemplary IL-15 IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 12.

TABLE 12
Amino acid sequences of other exemplary IL-15/IL-15Ra complexes
ALT-803 (Altor)

| | | |
|---|---|---|
| SEQ ID NO: 1003 | IL-15N72D | NWVNVISDLKKIEDLIQSMHIDATLYTESDVHPSCKVTAM |
| 1003 | | KCFLLELQVISLESGDASIHDTVENLIILANDSLSSNGNVTE |
| | | SGCKECEELEEKNIKEFLQSFVHIVQMFINTS |
| SEQ ID NO: 1004 | IL-15RaSu/Fc | ITCPPPMSVEHADIWVKSYSLYSRERYICNSGFKRKAGTSS |
| | | LTECVLNKATNVAHWTTPSLKCIREPKSCDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |

IL-15/IL-15Ra sushi domain fusion (Cytune)

| | | |
|---|---|---|
| SEQ ID NO: 1005 | Human IL-15 | NWVNVISDLKKIEDLIQSMHIDATLYTESDVHPSCKVTAM |
| | | KCFLLELQVISLESGDASIHDTVENLIILANNSLSSNGNVTE |
| | | SGCKECEELEXKNIKEFLQSFVHIVQMFINTS |
| | | Where X is E or K |
| SEQ ID NO: 1006 | Human IL- | ITCPPPMSVEHADIWVKSYSLYSRERYICNSGFKRKAGTSS |
| | 15Ra sushi | LTECVLNKATNVAHWTTPSLKCIRDPALVHQRPAPP |
| | and hinge | |
| | domains | |

### PRRT Agents

For the present invention, the PRRT agents are complexes formed by the radionuclide 177Lu and the cell receptor binding moiety linked to a chelating agent.
The cell receptor binding moiety and the chelating agent may form together the following molecules:
DOTA-OC: [DOTA⁰, D-Phe¹]octreotide,
DOTA-TOC: [DOTA⁰, D-Phe¹, Tyr³]octreotide, edotreotide (INN),
represented by the following formulas:

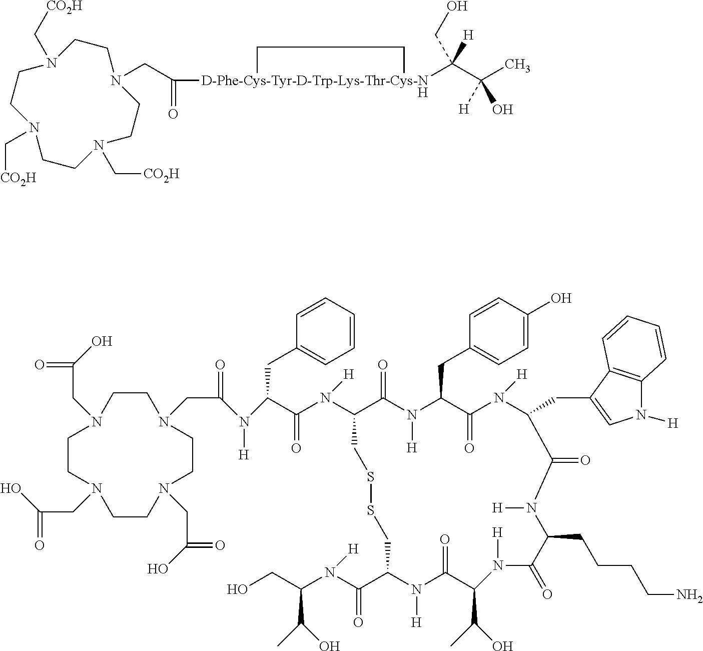

DOTA-NOC: [DOTA⁰, D-Phe¹, ₁-Nal³]octreotide,
DOTA-TATE: [DOTA⁰, D-Phe¹, Tyr³]octreotate, DOTA-Tyr³-Octreotate, DOTA-d-Phe-Cys-Tyr-d-Trp-Lys-Thr-Cys-Thr (cyclo 2,7), oxodotreotide (INN), represented by the following formula:

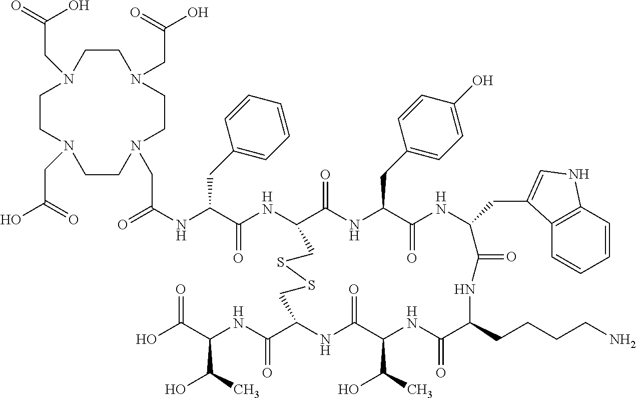 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

DOTA-LAN. [DOTA⁰, D-β-Nal¹]lanreotide,
DOTA-VAP. [DOTA⁰, D-Phe¹, Tyr³]vapreotide.

### Satoreotide Trizoxetan

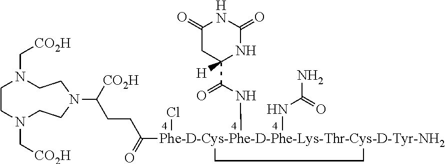

### Satoreotide Tetraxetan

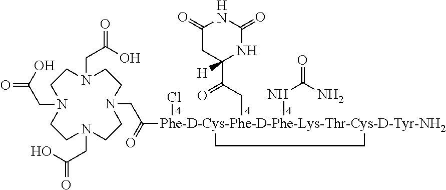

The preferred “cell receptor binding moiety linked to the chelating agent” molecules for the present invention are DOTA-TOC, DOTA-TATE, and Satoreotide tetraxetan, more preferably the molecule is DOTA-TATE.
For the present invention, the preferred complex formed by (or the preferred complex of) the radionuclide and the cell receptor binding moiety linked to the chelating agent according to the present invention is ¹⁷⁷Lu-DOTA-TATE, which is also referred to as Lutetium (177Lu) oxodotreotide (INN), i.e. hydrogen [N-{[4,7,10-tris(carboxylato-κO-methyl)-1,4,7,10-tetraazacyclododecan-1-yl-κ⁴N1,N⁴,N⁷,N¹⁰]acetyl-κP}-D-phenylalanyl-L-cysteinyl-tyrosyl-D-tryptophyl-L-lysyl-L-threonyl-L-cysteinyl-L-threoninato cyclic (2->7)-disulfide(4-)](177Lu)lutetate(1-) and is represented by the following formulas:

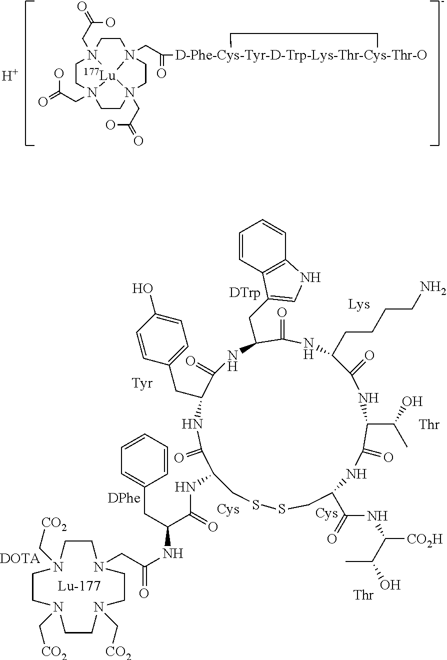

### Further Anti-Cancer Agents

The present invention further provides the combination or combination therapy of the complex formed by the radionuclide ¹⁷⁷Lu (Lutetium-177), and a somatostatin receptor binding peptide linked to the chelating agent as defined herein, or the combination or combination therapy of the pharmaceutical aqueous solution as defined herein, together with one of more therapeutic agents as outlined in the following:

In certain instances, pharmaceutical aqueous solution of the present invention are combined with other therapeutic agents, such as other anti-cancer agents, anti-allergic agents, anti-nausea agents (or anti-emetics), pain relievers, cytoprotective agents, and combinations thereof.

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the pharmaceutical aqueous solution of the present invention include:

Tyrosine kinase inhibitors: Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Vascular Endothelial Growth Factor (VEGF) receptor inhibitors: Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-J][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®), sulfatinib, surufatinib.

Platelet-derived Growth Factor (PDGF) receptor inhibitors: Imatinib (Gleevec®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Quizartinib (AC220, CAS 950769-58-1); Pazopanib (Votrient®); Axitinib (Inlyta®); Sorafenib (Nexavar®); Vargatef (BIBF1120, CAS 928326-83-4); Telatinib (BAY57-9352, CAS 332012-40-5); Vatalanib dihydrochloride (PTK787, CAS 212141-51-0); and Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Fibroblast Growth Factor Receptor (FGFR) Inhibitors: Brivanib alaninate (BMS-582664, (S)-((R)-1-(4-(4-Fluoro-2-methyl-1H-indol-5-yloxy)-5-methylpyrrolo[2,1-J][1,2,4]triazin-6-yloxy)propan-2-yl)2-aminopropanoate); Vargatef (BIBF1120, CAS 928326-83-4); Dovitinib dilactic acid (TKI258, CAS 852433-84-2); 3-(2,6-Dichloro-3,5-dimethoxy-phenyl)-1-{6-[4-(4-ethyl-piperazin-1-yl)-phenylamino]-pyrimidin-4-yl}-1-methyl-urea (BGJ398, CAS 872511-34-7); Danusertib (PHA-739358); and N-[2-[[4-(Diethylamino)butyl]amino]-6-(3,5-dimethoxyphenyl)pyrido[2,3-d]pyrimidin-7-yl]-N′-(1,1-dimethylethyl)-urea (PD173074, CAS 219580-11-7). sulfatinib, surufatinib.

Aurora kinase inhibitors: Danusertib (PHA-739358); N-[4-[[6-Methoxy-7-[3-(4-morpholinyl)propoxy]-4-quinazolinyl]amino]phenyl]benzamide (ZM447439, CAS 331771-20-1); 4-(2-Amino-4-methyl-5-thiazolyl)-N-[4-(4-morpholinyl)phenyl]-2-pyrimidinamine (CYC116, CAS 693228-63-6); Tozasertib (VX680 or MK-0457, CAS 639089-54-6); Alisertib (MLN8237); (N-{2-[6-(4-Cyclobutylamino-5-trifluoromethyl-pyrimidine-2-ylamino)-(1S,4R)-1,2,3,4-tetrahydro-1,4-epiazano-naphthalen-9-yl]-2-oxo-ethyl}-acetamide) (PF-03814735); 4-[[9-Chloro-7-(2,6-difluorophenyl)-5H-pyrimido[5,4-d][2]benzazepin-2-yl]amino]-benzoic acid (MLN8054, CAS 869363-13-3); Cenisertib (R-763); Barasertib (AZD1152); and N-cyclopropyl-N′-[3-[6-(4-morpholinylmethyl)-1H-benzimidazol-2-yl]-1H-pyrazol-4-yl]-urea (AT9283).

Cyclin-Dependent Kinase (CDK) inhibitors: Aloisine A; Alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); Crizotinib (PF-02341066, CAS 877399-52-5); 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); Indisulam (E7070); Roscovitine (CYC202); 6-Acetyl-8-cyclopentyl-5-methyl-2-(5-piperazin-1-yl-pyridin-2-ylamino)-8H-pyrido[2,3-d]pyrimidin-7-one, hydrochloride (PD0332991); Dinaciclib (SCH727965); N-[5-[[(5-tert-Butyloxazol-2-yl)methyl]thio]thiazol-2-yl]piperidine-4-carboxamide (BMS 387032, CAS 345627-80-7); 4-[[9-Chloro-7-(2,6-difluorophenyl)-5H-pyrimido[5,4-d][2]benzazepin-2-yl]amino]-benzoic acid (MLN8054, CAS 869363-13-3); 5-[3-(4,6-Difluoro-1H-benzimidazol-2-yl)-1H-indazol-5-yl]-N-ethyl-4-methyl-3-pyridinemethanamine (AG-024322, CAS 837364-57-5); 4-(2,6-Dichlorobenzoylamino)-1H-pyrazole-3-carboxylic acid N-(piperidin-4-yl)amide (AT7519, CAS 844442-38-2); 4-[2-Methyl-1-(1-methylethyl)-1H-imidazol-5-yl]-N-[4-(methylsulfonyl)phenyl]-2-pyrimidinamine (AZD5438, CAS 602306-29-6); Palbociclib (PD-0332991); and (2R,3R)-3-[[2-[[3-[[S(R)]-S-cyclopropylsulfonimidoyl]-phenyl]amino]-5-(trifluoromethyl)-4-pyrimidinyl]oxy]-2-butanol (BAY 10000394), ribociclib.

Checkpoint Kinase (CHK) inhibitors: 7-Hydroxystaurosporine (UCN-01); 6-Bromo-3-(1-methyl-1H-pyrazol-4-yl)-5-(3R)-3-piperidinyl-pyrazolo[1,5-a]pyrimidin-7-amine (SCH900776, CAS 891494-63-6); 5-(3-Fluorophenyl)-3-ureidothiophene-2-carboxylic acid N-[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7,4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL), and CBP501 ((d-Bpa)sws(d-Phe-F5)(d-Cha)rrrqrr); and (αR)-α-amino-N-[5,6-dihydro-2-(1-methyl-1H-pyrazol-4-yl)-6-oxo-1H-pyrrolo[4,3,2-ef][2,3]benzodiazepin-8-yl]-Cyclohexaneacetamide (PF-0477736).

3-Phosphoinositide-dependent kinase-1 (PDK1 or PDPK1) inhibitors: 7-2-Amino-N-[4-[5-(2-phenanthrenyl)-3-(trifluoromethyl)-1H-pyrazol-1-yl]phenyl]-acetamide (OSU-03012, CAS 742112-33-0); Pyrrolidine-1-carboxylic acid (3-{5-bromo-4-[2-(1H-imidazol-4-yl)-ethylamino]-pyrimidin-2-ylamino}-phenyl)-amide (BX912, CAS 702674-56-4); and 4-Dodecyl-N-1,3,4-thiadiazol-2-yl-benzenesulfonamide (PHT-427, CAS 1191951-57-1).

Protein Kinase C (PKC) activators: Bryostatin I (bryo-1) and Sotrastaurin (AEB071).

B-RAF inhibitors: Regorafenib (BAY73-4506, CAS 755037-03-7); Tuvizanib (AV951, CAS 475108-18-0); Vemurafenib (Zelboraf®, PLX-4032, CAS 918504-65-1); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1H-pyrrolo[2,3-b]pyridine-3-carbonyl)-2,4-difluorophenyl)propane-1-sulfonamide (also known as PLX4720).

C-RAF Inhibitors: Sorafenib (Nexavar®); 3-(Dimethylamino)-N-[3-[(4-hydroxybenzoyl)amino]-4-methylphenyl]-benzamide (ZM336372, CAS 208260-29-1); and 3-(1-cyano-1-methylethyl)-N-[3-[(3,4-dihydro-3-menthyl-4-oxo-6-quinazolinyl)amino]-4-m ethylphenyl]-benzamide (AZ628, CAS 1007871-84-2).

Human Granulocyte colony-stimulating factor (G-CSF) modulators: Filgrastim (Neupogen®); Sunitinib malate (Sutent®); Pegilgrastim (Neulasta®) and Quizartinib (AC220, CAS 950769-58-1).

RET Inhibitors: Sunitinib malate (Sutent®); Vandetanib (Caprelsa®); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Sorafenib (BAY 43-9006); Regorafenib (BAY73-4506, CAS 755037-03-7); and Danusertib (PHA-739358).

FMS-like Tyrosine kinase 3 (FLT3) Inhibitors or CD135: Sunitinib malate (Sutent®); Quizartinib (AC220, CAS 950769-58-1); N-[(1-Methyl-4-piperidinyl)methyl]-3-[3-(trifluoromethoxy)phenyl]-Imidazo[1,2-b]pyridazin-6-amine sulfate (SGI-1776, CAS 1173928-26-1); and Vargatef (BIBF1120, CAS 928326-83-4).

c-KIT Inhibitors: Pazopanib (Votrient®); Dovitinib dilactic acid (TK1258, CAS 852433-84-2); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Masitinib (Masivet®); Regorafenib (BAY73-4506, CAS 755037-03-7); Tivozanib (AV951, CAS 475108-18-0); Vatalanib dihydrochloride (PTK787, CAS 212141-51-0); Telatinib (BAY57-9352, CAS 332012-40-5); Foretinib (GSK1363089, formerly XL880, CAS 849217-64-7); Sunitinib malate (Sutent®); Quizartinib (AC220, CAS 950769-58-1); Axitinib (Inlyta®); Dasatinib (BMS-345825); and Sorafenib (Nexavar®).

Bcr/Abl kinase inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]-acetamide (PF-03814735, CAS 942487-16-3).

IGF-1R inhibitors: Linsitnib (OSI-906); [7-[trans-3-[(Azetidin-1-yl)methyl]cyclobutyl]-5-(3-benzyloxyphenyl)-7H-pyrrolo[2,3-d]pyrimidin-4-yl]amine (AEW541, CAS 475488-34-7); [5-(3-Benzyloxyphenyl)-7-[trans-3-[(pyrrolidin-1-yl)methyl]cyclobutyl]-7H-pyrrolo[2,3-d]pyrimidin-4-yl]amine (ADW742 or GSK552602A, CAS 475488-23-4); (2-[[3-Bromo-5-(1,1-dimethylethyl)-4-hydroxyphenyl]methylene]-propanedinitrile (Tyrphostin AG1024, CAS 65678-07-1); 4-[[(2S)-2-(3-Chlorophenyl)-2-hydroxyethyl]amino]-3-[7-methyl-5-(4-morpholinyl)-1H-benzimidazol-2-yl]-2(1H)-pyridinone (BMS536924, CAS 468740-43-4); 4-[2-[4-[[(2S)-2-(3-Chlorophenyl)-2-hydroxyethyl]amino]-1,2-dihydro-2-oxo-3-pyridinyl]-7-methyl-1H-benzimidazol-5-yl]-1-piperazinepropanenitrile (BMS554417, CAS 468741-42-6); (2S)-1-[4-[(5-Cyclopropyl-1H-pyrazol-3-yl)amino]pyrrolo[2,1-][1,2,4]triazin-2-yl]-N-(6-fluoro-3-pyridinyl)-2-methyl-2-pyrrolidinecarboxamide (BMS754807, CAS 1001350-96-4); Picropodophyllotoxin (AXL1717); and Nordihydroguareacetic acid.

IGF-1R antibodies: Figitumumab (CP751871); Cixutumumab (IMC-A12); Ganitumab (AMG-479); Robatumumab (SCH-717454); Dalotuzumab (MK0646); R1507 (available from Roche); BIIB022 (available from Biogen); and MEDI-573 (available from MedImmune).

MET inhibitors: Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)-N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)-N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin-3-yl]thio]-quinoline (SGX523, CAS 1022150-57-7); and (3Z)-5-[[(2,6-Dichlorophenyl)methyl]sulfonyl]-3-[[33,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

Epidermal growth factor receptor (EGFR) inhibitors: Erlotinib hydrochloride (Tarceva®), Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5p,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

EGFR antibodies: Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1).

mTOR inhibitors: Temsirolimus (Torisel®); Ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1); and N-[4-[[[3-[(3,5-dimethoxyphenyl)amino]-2-quinoxalinyl]amino]sulfonyl]phenyl]-3-methoxy-4-methyl-benzamide (XL765, also known as SAR245409); and (1r,4r)-4-(4-amino-5-(7-methoxy-1H-indol-2-yl)imidazo[1,5-f][,2,4]triazin-7-yl)cyclohexanecarboxylic acid (OSI-027).

Mitogen-activated protein kinase (MEK) inhibitors: XL-518 (also known as GDC-0973, Cas No. 1029872-29-4, available from ACC Corp.); Selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO2003077914); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione](also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); Trametinib dimethyl sulfoxide (GSK-1120212, CAS 1204531-25-80); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

Alkylating agents: Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Aromatase inhibitors: Exemestane (Aromasin®); Letrozole (Femara®); and Anastrozole (Arimidex®).

Topoisomerase I inhibitors: Irinotecan (Camptosar®); Topotecan hydrochloride (Hycamtin®); and 7-Ethyl-10-hydroxycampothecin (SN38).

Topoisomerase IIinhibitors: Etoposide (VP-16 and Etoposide phosphate, Toposar®, VePesid® and Etopophos®); Teniposide (VM-26, Vumon®); and Tafluposide.

DNA Synthesis inhibitors: Capecitabine (Xeloda®); Gemcitabine hydrochloride (Gemzar®); Nelarabine ((2R,3S,4R,5R)-2-(2-amino-6-methoxy-purin-9-yl)-5-(hydroxymethyl)oxolane-3,4-diol, Arranon® and Atriance®); and Sapacitabine (1-(2-cyano-2-deoxy-β-D-arabinofuranosyl)-4-(palmitoylamino)pyrimidin-2(1H)-one).

Folate Antagonists or Antifolates: Trimetrexate glucuronate (Neutrexin®); Piritrexim isethionate (BW201U); Pemetrexed (LY231514); Raltitrexed (Tomudex®); and Methotrexate (Rheumatrex®, Trexal®).

Immunomodulators: Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon γ, CAS 951209-71-5, available from IRX Therapeutics).

G-Protein-coupled Somatostain receptors Inhibitors: Octreotide (also known as octreotide acetate, Sandostatin® and Sandostatin LAR®); Lanreotide acetate (CAS 127984-74-1); Seglitide (MK678); Vapreotide acetate (Sanvar®); and Cyclo(D-Trp-Lys-Abu-Phe-MeAla-Tyr)(BIM23027).

Interleukin-11 and Synthetic Interleukin-11 (IL-11): Oprelvekin (Neumega®).

Erythropoietin and Synthetic erythropoietin: Erythropoietin (Epogen® and Procrit®); Darbepoetin alfa (Aranesp®); Peginesatide (Hematide®); and EPO covalently linked to polyethylene glycol (Micera®).

Histone deacetylase (HDAC) inhibitors: Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl](Cyl-1); Cyclo[(αS,2S)-α-amino-η-oxo-2-oxiraneoctanoyl-O-methyl-D-tyrosyl-L-isoleucyl-(2S)-2-piperidinecarbonyl](Cyl-2); Cyclic[L-alanyl-D-alanyl-(2S)---oxo-L-α-aminooxiraneoctanoyl-D-prolyl](HC-toxin); Cyclo[(αS,2S)-α-amino-η-oxo-2-oxiraneoctanoyl-D-phenylalanyl-L-leucyl-(2S)-2-piperidinecarbonyl](WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); and Depudecin (4,5:8,9-dianhydro-1,2,6,7,11-pentadeoxy-D-threo-D-ido-Undeca-1,6-dienitol).

Biologic response modifiers: Include therapeutics such as interferons, interleukins, colony-stimulating factors, monoclonal antibodies, vaccines (therapeutic and prophylactic), gene therapy, and nonspecific immunomodulating agents. Interferon alpha (Intron®, Roferson®-A); Interferon beta; Interferon gamma; Interleukin-2 (IL-2 or aldesleukin, Proleukin®); Filgrastim (Neupogen®); Sargramostim (Leukine®); Erythropoietin (epoetin); Interleukin-11 (oprelvekin); Imiquimod (Aldara®); Lenalidomide (Revlimid®); Rituximab (Rituxan®); Trastuzumab (Herceptin®); Bacillus calmette-guerin (theraCys® and TICE® BCG); Levamisole (Ergamisol®); and Denileukin diftitox (Ontak®).

Plant Alkaloids: Paclitaxel (Taxol and Onxal™); Paclitaxel protein-bound (Abraxane®); Vinblastine (also known as vinblastine sulfate, vincaleukoblastine and VLB, Alkaban-AQ® and Velban®); Vincristine (also known as vincristine sulfate, LCR, and VCR, Oncovin® and Vincasar Pfs®); and Vinorelbine (Navelbine®).

Taxane anti-neoplastic agents: Paclitaxel (Taxol®); Docetaxel (Taxotere®); Cabazitaxel (Jevtana®, 1-hydroxy-70,10β-dimethoxy-9-oxo-5β,20-epoxytax-11-ene-2α,4,13α-triyl-4-acetate-2-benzoate-13-[(2R,3S)-3-{[(tert-butoxy)carbonyl]amino}-2-hydroxy-3-phenylpropanoate); and Larotaxel ((2α, 3,ξ4α, 5β, 7α, 10β, 13α)-4,10-bis(acetyloxy)-13-({(2R,3S)-3-[(tert-butoxycarbonyl) amino]-2-hydroxy-3-phenylpropanoyl}oxy)-1-hydroxy-9-oxo-5,20-epoxy-7,19-cyclotax-11-en-2-yl benzoate).

Heat Shock Protein (HSP) inhibitors: Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (B11B021 or CNF2024, CAS 848695-25-0); trans-4-[[2-(Aminocarbonyl)-5-[4,5,6,7-tetrahydro-6,6-dimethyl-4-oxo-3-trifluoromethyl)-1H-indazol-1-yl]phenyl]amino]cyclohexyl glycine ester (SNX5422 or PF04929113, CAS 908115-27-5); and 17-Dimethylaminoethylamino-17-demethoxygeldanamycin (17-DMAG).

Thrombopoietin (TpoR) agonists: Eltrombopag (SB497115, Promacta® and Revolade®); and Romiplostim (Nplate®).

Demethylating agents: 5-Azacitidine (Vidaza®); and Decitabine (Dacogen®).

Cytokines: Interleukin-2 (also known as aldesleukin and IL-2, Proleukin®); Interleukin-11 (also known as oprevelkin, Neumega®); and Alpha interferon alfa (also known as IFN-alpha, Intron® A, and Roferon-A®).

17 a-hydroxylase/C17,20 lyase (CYP17A1) inhibitors: Abiraterone acetate (Zyitga®).

Miscellaneous cytotoxic agents: Arsenic trioxide (Trisenox®); Asparaginase (also known as L-asparaginase, Erwinia L-asparaginase, Elspar® and Kidrolase®); and Asparaginase Erwinia Chrysanthemi (Erwinaze®).

C-C Chemokine receptor 4 (CCR4) Antibody: Mogamulizumab (Potelligent®)

CD20 antibodies: Rituximab (Riuxan® and MabThera®); and Tositumomab (Bexxar®); and Ofatumumab (Arzerra®).

CD20 Antibody Drug Conjugates: Ibritumomab tiuxetan (Zevalin®); and Tositumomab,

CD22 Antibody Drug Conjugates: Inotuzumab ozogamicin (also referred to as CMC-544 and WAY-207294, available from Hangzhou Sage Chemical Co., Ltd.)

CD30 mAb-cytotoxin Conjugates: Brentuximab vedotin (Adcetrix®);

CD33 Antibody Drug Conjugates: Gemtuzumab ozogamicin (Mylotarg®),

CD40 antibodies: Dacetuzumab (also known as SGN-40 or huS2C6, available from Seattle Genetics, Inc),

CD52 antibodies: Alemtuzumab (Campath®),

Anti-CS1 antibodies: Elotuzumab (HuLuc63, CAS No. 915296-00-3)

CTLA-4 antibodies: Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

TPH inhibitors: telotristat

PARP (poly ADP ribose polymerase) inhibitors: olaparib (Lynparza), rucaparib (Rubraca), Niraparib (Zeluja), Talazoparib, Veliparib.

In particular, the present invention provides the combination or combination therapy of the complex formed by the radionuclide ¹⁷⁷Lu (Lutetium-177), and a somatostatin receptor binding peptide linked to the chelating agent as defined herein, or the combination or combination therapy of the pharmaceutical aqueous solution as defined herein, together with one of more therapeutic agents selected from the group consisting of octreotide, lanreotide, vaproreotide, pasireotide, satoreotide, everolimus, temozolomide, telotristat, sunitinib, sulfatinib, ribociclib, entinostat, pazopanib and olaparib.

### Methods of Treating Cancer

In one aspect, the disclosure relates to treatment of a subject in vivo using a combination comprising therapeutic agents disclosed herein, or a composition or formulation comprising a combination disclosed herein, such that growth of cancerous tumors is inhibited or reduced.

In some embodiments, the PD-1 inhibitor, LAG-3 inhibitor, TIM-3 inhibitor, GITR agonist, TGF-β inhibitor, an IL-15/IL15RA complex, is administered or used in accordance with a dosage regimen disclosed herein.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject a combination comprising three or more (e.g., four or more) therapeutic agents disclosed herein, or a composition or formulation comprising a combination disclosed herein, e.g., in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial, bladder cells), prostate, CNS (e.g., brain, neural or glial cells), skin, pancreas, and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. Metastatic lesions of the aforementioned cancers can also be treated or prevented using the methods and compositions of the invention.

As used herein, the term “subject” is intended to include human and non-human animals.

The combination therapies described herein can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

When administered in combination, the therapeutic agent can be administered in an amount or dose that is higher or lower than, or the same as, the amount or dosage of each agent used individually, e.g., as a monotherapy. In certain embodiments, the administered amount or dosage of the therapeutic agent is lower (e.g., at least 20%, at least 30%, at least 40%, or at least 50%) than the amount or dosage of each agent used individually, e.g., as a monotherapy. In other embodiments, the amount or dosage of the therapeutic agent that results in a desired effect (e.g., treatment of cancer) is lower (e.g., at least 20%, at least 30%, at least 40%, or at least 50% lower).

### Pharmaceutical Compositions

In another aspect, the present invention provides compositions, e.g., pharmaceutically acceptable compositions, which includes one or more of, e.g., two, three, four, five, six, seven, eight, or more of, a therapeutic agent described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In certain embodiments, the mode of administration is parenteral (e.g., intravenous, subcutaneous, intraperitoneal, or intramuscular). In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, a PD-1 inhibitor, a LAG-3 inhibitor, a TIM-3 inhibitor, a GITR agonist, a TGF-β inhibitor, an IL-15/IL-15RA complex, or any combination thereof, can be formulated into a formulation (e.g., a dose formulation or dosage form) suitable for administration (e.g., intravenous administration) to a subject as described herein.

In some embodiments, a PD-1 inhibitor (e.g., anti-PD-1 antibody molecule) or a composition described herein can be formulated into a formulation (e.g., a dose formulation or dosage form) suitable for administration (e.g., intravenous administration) to a subject as described herein.

In certain embodiments, the formulation is a drug substance formulation. In other embodiments, the formulation is a lyophilized formulation, e.g., lyophilized or dried from a drug substance formulation. In other embodiments, the formulation is a reconstituted formulation, e.g., reconstituted from a lyophilized formulation. In other embodiments, the formulation is a liquid formulation. In some embodiments, the formulation (e.g., drug substance formulation) comprises a PD-1 inhibitor, a LAG-3 inhibitor, a TIM-3 inhibitor, a GITR agonist, a TGF-β inhibitor, an IL-15/IL-15RA complex, or any combination thereof.

In some embodiments, the formulation is a drug substance formulation. In some embodiments, the formulation (e.g., drug substance formulation) comprises the PD-1 inhibitor (e.g., the anti-PD-1 antibody molecule) and a buffering agent.

In some embodiments, the formulation (e.g., drug substance formulation) comprises a PD-1 inhibitor (e.g., an anti-PD-1 antibody molecule) present at a concentration of 10 to 50 mg/mL, e.g., 15 to 50 mg/mL, 20 to 45 mg/mL, 25 to 40 mg/mL, 30 to 35 mg/mL, 25 to 35 mg/mL, or 30 to 40 mg/mL, e.g., 15 mg/mL, 20 mg/mL, 25 mg/mL, 30 mg/mL, 33.3 mg/mL, 35 mg/mL, 40 mg/mL, 45 mg/mL, or 50 mg/mL. In certain embodiments, the PD-1 inhibitor (e.g., the anti-PD-1 antibody molecule) is present at a concentration of 30 to 35 mg/mL, e.g., 33.3 mg/mL.

In some embodiments, the formulation (e.g., drug substance formulation) comprises a buffering agent comprising histidine (e.g., a histidine buffer). In certain embodiments, the buffering agent (e.g., histidine buffer) is present at a concentration of 1 mM to 20 mM, e.g., 2 mM to 15 mM, 3 mM to 10 mM, 4 mM to 9 mM, 5 mM to 8 mM, or 6 mM to 7 mM, e.g., 1 mM, 2 mM, 3 mM, 4 mM, 5 mM, 6 mM, 6.7 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some embodiments, the buffering agent (e.g., histidine buffer) is present at a concentration of 6 mM to 7 mM, e.g., 6.7 mM. In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises histidine at a concentration of 6 mM to 7 mM (e.g., 6.7 mM) and has a pH of 5 to 6 (e.g., 5.5).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., drug substance formulation) further comprises a carbohydrate. In certain embodiments, the carbohydrate is sucrose. In some embodiments, the carbohydrate (e.g., sucrose) is present at a concentration of 50 mM to 150 mM, e.g., 25 mM to 150 mM, 50 mM to 100 mM, 60 mM to 90 mM, 70 mM to 80 mM, or 70 mM to 75 mM, e.g., 25 mM, 50 mM, 60 mM, 70 mM, 73.3 mM, 80 mM, 90 mM, 100 mM, or 150 mM. In some embodiments, the formulation comprises a carbohydrate or sucrose present at a concentration of 70 mM to 75 mM, e.g., 73.3 mM.

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a carbohydrate or sucrose present at a concentration of 70 mM to 75 mM, e.g., 73.3 mM.

In some embodiments, the formulation is a drug substance formulation. In some embodiments, the formulation (e.g., drug substance formulation) comprises a PD-1 inhibitor, a LAG-3 inhibitor, a TIM-3 inhibitor, a GITR agonist, a TGF-β inhibitor, an IL-15/IL-15RA complex, or any combination thereof and a buffering agent.

In some embodiments, the formulation (e.g., drug substance formulation) further comprises a surfactant. In certain embodiments, the surfactant is polysorbate 20. In some embodiments, the surfactant or polysorbate 20) is present at a concentration of 0.005% to 0.025% (w/w), e.g., 0.0075% to 0.02% or 0.01% to 0.015% (w/w), e.g., 0.005%, 0.0075%, 0.01%, 0.013%, 0.015%, or 0.02% (w/w). In some embodiments, the formulation comprises a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); a carbohydrate or sucrose present at a concentration of 70 mM to 75 mM, e.g., 73.3 mM; and a surfactant or polysorbate 20 present at a concentration of 0.01% to 0.015%, e.g., 0.013% (w/w).

In some embodiments, the formulation (e.g., drug substance formulation) comprises a PD-1 inhibitor (e.g., an anti-PD-1 antibody molecule) present at a concentration of 33.3 mg/mL; a buffering agent that comprises histidine at a concentration of 6.7 mM and has a pH of 5.5; sucrose present at a concentration of 73.3 mM; and polysorbate 20 present at a concentration of 0.013% (w/w).

In some embodiments, the formulation is a lyophilized formulation. In certain embodiments, the lyophilized formulation is lyophilized from a drug substance formulation described herein. For example, 2 to 5 mL, e.g., 3 to 4 mL, e.g., 3.6 mL, of the drug substance formulation described herein can be filled per container (e.g., vial) and lyophilized.

In certain embodiments, the formulation is a reconstituted formulation. For example, a reconstituted formulation can be prepared by dissolving a lyophilized formulation in a diluent such that the protein is dispersed in the reconstituted formulation. In some embodiments, the lyophilized formulation is reconstituted with 0.5 mL to 2 mL, e.g., 1 mL, of water or buffer for injection. In certain embodiments, the lyophilized formulation is reconstituted with 1 mL of water for injection, e.g., at a clinical site.

In some embodiments, the formulation (e.g., reconstituted formulation) comprises a PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a MEK inhibitor, an IL-15/IL-15RA complex, an IL-1β inhibitor, or any combination thereof, and a buffering agent.

In some embodiments, the formulation (e.g., reconstituted formulation) comprises a PD-1 inhibitor (e.g., an anti-PD-1 antibody molecule) present at a concentration of 20 mg/mL to 200 mg/mL, e.g., 50 mg/mL to 150 mg/mL, 80 mg/mL to 120 mg/mL, or 90 mg/mL to 110 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In certain embodiments, the PD-1 inhibitor (e.g., the anti-PD-1 antibody molecule) is present at a concentration of 80 to 120 mg/mL, e.g., 100 mg/mL.

In some embodiments, the formulation (e.g., reconstituted formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., reconstituted formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., reconstituted formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., reconstituted formulation) comprises a PD-1 inhibitor (e.g., an anti-PD-1 antibody molecule) present at a concentration of 100 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In some embodiments, the formulation is reconstituted such that an extractable volume of at least 1 mL (e.g., at least 1.5 mL, 2 mL, 2.5 mL, or 3 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

In certain embodiments, the formulation is a liquid formulation. In some embodiments, the liquid formulation is prepared by diluting a drug substance formulation described herein. For example, a drug substance formulation can be diluted, e.g., with 10 to 30 mg/mL (e.g., 25 mg/mL) of a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In some embodiments, the formulation (e.g., liquid formulation) comprises a PD-1 inhibitor (e.g., an anti-PD-1 antibody molecule) present at a concentration of 5 mg/mL to 50 mg/mL, e.g., 10 mg/mL to 40 mg/mL, 15 mg/mL to 35 mg/mL, or 20 mg/mL to 30 mg/mL, e.g., 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. In certain embodiments, the PD-1 inhibitor (e.g., the anti-PD-1 antibody molecule) is present at a concentration of 20 to 30 mg/mL, e.g., 25 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., liquid formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., liquid d formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., liquid formulation) comprises a PD-1 inhibitor (e.g., an anti-PD-1 antibody molecule) present at a concentration of 25 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In certain embodiments, 1 mL to 10 mL (e.g., 2 mL to 8 mL, 3 mL to 7 mL, or 4 mL to 5 mL, e.g., 3 mL, 4 mL, 4.3 mL, 4.5 mL, 5 mL, or 6 mL) of the liquid formulation is filled per container (e.g., vial). In other embodiments, the liquid formulation is filled into a container (e.g., vial) such that an extractable volume of at least 2 mL (e.g., at least 3 mL, at least 4 mL, or at least 5 mL) of the liquid formulation can be withdrawn per container (e.g., vial). In certain embodiments, the liquid formulation is diluted from the drug substance formulation and/or extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 50 mg to 150 mg, e.g., 80 mg to 120 mg, 90 mg to 110 mg, 100 mg to 120 mg, 100 mg to 110 mg, 110 mg to 120 mg, or 110 mg to 130 mg, of the PD-1 inhibitor (e.g., the anti-PD-1 antibody molecule), is present in the container (e.g., vial).

Other exemplary buffering agents that can be used in the formulation described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulation described herein may also contain a tonicity agent, e.g., sodium chloride, and/or a stabilizing agent, e.g., an amino acid (e.g., glycine, arginine, methionine, or a combination thereof).

The therapeutic agents, e.g., inhibitors, antagonist or binding agents, can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m 2, preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic agent or compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the disclosure by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic agent is 0.1-30 mg/kg, more preferably 1-25 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

As another example, non-limiting range for a therapeutically or prophylactically effective amount of an antibody molecule is 200-500 mg, more preferably 300-400 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks. While not wishing to be bound by theory, in some embodiments, flat or fixed dosing can be beneficial to patients, for example, to save drug supply and to reduce pharmacy errors.

In some embodiments, the clearance (CL) of the anti-PD-1 antibody molecule is from about 6 to 16 mL/h, e.g., about 7 to 15 mL/h, about 8 to 14 mL/h, about 9 to 12 mL/h, or about 10 to 11 mL/h, e.g., about 8.9 mL/h, 10.9 mL/h, or 13.2 mL/h.

In some embodiments, the exponent of weight on CL of the anti-PD-1 antibody molecule is from about 0.4 to 0.7, about 0.5 to 0.6, or 0.7 or less, e.g., 0.6 or less, or about 0.54.

In some embodiments, the volume of distribution at steady state (Vss) of the anti-PD-1 antibody molecule is from about 5 to 10 V, e.g., about 6 to 9 V, about 7 to 8 V, or about 6.5 to 7.5 V, e.g., about 7.2 V.

In some embodiments, the half-life of the anti-PD-1 antibody molecule is from about 10 to 30 days, e.g., about 15 to 25 days, about 17 to 22 days, about 19 to 24 days, or about 18 to 22 days, e.g., about 20 days.

In some embodiments, the Cmin (e.g., for a 80 kg patient) of the anti-PD-1 antibody molecule is at least about 0.4 μg/mL, e.g., at least about 3.6 μg/mL, e.g., from about 20 to 50 μg/mL, e.g., about 22 to 42 μg/mL, about 26 to 47 μg/mL, about 22 to 26 μg/mL, about 42 to 47 μg/mL, about 25 to 35 μg/mL, about 32 to 38 μg/mL, e.g., about 31 μg/mL or about 35 μg/mL. In one embodiment, the Cmin is determined in a patient receiving the anti-PD-1 antibody molecule at a dose of about 400 mg once every four weeks. In another embodiment, the Cmin is determined in a patient receiving the anti-PD-1 antibody molecule at a dose of about 300 mg once every three weeks. In some embodiments, In certain embodiments, the Cmin is at least about 50-fold higher, e.g., at least about 60-fold, 65-fold, 70-fold, 75-fold, 80-fold, 85-fold, 90-fold, 95-fold, or 100-fold, e.g., at least about 77-fold, higher than the EC50 of the anti-PD-1 antibody molecule, e.g., as determined based on IL-2 change in an SEB ex-vivo assay. In other embodiments, the Cmin is at least 5-fold higher, e.g., at least 6-fold, 7-fold, 8-fold, 9-fold, or 10-fold, e.g., at least about 8.6-fold, higher than the EC90 of the anti-PD-1 antibody molecule, e.g., as determined based on IL-2 change in an SEB ex-vivo assay.

The antibody molecule can be administered by intravenous infusion at a rate of more than 20 mg/min, e.g., 20-40 mg/min, and typically greater than or equal to 40 mg/min to reach a dose of about to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

LUTATHERA (lutetium Lu 177 dotatate) is a radiolabeled somatostatin analog. The drug substance lutetium Lu 177 dotatate is a cyclic peptide linked with the covalently bound chelator 1,4,7,10-tetraazacyclododecane-1,4,7,10-tetraacetic acid to a radionuclide.

Lutetium Lu 177 dotatate is described as lutetium (Lu 177)-N-[(4,7,10-Tricarboxymethyl-1,4,7,10-tetraazacyclododec-1-yl) acetyl]-Dphenylalanyl-L-cysteinyl-L-tyrosyl-D-tryptophanyl-L-lysyl-L-threoninyl-L-cysteinyl-L-threonine-cyclic (2-7) disulfide. The molecular weight is 1609.6 Daltons and the structural formula is as follows:

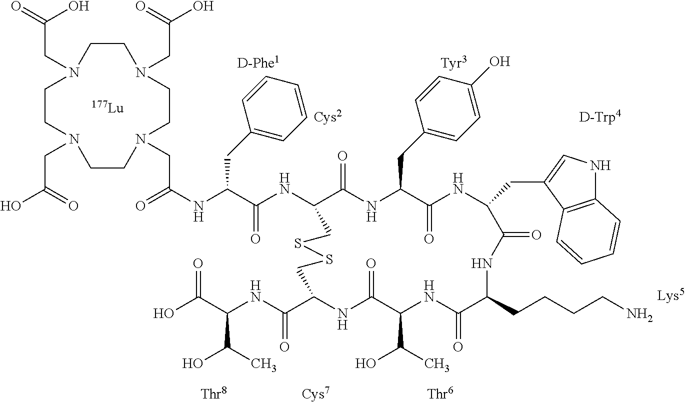

LUTATHERA (lutetium Lu 177 dotatate) 370 MBq/mL (10 mCi/mL) Injection is a sterile, clear, colorless to slightly yellow solution for intravenous use. Each single-dose vial contains acetic acid (0.48 mg/mL), sodium acetate (0.66 mg/mL), gentisic acid (0.63 mg/mL), sodium hydroxide (0.65 mg/mL), ascorbic acid (2.8 mg/mL), diethylene triamine pentaacetic acid (0.05 mg/mL), sodium chloride (6.85 mg/mL), and Water for Injection (ad 1 mL). The pH range of the solution is 4.5 to 6.

LUTATHERA Injection containing 370 MBq/mL (10 mCi/ml) of lutetium Lu 177 dotatate is a sterile, preservative-free and clear, colorless to slightly yellow solution for intravenous use supplied in a colorless Type I glass 30 mL single-dose vial containing 7.4 GBq (200 mCi)±10% of lutetium Lu 177 dotatate at the time of injection (NDC #69488-003-01). The solution volume in the vial is adjusted from 20.5 mL to 25 mL to provide a total of 7.4 GBq (200 mCi) of radioactivity.

The product vial is in a lead shielded container placed in a plastic sealed container (NDC #69488-003-01). The product is shipped in a Type A package (NDC #69488-003-70).

Store below 25° C. (77° F.).

The shelf life is 72 hours. Discard appropriately at 72 hours

### Kits

A combination of therapeutic agents disclosed herein can be provided in a kit. The therapeutic agents are generally provided in a vial or a container. As appropriate, the therapeutic agents can be in liquid or dried (e.g., lyophilized) form. The kits can comprise two or more (e.g., three, four, five, or all) of the therapeutic agents of a combination disclosed herein. In some embodiments, the kit further contains a pharmaceutically acceptable diluent. The therapeutic agents can be provided in the kit in the same or separate formulations (e.g., as mixtures or in separate containers). The kits can contain aliquots of the therapeutic agents that provide for one or more doses. If aliquots for multiple administrations are provided, the doses can be uniform or varied. Varied dosing regimens can be escalating or decreasing, as appropriate. The dosages of the therapeutic agents in the combination can be independently uniform or varying. The kit can include one or more other elements including: instructions for use; other reagents, e.g., a label, or an agent useful for chelating, or otherwise coupling, a therapeutic agent to a label or therapeutic agent, or a radioprotective composition; devices or other materials for preparing the antibody for administration; pharmaceutically acceptable carriers; and devices or other materials for administration to a subject.

### EXAMPLES

The advanced therapeutic effect of the combination of a PRRT with an I-O agent in the treatment of a NET tumor can be demonstrated using the study drug as exemplarily described in Example 1, administered as exemplarily described in Example 2, in a clinical trial as described in Example 3. The following the NET tumor may be small cell lung cancer (SCLC) or pulmonary NET (pNET). The clinical trial demonstrates that the combination of ¹⁷⁷Lu-DOTA⁰-Tyr³-Octreotate and an I-O therapeutic agent is safe and tolerable, and provides PFS benefit compared to observation alone in the maintenance setting in patients with SCLC or pNET and no disease progression after first-line platinum-based chemotherapy.

### Example 1: Study Drug Information

### ¹⁷Lu-DOTA⁰-Tyr³-Octreotate

¹⁷⁷Lu-DOTA⁰-Tyr³-Octreotate is a radiopharmaceutical solution for infusion supplied as a ready-to-use product. No manipulation of the product in needed at the clinical site. 177Lu-DOTA0-Tyr3-Octreotate is manufactured in centralized GMP facilities, and undergoes QC testing before drug supply.

The product is manufactured and supplied to the clinical sites in monodose vials. One vial, for one administration, contains 7.4 GBq (200 mCi) of ¹⁷⁷Lu-DOTA⁰-Tyr³-Octreotate at calibration time (the time of infusion) in a formulation solution of 22 to 25 mL. The variability of the volume depends on the time between the calibration date and the production date. The product will be shipped and calibrated for use at 24 h or 48 h after production in a centralized GMP facility. The calibration time of a dose depends on the distance from the manufacturing facility to the clinical sites. The amount of administered radioactivity, 7.4 GBq (±10%), is specified at the time of infusion.

Chemical-physical properties of each dose are listed in the following table.

TABLE
¹⁷⁷Lu-DOTA⁰-Tyr³-Octreotate Infusion Solution Composition.
Composition of drug product per mL

| | Quantity | | |
|---|---|---|---|
| Property/Component | (Unit/mL) | | Function |
| ¹⁷⁷Lu-DOTA⁰-Tyr³-Octreotate | 370 MBq/mL | | Drug Substance |
| (volumetric activity) | at t_{c} (EOP) | | |
| X-DOTA⁰-Tyr³-Octreotate | 10 | μg/mL | Total peptide |
| | | | content |
| Specific Activity | ≥53 GBq/μmol | | NA |
| (GBq/Total peptide) | at EOP | | |
| Excipients | | | |
| Acetic acid | 0.48 | mg/mL | pH adjuster |
| Sodium acetate | 0.66 | mg/mL | pH adjuster |
| Gentisic acid | 0.63 | mg/mL | RSE |
| Ascorbic acid | 2.80 | mg/mL | RSE |
| DTPA | 0.05 | mg/mL | Sequestering agent |
| Sodium chloride (NaCl) | 6.85 | mg/mL | Isotonizing agent |
| Sodium hydroxide (NaOH) | 0.64 | mg/mL | pH adjuster |

| | | |
|---|---|---|
| Water for injection | Ad 1 mL | Solvent |

EOP: End of Production = t₀ = activity measurement of the first vial = calibration time t_{c}
RSE: Radiation Stability Enhancer

The study drug is For a 74 GBq batch size (2 Ci batch size) a ¹⁷⁷ LuCl₃ solution, about 74 GBq in HCl, is mixed together with a DOTA-Tyr³-Octreotate (about 2 mg) solution, and a Reaction Buffer solution, containing an antioxidant agent (and stabilizator against radiolytic regradation) (i.e. Gentisic acid, about 157 mg) and a buffer system (i.e. Acetate buffer system), resulting in a total of about 5.5 mL solution, which is used for radiolabelling that occurs at a temperature of about 90 to about 98° C. within less than 15 minutes.

The synthesis is carried out using a single use disposable kit cassette installed on the front of the synthesis module which contains the fluid pathway (tubing), reactor vial and sealed reagent vials.

The obtained mother solution is diluted with a solution containing a chelating agent (i.e. DTPA) an antioxidant agent (i.e. Ascorbic acid), sodium hydroxide and sodium chloride and, then, sterile filtered through 0.2 μM to give the ready-to-use solution as described above with a pH of 5.2-5.3. Finally, the solution is dispensed in volumes of from 20.5 to 25.0 mL into sterile vials. The stoppered vials are enclosed within lead containers for protective shielding.

Treatment with ¹⁷⁷Lu-DOTA⁰-Tyr³-Octreotate will consist of a cumulative dose of 29.6 GBq (800mCi) with the dosing equally divided among 4 administrations of ¹⁷⁷Lu-DOTA⁰-Tyr³-Octreotate at 8±1-weeks intervals.

### I-O Therapeutic Agent (an Antibody)

### DESCRIPTION

| | |
|---|---|
| Product name | Antibody Injection, e.g. 100 mg/vial (10 mg/mL) |
| Product | Packaging: Vials assembled into dispensing boxes |
| description and | containing e.g. 5 vials of 100 mg antibody |
| Packaging | Vials: 10 cc Type I glass vial, 20 mm stopper and seal |
| | Appearance: Clear to opalescent, colorless to pale |
| | yellow liquid, light (few) particulates may be present. |
| Product | Each vial contains antibody e.g. 100 mg active |
| Ingredients | |

### Preparation

Withdraw the required volume of antibody solution and transfer into an intravenous container.

Dilute the antibody solution with either 0.9% Sodium Chloride Injection, USP or 5% Dextrose Injection, USP to prepare an infusion with a final concentration ranging from 1 mg/mL to 10 mg/mL. For dilution, the antibody injection may either be added to an empty infusion container and then further diluted by addition of NS or D5W, or the antibody injection may be added directly to an appropriate volume of NS or D5W in a pre-filled infusion container.

Mix diluted solution by gentle inversion. Do not shake.

### Storage of Infusion

The product does not contain a preservative. After preparation, store the antibody infusion either:
at room temperature for no more than 4 hours from the time of preparation. This includes room temperature storage of the infusion in the IV container and time for administration of the infusion
OR
under refrigeration at 2° C. to 8° C. (36° F. to 46° F.) for no more than 24 hours from the time of
infusion preparation.
Do not freeze.

### Example 2: Study Drug Administration

### 1⁷⁷Lu-DOTA⁰-Tyr³-Octreotate

¹⁷⁷Lu-DOTA⁰-Tyr³-Octreotate will be administered every 8 weeks. The first dose of ¹⁷⁷Lu-DOTA⁰-Tyr³-Octreotate will be given two weeks after the first administration of antibody. Each dose is infused over 30 minutes. On the day of ¹⁷⁷Lu-DOTA⁰-Tyr³-Octreotate infusion, an intravenous bolus of anti-emetics will be given (suggested options: ondansetron (8 mg), granisetron (3 mg), or tropisetron (5 mg)). Administration of ¹⁷⁷Lu-DOTA⁰-Tyr³-Octreotate may be given one day earlier or delayed up to 1 week due to holidays, inclement weather, conflicts, or similar reasons. Prednisone should be avoided as preventive anti-emetic treatment due to potential-negative effect on anti-PD-1 therapy. In case of nausea or vomiting despite the use of aforementioned anti-emetic, patients can be treated with other anti-emetic medications at the discretion of the treating physician.

Concurrent amino acids are given with each dose of ¹⁷⁷Lu-DOTA⁰-Tyr³-Octreotate since co-infusion of amino acids leads to a significant reduction (47%) in the mean radiation dose to the kidneys. The amino acid solution and ¹⁷⁷Lu-DOTA⁰-Tyr³-Octreotate are administered in parallel by peripheral vein infusion

TABLE 1
¹⁷⁷Lu-DOTA⁰-Tyr³-Octreotate Administration Schedule.

| | | Infusion | |
|---|---|---|---|
| | Starting | Rate | Duration |
| Preparation | time (h) | (ml/h) | (h) |
| Granisetron 3 mg (or alternative) | 0 | Bolus | — |
| Amino acids: 1L solution | 0 | 250* | 4 |
| ¹⁷⁷Lu-DOTA⁰-Tyr³-Octreotate | 0.5 | 50 | 0.5 |
| Saline solution 25 mL-(two | 1 | 50 | 0.5 |
| pump method)¹ | | | |

¹When the two pump method is used, ¹⁷⁷Lu-DOTA⁰-Tyr³-Octreotate is pumped directly into the infusion line. The infusion line must be flushed with at least 25 ml of sodium chloride 9 mg/ml (0.9%) solution for injection after the infusion of ¹⁷⁷Lu-DOTA⁰-Tyr³-Octreotate.
*The infusion rate of 250 ml/hr is suggested, but may be reduced at the discretion of the investigator.

### I-O Therapeutic Agent

The antibody will be administered once every 2 weeks until disease progression, patient withdrawal or toxicities. The antibody is administered intravenously and is administered first in combination studies. Wait 30 minutes before the next compound is administered (regardless of route of administration). Flush the intravenous line at end of infusion with appropriate amount of diluent (15-20 ml) to ensure that the total dose is administered. Administration of antibody may be given one day earlier or delayed up to 1 week due to holidays, inclement weather, conflicts, or similar reasons. The timing of subsequent administrations is then adjusted to maintain a 14 days-interval. Dose selection for the antibody should be assigned per patient or subject as outlined in the clinical protocol study drug dosing section.

### General Recommendations for Evaluation of Toxicities and Dose Delays/Modifications

Any patient who receives treatment on this protocol will be evaluable for toxicity. Toxicity will be assessed according to the NCI Common Toxicity Criteria for Adverse Events (CTCAE), version 4.03. Dose delays or dose modifications should be made according to the system showing the greatest degree of toxicity. Once the patient has a dose reduction due to toxicity, the dose will not be re-escalated. Dose delays and dose modifications will be made using the following recommendations.

At the discretion of the investigator, the study drugs may be held or dose modified independently if the observed AE is attributed to only one of the study drugs, while the patient continued to receive the drug not associated with the observed AE.

Dose modifications for ¹⁷⁷Lu-DOTA⁰-Tyr³-Octreotate are permitted according to the table below.

| Dose Level | ¹⁷⁷Lu-DOTA⁰-Tyr³-Octreotate |
|---|---|
| (DL) | (IV) |
| Level-1 | 3.7 GBq (100 mCi) every 8 |
| | weeks |
| Level 0 | 7.4 GBq (200 mCi) every 8 |
| | weeks |

No dose modifications are allowed for the antibody.

### Example 2: Clinical Phase I/II Trial of Combination of Lutathera and an Antibody as I-O Therapeutic Agents

### Primary Objectives

The primary objective of the phase I portion of the study is to determine the RP2D of ¹⁷⁷Lu-DOTA⁰-Tyr³-Octreotate when given in combination with anti-PD-1 checkpoint inhibitor antibody in patients with small-cell lung cancer or advanced or inoperable grade I-II pulmonary NETs.

The primary objective of the phase II portion of the study is to compare the PFS in patients with ES-SCLC who were not progressing to first-line treatment with platinum-based therapy, after receiving combination treatment of ¹⁷⁷Lu-DOTA⁰-Tyr³-Octreotate and antibody as a maintenance therapy versus observation.

### Secondary Objectives

To characterize the safety profile of ¹⁷⁷Lu-DOTA⁰-Tyr³-Octreotate in combination with antibody. [Applicable to both phase 1 and 2 portions]

In patients who were not progressing before initiating combo therapy: [Applicable to the phase 2 portion]
To assess DCR and ORR after treatment with ¹⁷⁷Lu-DOTA⁰-Tyr³-Octreotate plus antibody.
To assess OS
To assess whether the metabolic response seen on a NETSPOT® PET scan obtained on cycle 2 day 1 will predict response to study treatment.

### Inclusion Criteria (Phase I)

Patients must have cytologically or histologically confirmed relapsed or refractory extensive-disease small-cell lung cancer (ES-SCLC) or non-progressing ES-SCLC after first line chemotherapy, or advanced or inoperable grade I-II pulmonary NETs.

Patients with tumor tissue uptake during NETSPOT® PET that is equal to or higher than that in normal hepatic tissue (grade≥2) will be eligible. At the discretion of the principal investigator, patients with SCLC whose tumors have lower levels of uptake than liver during NETSPOT® PET may be eligible for the study.

Patients must have measurable disease by RECIST criteria, defined as at least one lesion that can be accurately measured in at least one dimension (longest diameter to be recorded) as >20 mm with conventional techniques or as >10 mm with spiral CT scan. See Section 7.1.2 for the evaluation of measurable disease.

Toxicities of prior therapy must be resolved to grade 1 or less as per Common Terminology Criteria for Adverse Events (CTCAE) version 4.03 with the exception of alopecia and grade 2, prior platinum-therapy related neuropathy.

Prior radiotherapy or radiosurgery (including prophylactic cranial radiation and/or thoracic radiation) must have been completed at least 2 weeks prior to randomization.

ECOG performance status of 0-1.

Adequate organ and bone marrow function (hemoglobin>9 g/dL; absolute neutrophil count>1.5×10⁹/L; platelet counts>100×10⁹/L; serum bilirubin<2×ULN; alanine aminotransferase (ALT) and aspartate aminotransferase (AST)<2.5×ULN or <5×ULN if liver metastases; calculated creatinine clearance>50 mL/min).

Life expectancy of at least 3 months.

Age>18 years.

### Inclusion Criteria (Phase II)

Patients must have cytologically or histologically confirmed ES-SCLC and must not have progressed after first line platinum-based chemotherapy regimen before randomization.

Patients with tumor tissue uptake during NETSPOT® PET that is equal to or higher than that in normal hepatic tissue (grade>2) will be eligible. It is recommended that NETSPOT® PET be obtained before initiation of chemotherapy, but NETSPOT® PET obtained during or after completion of chemotherapy could be used for screening purpose.

Patients must have measurable disease by RECIST criteria, defined as at least one lesion that can be accurately measured in at least one dimension (longest diameter to be recorded) as >20 mm with conventional techniques or as >10 mm with spiral CT scan. See Section 7.1.2 for the evaluation of measurable disease.

Toxicities of prior therapy must be resolved to grade 1 or less as per Common Terminology Criteria for Adverse Events (CTCAE) version 4.03 with the exception of alopecia and grade 2, prior platinum-therapy related neuropathy.

Prior radiotherapy or radiosurgery (including prophylactic cranial radiation and/or thoracic radiation) must have been completed at least 2 weeks prior to randomization.

For patients who do not receive radiotherapy after chemotherapy, the randomization must occur within 6 weeks of the last chemotherapy cycle. The study treatment must start within 2 weeks from randomization. For patients who receive radiotherapy (including prophylactic cranial radiation and/or thoracic radiation) after chemotherapy, the randomization must occur within 9 weeks of the last chemotherapy cycle but at least 2 weeks after completion of radiotherapy and the first dose of ⁷⁷Lu-DOTA⁰-Tyr³-Octreotate cannot be given within 8 weeks of radiotherapy.

ECOG performance status of 0-1.

Adequate organ and bone marrow function (hemoglobin>9 g/dL; absolute neutrophil count>1.5×10⁹/L; platelet counts>100×10⁹/L; serum bilirubin<2×ULN; alanine aminotransferase (ALT) and aspartate aminotransferase (AST)<2.5×ULN or <5×ULN if liver metastases; calculated creatinine clearance>50 mL/min).

Life expectancy of at least 3 months.

Age>18 years.

### Treatment Plan

### Treatment Dosage and Administration

### Phase I

### Dose Limiting Toxicity (DLT)

A DLT is defined as any toxicity not attributable to the disease or disease-related processes under investigation, which occurs from the first dose of study treatment (Day 1, Cycle 1) up to the last day of the cycle (Day 57). To be considered as DLT, it must be related to the study drugs (attributions: possible, probable, and definite) while fulfilling one of the following criteria as per the NCI Common Toxicity Criteria for Adverse Events (CTCAE) version 4.03:
Toxicity grade 2 for platelets and any other grade 3 or 4 toxicity, excluding
   Grade 3 diarrhea, nausea, or vomiting if it can be controlled with supportive therapy
   Grade 3 endocrinopathy that is managed with or without systemic corticosteroid therapy and/or hormone replacement therapy and the patient is asymptomatic.
Persistent (>21 days) non-hematologic grade 2 adverse events despite optimal medical management and treatment delay>21 days
Any other toxicity:
   if worse than baseline value, documented, clinically relevant and/or unacceptable, and is judged to be a DLT by the investigators
   if results in a protocol defined stopping criteria
   if results in disruption of dosing schedule

Patients experiencing DLT will be monitored weekly until toxicity stabilization, and then every two weeks until normalization.

### Dose Escalation and Treatment Duration

Treatment will be administered on an outpatient basis. A standard dose-escalation phase I design will be used. Three subjects will be enrolled at each dose level in the absence of DLT. Please find the details in the dose escalation table below.

### Dose Escalation Table

| Number of | |
|---|---|
| Patients with | |
| DLT at a | |
| Given Dose | |
| Level | Escalation Decision Rule |
| 0 out of 3 | Enter 3 patients at the next dose level. |
| ≥2 | Dose escalation will be stopped. This dose level will |
| | be declared the maximally administered dose (highest |
| | dose administered). Three additional patients will be |
| | entered at the next lowest dose level if only 3 patients |
| | were treated previously at that dose. |
| 1 out of 3 | Enter at least 3 more patients at this dose level. |
| | If 0 of these 3 patients experience DLT, proceed to |
| | the next dose level. |
| | If 1 or more of this group suffer DLT, then dose |
| | escalation is stopped, and this dose is declared |
| | the maximally administered dose. |
| | Three additional patients will be entered at |
| | the next lowest dose level if only 3 patients were |
| | treated previously at that dose. |
| ≤2 out of 6 at | This is generally the recommended phase 2 dose. |
| highest DL | At least 6 patients must be treated at the |
| | recommended phase 2 dose. |

Selection of the starting dose of ¹⁷⁷Lu-DOTA⁰-Tyr³-Octreotate and antibody is based on the results from previous clinical studies with each compound used as single agent and the fact that the combination has not been tested in clinical trials. The first dose of ¹⁷⁷Lu-DOTA⁰-Tyr³-Octreotate will be given two weeks after the first administration of antibody. Studies have shown that intravenous administration of amino acids has a renal protective effect [46]. An infusion of amino acids (lysine 2.5% and arginine 2.5% in 1 L 0.9% NaCl; 250 mL/h) will be started 30 minutes before the administration of ¹⁷⁷Lu-DOTA⁰-Tyr³-Octreotate and last 4 hours.

Antibody will be administered as a fixed dose of 240 mg as an intravenous infusion over 30 minutes every 2 weeks. Antibody will be given until progressive disease, patient withdrawal or toxicities.

### Dose-Finding

The following dose levels of ¹⁷⁷Lu-DOTA⁰-Tyr³-Octreotate could be explored in combination with antibody (Table 2):
Dose Level −1 (starting dose): 3.7 GBq (100 mCi)
Dose Level 0: 7.4 GBq (200 mCi)
¹⁷⁷Lu-DOTA⁰-Tyr³-Octreotate will be given every 8 weeks for a total 4 doses.

TABLE 2
Dose-escalation schedule.
Dose Escalation Schedule

| Dose Level | ¹⁷⁷Lu-DOTA⁰-Tyr³- | |
|---|---|---|
| (DL) | Octreotate (IV) | Antibody (IV) |
| Level-1 | 3.7 GBq (100 mCi) every | 240 mg every |
| (starting dose) | 8 weeks for 4 doses | 2 weeks |
| Level 0 | 7.4 GBq (200 mCi) every | 240 mg every |
| | 8 weeks for 4 doses | 2 weeks |

### Patient Replacement

Three patients within a dose level must be observed for one cycle (56 days) before accrual to the next higher dose level may begin. If a patient is withdrawn from the study prior to completing 56 days of therapy without experiencing a DLT prior to withdrawal, an additional patient may be added to that dose level.

### Phase II

The phase II portion will consist of patients with ES-SCLC that completed platinum based standard first-line chemotherapy (e.g. 4-6 cycles of platinum plus etoposide or irinotecan) without disease progression (responders plus stable disease) at the time of initiation of the combination therapy with ¹⁷⁷Lu-DOTA⁰-Tyr³-Octreotate and antibody. Eligible patients will then be randomly allocated in two arms: one will be treated with the combination of ¹⁷⁷Lu-DOTA⁰-Tyr³-Octreotate and antibody, and the other arm will continue be followed (observation) after completion the standard chemotherapy treatment. For patients who do not receive radiotherapy after chemotherapy, the randomization must occur within 6 weeks of the last chemotherapy cycle. The study treatment must start within 2 weeks from randomization. For patients who receive radiotherapy (including prophylactic cranial radiation and/or thoracic radiation) after chemotherapy, the randomization must occur within 9 weeks of the last chemotherapy cycle but at least 2 weeks after completion of radiotherapy and the first dose of ¹⁷⁷Lu-DOTA⁰-Tyr³-Octreotate cannot be given within 8 weeks of radiotherapy.
For all patients who have signed the informed consent form (ICF), a screening number will be assigned in chronological order starting with the lowest number available on site.
Patients will be identified by a unique patient identification number (Patient ID No.) composed of the center number (four digits) and the screening number (three digits).
The e-CRF will assign a unique randomization number to the patient, which will be used to link the patient to a treatment arm.
Randomization will be stratified according to NETSPOT® PET tumor uptake score (Grade 2, 3 and 4).

Courses are defined as 56 days of dosing. Antibody will be given until progressive disease, patient withdrawal, or toxicities. For patient randomized to the observation group, cross-over at the time of disease progression will be allowed, since the primary endpoint is PFS and not OS.

### Study Procedures

### Screening/Baseline Procedures

Subjects who meet all eligibility criteria will be enrolled in the study. Assessments performed exclusively to determine eligibility for this study will be done after obtaining informed consent. Assessments performed for clinical indications (not exclusively to determine study eligibility) may be used for baseline values even if the studies were done before informed consent was obtained. All screening procedures must be performed within 4 weeks prior to starting study drugs, unless otherwise stated. The screening procedures include:
Complete history and physical examination including vital signs, height, weight and ECOG performance score.
Baseline imaging studies: Patients should have a baseline radiographical evaluation with computed tomography (CT) scan of the chest/abdomen/pelvis, MRI or CT of the brain, and FDG-PET (skull base to mid-thigh). Two NETSPOT® PET scans will be performed, the first one within 4 weeks before the start of chemotherapy (preferable) or as soon as after initiation of chemotherapy. This scan will be used to evaluate SSTR2 expression and the patient's eligibility for the study. The second NETSPOT® PET scan will be conducted as far as possible from the end of chemotherapy (ideally within 1 week before the start of study treatment). This scan will be used for exploratory analysis on eventual SSTR2 expression modification with chemotherapy. If a patient presents after completion of chemotherapy and did not have a NETSPOT® PET scan performed before or during chemotherapy, a NETSPOT® PET scan will be obtained to determine the patient's eligibility. Outside imaging studies will be accepted at the discretion of the PI.
Electrocardiogram (EKG)
Laboratory evaluation (baseline tests to be obtained within one week prior to starting treatment unless otherwise noted)
   Hematological Profile: Complete blood count (CBC) with differential and platelet count, prothrombin time/international normalized ratio (PT/INR), activated partial thromboplastin time (aPTT).
   Biochemical Profile: Sodium, potassium, calcium, phosphorous, magnesium, blood urea nitrogen (BUN), creatinine, glucose, aspartate aminotransferase (AST), alanine aminotransferase (ALT), alkaline phosphatase, lactic acid dehydrogenase (LDH), bilirubin, albumin.
   Baseline glomerular filtration rate (GFR) calculation.
   Serum or urine beta-hCG for female patients of childbearing age within 24 hours prior to the start of study drug.
   Viral Markers: HBsAg, anti-HCV, anti-HIV within 3 months prior to starting treatment.
   Amiylase, lipase, thyroid finction test (TSH, free T3, free T4).

### Procedures During Treatment

Patients receiving study treatment will be followed every 2 weeks and the following will be done (unless otherwise indicated).
History and physical exam.
Laboratory evaluation: Hematologic profile (CBC with differential). Biochemical profile.
Thyroid function testing will be done approximately every 4 weeks for subjects receiving antibody.
Tumor imaging will be performed every 8 weeks (within a week of starting the next cycle).
NETSPOT® PET scan on cycle 2 day 1 (±3 days) to assess the metabolic response.
Serum or urine beta-hCG for female patients of childbearing age within 24 hours prior to the administration of ¹⁷⁷Lu-DOTA⁰-Tyr³-Octreotate.

Patients randomized to observation will be followed every 4 weeks and the following will be done.
History and physical exam.
Laboratory evaluation: Hematologic and biochemical profile.
Tumor imaging will be performed every 8 weeks.

After 30 days from treatment termination, the following will be obtained if the patient is available:
History and physical exam.
Laboratory evaluation: Hematologic and biochemical profile. Thyroid function testing.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. (canceled)

2. A method of treating a somatostatin receptor over-expressing cancer in a subject, comprising administering to the subject a combination of a peptide receptor radionuclide therapeutic (PRRT) agent and one or two immuno-oncology (I-O) therapeutic agent(s), wherein said I-O therapeutic agent(s) is(are) selected from the group consisting of an LAG-3 inhibitor, a TIM-3 inhibitor, a GITR angonists, a TGF-β inhibitor, an IL15/IL-15RA complex, and a PD-1 inhibitor, wherein said PD-1 inhibitor is selected from the group consisting of Spartalizumab, Pembrolizumab, Pidilizumab, Durvalomab, Atezolizumab, Avelumab, MED10680, REGN2810, TSR-042, PF-06801591, BGB-A317, BGB-108, INCSHR1210, and AMP-224.

3. The method of claim 2, wherein the PRRT agent comprises the radionuclide Lutetium-177 (177Lu) and a somatostatin receptor binding molecule linked to a chelating agent.

4. The method of claim 3, wherein the somatostatin receptor binding molecule is selected from the group consisting of octreotide, octreotate, lanreotide, vapreotide, pasireotide, and satoreotide.

5. The method of claim 4, wherein the chelating agent is 1,4,7,10-Tetraazacyclododecane-1,4,7,10-tetraacetic acid (DOTA).

6. The method of claim 3, wherein the somatostatin receptor binding molecule linked to the chelating agent is selected from the group consisting of DOTA-OC: [DOTA0, D-Phe1]octreotide, DOTA-TOC: [DOTA0, D-Phe1, Tyr3]octreotide (i.e. edotreotide), DOTA-NOC: [DOTA0, D-Phe1,1-NaI3]octreotide, DOTA-TATE: [DOTA0, D-Phe1, Tyr3]octreotate (i.e. oxodotreotide), DOTA-LAN: [DOTA0, D-μ-NaI1]lanreotide, DOTA-VAP: [DOTA0, D-Phe1, Tyr3]vapreotide, satoreotide trizoxetan, and satoreotide tetraxetan.

7. The method of claim 2, wherein the PRRT agent is lutetium (177Lu) oxodotreotide (i.e. 177Lu[DOTA0, D-Phe1, Tyr3]octreotate).

8. The method of claim 3, wherein the PRRT agent is formulated as a pharmaceutical aqueous solution comprising:
(a) a complex formed by
(ai) the radionuclide 177Lu (Lutetium-177) in a concentration that it provides a volumetric radioactivity of from 250 to 500 MBq/mL, and
(aii) the DOTA linked somatostatin receptor binding peptide;
(b) the stabilizers against radiolytic degradation (bi) gentisic acid in a concentration of from 0.5 to 1 mg/mL and (bii) ascorbic acid in a concentration of from 2.0 to 5.0 mg/mL;
(c) diethylentriaminepentaacetic acid (DTPA) or a salt thereof in a concentration of from 0.01 to 0.10 mg/mL; and
(d) an acetate buffer composed of:
(di) acetic acid in a concentration of from 0.3 to 0.7 mg/mL; and
(dii) sodium acetate in a concentration from 0.4 to 0.9 mg/mL;
preferably said acetate buffer provides for a pH of from 4.5 to 6.0, preferably from 5.0 to 5.5.

9. The method of claim 8, wherein gentisic acid is present during the complex formation of components (ai) and (aii) and ascorbic acid added after the complex formation of components (ai) and (aii).

10. The method of claim 2, wherein the LAG-3 inhibitor is chosen from LAG525, BMS-986016, or TSR-033.

11. The method of claim 2, wherein the TIM-3 inhibitor is MBG453 or TSR-022.

12. The method of claim 2, wherein the GITR agonist is chosen from GWN323, BMS-986156, MK-4166, MK-1248, TRX518, INCAGN1876, AMG 228, or INBRX-110.

13. The method of claim 2, wherein the TGF-β inhibitor is XOMA 089 or fresolimumab.

14. The method of claim 2, wherein the IL-15/IL-15RA complex is chosen from NIZ985, ATL-803 or CYP0150.

15. The method of claim 2, comprising one or two further anti-cancer agent(s).

16. The method of claim 15, wherein the further anti-cancer agent(s) is (are) selected from the group consisting of octreotide, lanreotide, vaproreotide, pasireotide, satoreotide, everolimus, temozolomide, telotristat, sunitinib, sulfatinib, ribociclib, entinostat, and pazopanib.

17. The method of claim 2, wherein the somatostatin receptor over-expressing cancer is a neuroendocrine tumor (NET).

18. The method of claim 17, wherein the neuroendocrine tumor (NET) is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, Non-Hodgkin lymphoma, Hodgkin lymphoma, Head & Neck tumor, urothelial carcinoma (bladder), Renal Cell Carcinoma, Hepatocellular Carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, and esthesioneuroblastoma.

19. The method of claim 17, wherein the neuroendocrine tumor (NET) is selected from the group consisting of functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

20. The method of claim 7, wherein the I-O therapeutric agent is Atezolizumab.

21. A formulation comprising a peptide receptor radionuclide therapeutic (PRRT) agent, and one or two immuno-oncology (I-O) therapeutic agent(s), wherein said I-O therapeutic agent(s) is(are) selected from the group consisting of an LAG-3 inhibitor, a TIM-3 inhibitor, a GITR angonists, a TGF-β inhibitor, an IL15/IL-15RA complex, and a PD-1 inhibitor, wherein said PD-1 inhibitor is selected from the group consisting of Spartalizumab, Pembrolizumab, Pidilizumab, Durvalomab, Atezolizumab.
